SEQUENCE LISTING (275 sequences)

SEQ ID NO: 1 - protein, 21 aa, synthetic construct
  REGION 1..21: note = Description of Artificial Sequence: Synthetic peptide
MALPVTALLLPLALLLHAARP

SEQ ID NO: 2 - protein, 126 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 3 - protein, 166 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 4 - protein, 118 aa, synthetic construct
  REGION 1..118: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 5 - protein, 110 aa, synthetic construct
  REGION 1..110: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSELLLVIFQ

SEQ ID NO: 6 - protein, 112 aa, synthetic construct
  REGION 1..112: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEPDLLLVIFQ

SEQ ID NO: 7 - protein, 113 aa, synthetic construct
  REGION 1..113: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEPDLLLVIFQ

SEQ ID NO: 8 - protein, 114 aa, synthetic construct
  REGION 1..114: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYPDLLLVIFQ

SEQ ID NO: 9 - protein, 115 aa, synthetic construct
  REGION 1..115: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNPDLLLVIFQ

SEQ ID NO: 10 - protein, 118 aa, synthetic construct
  REGION 1..118: note = Description of Artificial Sequence: Synthetic polypeptide
QLCKFCDNRTSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 11 - protein, 118 aa, synthetic construct
  REGION 1..118: note = Description of Artificial Sequence: Synthetic polypeptide
QLCKFCDVRFSTCDNQTSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 12 - protein, 118 aa, synthetic construct
  REGION 1..118: note = Description of Artificial Sequence: Synthetic polypeptide
QLCKFCDVRFSTCDNQKSCMSNCSITSICENPTEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 13 - protein, 118 aa, synthetic construct
  REGION 1..118: note = Description of Artificial Sequence: Synthetic polypeptide
QLCKFCDVRFSTCDNQKSCMSNCSITSICEKPNETCVAVWRKNDENITLETVCHDPKLPYHDFILEDAASPKCIMKEKKKPGETFFMCSCSSDECNDNIIFSEEYNTSNPDLLLVIFQ

SEQ ID NO: 14 - protein, 118 aa, synthetic construct
  REGION 1..118: note = Description of Artificial Sequence: Synthetic polypeptide
QLCKFCDKRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 15 - protein, 118 aa, synthetic construct
  REGION 1..118: note = Description of Artificial Sequence: Synthetic polypeptide
QLCKFCDVRFSTCDNQKSCMSNCSITSICEKPQEVCVAVWRKNDKNITLETVCHDPKLPYHDFILEDAASPKCIMKEKKKPGETFFMCSCSSDECNDNIIFSEEYNTSNPDLLLVIFQ

SEQ ID NO: 16 - protein, 101 aa, synthetic construct
  REGION 1..101: note = Description of Artificial Sequence: Synthetic polypeptide
QLCKFCDVRFSTCDNQKSCMSNCSITSICEKPQEVCVAVWRKNDENITLETVCHDPKLPYHNFTLEDAASPKCIMKEKKKPGETFFMCSCSSDECNDNIIF

SEQ ID NO: 17 - protein, 101 aa, synthetic construct
  REGION 1..101: note = Description of Artificial Sequence: Synthetic polypeptide
QLCKFCDVRFSTCDNQKSCMSNCSITSICEKPQEVCVAVWRKNDENITLETVCHDPKLPYHDFNLTDAASPKCIMKEKKKPGETFFMCSCSSDECNDNIIF

SEQ ID NO: 18 - protein, 101 aa, synthetic construct
  REGION 1..101: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKNKTKPGETFFMCSCSSDECNDNIIF

SEQ ID NO: 19 - protein, 101 aa, synthetic construct
  REGION 1..101: note = Description of Artificial Sequence: Synthetic polypeptide
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNITF

SEQ ID NO: 20 - protein, 101 aa, synthetic construct
  REGION 1..101: note = Description of Artificial Sequence: Synthetic polypeptide
QLCKFCDVRFSTCDNQKSCMSNCSITSICEKPQEVCVAVWRKNDENITLETVCHDPKLPYHDFELEDAASPKCIMKEKKKPGETFFMCSCSSDECNDNIIF

SEQ ID NO: 21 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 22 - protein, 918 aa, Homo sapiens [sequence omitted]

SEQ ID NO: 23 - protein, 622 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 24 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 25 - protein, 863 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 26 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 27 - protein, 97 aa, synthetic construct
  REGION 1..97: note = Description of Artificial Sequence: Synthetic polypeptide
LTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWE

SEQ ID NO: 28 - protein, 92 aa, synthetic construct
  REGION 1..92: note = Description of Artificial Sequence: Synthetic polypeptide
TTPKFAQGEIEAIVVPVCLAFLLTTLLGVLFCFNKRDLIKKHIWPNVPDPSKSHIAQWSPHTPPRHNFNSKDQMYSDGNFTDVSVVEIEAND

SEQ ID NO: 29 - protein, 105 aa, synthetic construct
  REGION 1..105: note = Description of Artificial Sequence: Synthetic polypeptide
SDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 30 - protein, 104 aa, synthetic construct
  REGION 1..104: note = Description of Artificial Sequence: Synthetic polypeptide
SDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 31 - protein, 103 aa, synthetic construct
  REGION 1..103: note = Description of Artificial Sequence: Synthetic polypeptide
SDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 32 - protein, 104 aa, synthetic construct
  REGION 1..104: note = Description of Artificial Sequence: Synthetic polypeptide
SDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 33 - protein, 102 aa, synthetic construct
  REGION 1..102: note = Description of Artificial Sequence: Synthetic polypeptide
SDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 34 - protein, 101 aa, synthetic construct
  REGION 1..101: note = Description of Artificial Sequence: Synthetic polypeptide
SDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 35 - protein, 102 aa, synthetic construct
  REGION 1..102: note = Description of Artificial Sequence: Synthetic polypeptide
SDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 36 - protein, 100 aa, synthetic construct
  REGION 1..100: note = Description of Artificial Sequence: Synthetic polypeptide
SDPTRVETATETVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 37 - protein, 99 aa, synthetic construct
  REGION 1..99: note = Description of Artificial Sequence: Synthetic polypeptide
SDPTRVETATETTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 38 - protein, 98 aa, synthetic construct
  REGION 1..98: note = Description of Artificial Sequence: Synthetic polypeptide
SDPTRVETATETALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 39 - protein, 97 aa, synthetic construct
  REGION 1..97: note = Description of Artificial Sequence: Synthetic polypeptide
SDPTRVETATETLHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 40 - protein, 96 aa, synthetic construct
  REGION 1..96: note = Description of Artificial Sequence: Synthetic polypeptide
SDPTRVETATETHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 41 - protein, 95 aa, synthetic construct
  REGION 1..95: note = Description of Artificial Sequence: Synthetic polypeptide
SDPTRVETATETLVLGLSAVLGLLLLRWQFPAHYRRLRHALWPSLPDLHRVLGQYLRDTAALSPPKATVSDTCEEVEPSLLEILPKSSERTPLPL

SEQ ID NO: 42 - protein, 94 aa, synthetic construct
  REGION 1..94: note = Description of Artificial Sequence: Synthetic polypeptide
SDPTRVETATETVLGLSAVLGLLLLRWQFPAHYRRLRHALWPSLPDLHRVLGQYLRDTAALSPPKATVSDTCEEVEPSLLEILPKSSERTPLPL

SEQ ID NO: 43 - protein, 93 aa, synthetic construct
  REGION 1..93: note = Description of Artificial Sequence: Synthetic polypeptide
SDPTRVETATETLGLSAVLGLLLLRWQFPAHYRRLRHALWPSLPDLHRVLGQYLRDTAALSPPKATVSDTCEEVEPSLLEILPKSSERTPLPL

SEQ ID NO: 44 - protein, 92 aa, synthetic construct
  REGION 1..92: note = Description of Artificial Sequence: Synthetic polypeptide
SDPTRVETATETGLSAVLGLLLLRWQFPAHYRRLRHALWPSLPDLHRVLGQYLRDTAALSPPKATVSDTCEEVEPSLLEILPKSSERTPLPL

SEQ ID NO: 45 - protein, 91 aa, synthetic construct
  REGION 1..91: note = Description of Artificial Sequence: Synthetic polypeptide
SDPTRVETATETLSAVLGLLLLRWQFPAHYRRLRHALWPSLPDLHRVLGQYLRDTAALSPPKATVSDTCEEVEPSLLEILPKSSERTPLPL

SEQ ID NO: 46 - protein, 90 aa, synthetic construct
  REGION 1..90: note = Description of Artificial Sequence: Synthetic polypeptide
SDPTRVETATETSAVLGLLLLRWQFPAHYRRLRHALWPSLPDLHRVLGQYLRDTAALSPPKATVSDTCEEVEPSLLEILPKSSERTPLPL

SEQ ID NO: 47 - protein, 89 aa, synthetic construct
  REGION 1..89: note = Description of Artificial Sequence: Synthetic polypeptide
SDPTRVETATETAVLGLLLLRWQFPAHYRRLRHALWPSLPDLHRVLGQYLRDTAALSPPKATVSDTCEEVEPSLLEILPKSSERTPLPL

SEQ ID NO: 48 - protein, 88 aa, synthetic construct
  REGION 1..88: note = Description of Artificial Sequence: Synthetic polypeptide
SDPTRVETATETVLGLLLLRWQFPAHYRRLRHALWPSLPDLHRVLGQYLRDTAALSPPKATVSDTCEEVEPSLLEILPKSSERTPLPL

SEQ ID NO: 49 - protein, 87 aa, synthetic construct
  REGION 1..87: note = Description of Artificial Sequence: Synthetic polypeptide
SDPTRVETATETLGLLLLRWQFPAHYRRLRHALWPSLPDLHRVLGQYLRDTAALSPPKATVSDTCEEVEPSLLEILPKSSERTPLPL

SEQ ID NO: 50 - protein, 106 aa, synthetic construct
  REGION 1..106: note = Description of Artificial Sequence: Synthetic polypeptide
SDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 51 - protein, 107 aa, synthetic construct
  REGION 1..107: note = Description of Artificial Sequence: Synthetic polypeptide
SDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 52 - protein, 108 aa, synthetic construct
  REGION 1..108: note = Description of Artificial Sequence: Synthetic polypeptide
SDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 53 - protein, 109 aa, synthetic construct
  REGION 1..109: note = Description of Artificial Sequence: Synthetic polypeptide
SDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 54 - protein, 110 aa, synthetic construct
  REGION 1..110: note = Description of Artificial Sequence: Synthetic polypeptide
SDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 55 - protein, 111 aa, synthetic construct
  REGION 1..111: note = Description of Artificial Sequence: Synthetic polypeptide
SDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 56 - protein, 112 aa, synthetic construct
  REGION 1..112: note = Description of Artificial Sequence: Synthetic polypeptide
SDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 57 - protein, 113 aa, synthetic construct
  REGION 1..113: note = Description of Artificial Sequence: Synthetic polypeptide
SDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 58 - protein, 105 aa, synthetic construct
  REGION 1..105: note = Description of Artificial Sequence: Synthetic polypeptide
SDPTRVETATETAWISLVTALHLVLGLSAVLGLLLLRKQFPAHYRRLRHALWPSLPDLHRVLGQYLRDTAALSPPKATVSDTCEEVEPSLLEILPKSSERTPLPL

SEQ ID NO: 59 - protein, 105 aa, synthetic construct
  REGION 1..105: note = Description of Artificial Sequence: Synthetic polypeptide
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPL

SEQ ID NO: 60 - protein, 105 aa, synthetic construct
  REGION 1..105: note = Description of Artificial Sequence: Synthetic polypeptide
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 61 - protein, 95 aa, synthetic construct
  REGION 1..95: note = Description of Artificial Sequence: Synthetic polypeptide
SDPTRVETATEHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 62 - protein, 94 aa, synthetic construct
  REGION 1..94: note = Description of Artificial Sequence: Synthetic polypeptide
SDPTRVETATHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 63 - protein, 93 aa, synthetic construct
  REGION 1..93: note = Description of Artificial Sequence: Synthetic polypeptide
SDPTRVETAHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 64 - protein, 92 aa, synthetic construct
  REGION 1..92: note = Description of Artificial Sequence: Synthetic polypeptide
SDPTRVETHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 65 - protein, 91 aa, synthetic construct
  REGION 1..91: note = Description of Artificial Sequence: Synthetic polypeptide
SDPTRVEHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 66 - protein, 90 aa, synthetic construct
  REGION 1..90: note = Description of Artificial Sequence: Synthetic polypeptide
SDPTRVHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 67 - protein, 89 aa, synthetic construct
  REGION 1..89: note = Description of Artificial Sequence: Synthetic polypeptide
SDPTRHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 68 - protein, 88 aa, synthetic construct
  REGION 1..88: note = Description of Artificial Sequence: Synthetic polypeptide
SDPTHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 69 - protein, 87 aa, synthetic construct
  REGION 1..87: note = Description of Artificial Sequence: Synthetic polypeptide
SDPHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 70 - protein, 86 aa, synthetic construct
  REGION 1..86: note = Description of Artificial Sequence: Synthetic polypeptide
SDHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 71 - protein, 85 aa, synthetic construct
  REGION 1..85: note = Description of Artificial Sequence: Synthetic polypeptide
SHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 72 - protein, 84 aa, synthetic construct
  REGION 1..84: note = Description of Artificial Sequence: Synthetic polypeptide
HLVLGLSAVLGLLLLRWQFPAHYRRLRHALWPSLPDLHRVLGQYLRDTAALSPPKATVSDTCEEVEPSLLEILPKSSERTPLPL

SEQ ID NO: 73 - protein, 102 aa, synthetic construct
  REGION 1..102: note = Description of Artificial Sequence: Synthetic polypeptide
SDPTRVETATETAWISLVHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 74 - protein, 100 aa, synthetic construct
  REGION 1..100: note = Description of Artificial Sequence: Synthetic polypeptide
SDPTRVETATETAWISHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 75 - protein, 101 aa, synthetic construct
  REGION 1..101: note = Description of Artificial Sequence: Synthetic polypeptide
SDPTRVETATETAWISPHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 76 - protein, 102 aa, synthetic construct
  REGION 1..102: note = Description of Artificial Sequence: Synthetic polypeptide
SDPTRVETATETAWISPPHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 77 - protein, 99 aa, synthetic construct
  REGION 1..99: note = Description of Artificial Sequence: Synthetic polypeptide
SDPTRVETATETAWIHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 78 - protein, 100 aa, synthetic construct
  REGION 1..100: note = Description of Artificial Sequence: Synthetic polypeptide
SDPTRVETATETAWIPHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 79 - protein, 101 aa, synthetic construct
  REGION 1..101: note = Description of Artificial Sequence: Synthetic polypeptide
SDPTRVETATETAWIPPHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 80 - protein, 144 aa, synthetic construct
  REGION 1..144: note = Description of Artificial Sequence: Synthetic polypeptide
ARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 81 - protein, 219 aa, synthetic construct
  REGION 1..219: note = Description of Artificial Sequence: Synthetic polypeptide
VTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 82 - protein, 50 aa, synthetic construct
  REGION 1..50: note = Description of Artificial Sequence: Synthetic polypeptide
ISTIATVEETNQTDEDHKKYSSQTSQDSGNYSNEDESESKTSEELQQDFV

SEQ ID NO: 83 - protein, 206 aa, synthetic construct
  REGION 1..206: note = Description of Artificial Sequence: Synthetic polypeptide
KKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 84 - protein, 256 aa, synthetic construct
  REGION 1..256: note = Description of Artificial Sequence: Synthetic polypeptide
ISTIATVEETNQTDEDHKKYSSQTSQDSGNYSNEDESESKTSEELQQDFVKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 85 - protein, 221 aa, synthetic construct
  REGION 1..221: note = Description of Artificial Sequence: Synthetic polypeptide
RGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 86 - protein, 35 aa, synthetic construct
  REGION 1..35: note = Description of Artificial Sequence: Synthetic polypeptide
IPPKGGALGEGPGASPCNQHSPYWAPPCYTLKPET

SEQ ID NO: 87 - protein, 166 aa, synthetic construct
  REGION 1..166: note = Description of Artificial Sequence: Synthetic polypeptide
TALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 88 - protein, 217 aa, synthetic construct
  REGION 1..217: note = Description of Artificial Sequence: Synthetic polypeptide
PRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 89 - protein, 286 aa, synthetic construct
  REGION 1..286: note = Description of Artificial Sequence: Synthetic polypeptide
PDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 90 - protein, 170 aa, synthetic construct
  REGION 1..170: note = Description of Artificial Sequence: Synthetic polypeptide
WGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 91 - protein, 203 aa, synthetic construct
  REGION 1..203: note = Description of Artificial Sequence: Synthetic polypeptide
AVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 92 - protein, 144 aa, synthetic construct
  REGION 1..144: note = Description of Artificial Sequence: Synthetic polypeptide
ARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 93 - protein, 231 aa, synthetic construct
  REGION 1..231: note = Description of Artificial Sequence: Synthetic polypeptide
VIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 94 - protein, 218 aa, synthetic construct
  REGION 1..218: note = Description of Artificial Sequence: Synthetic polypeptide
VIQGDERMHLPSPTDSNFFRALMDEEDMDDVVDADEYLIPQQGFFSSPSTSRTPLLSSLSATSNNSTVACIDRNGLQSCPIKEDSFLQRIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 95 - protein, 54 aa, synthetic construct
  REGION 1..54: note = Description of Artificial Sequence: Synthetic polypeptide
VIQGDERMHLPSPTDSNFFRALMDEEDMDDVVDADEYLIPQQGFFSSPSTSRTP

SEQ ID NO: 96 - protein, 54 aa, synthetic construct
  REGION 1..54: note = Description of Artificial Sequence: Synthetic polypeptide
NNSTVACIDRNGLQSCPIKEDSFLQRIDDTFLPVPEYINQSVPKRPAGSVQNPV

SEQ ID NO: 97 - protein, 54 aa, synthetic construct
  REGION 1..54: note = Description of Artificial Sequence: Synthetic polypeptide
KEDSFLQRIDDTFLPVPEFINQSVPKRPAGSVQNPVYHNQPLNPAPSRDPHFQD

SEQ ID NO: 98 - protein, 54 aa, synthetic construct
  REGION 1..54: note = Description of Artificial Sequence: Synthetic polypeptide
VPEFINQSVPKRPAGSVQNPVFHNQPLNPAPSRDPHYQDPHSTAVGNPEYLNTV

SEQ ID NO: 99 - protein, 54 aa, synthetic construct
  REGION 1..54: note = Description of Artificial Sequence: Synthetic polypeptide
PEYLNTVQPTCVNSTFDSPAHWAQKGSHQISLDNPDYQQDFFPKEAKPNGIFKG

SEQ ID NO: 100 - protein, 54 aa, synthetic construct
  REGION 1..54: note = Description of Artificial Sequence: Synthetic polypeptide
WAQKGSHQISLDNPDFQQDFFPKEAKPNGIFKGSTAENAEYLRVAPQSSEFIGA

SEQ ID NO: 101 - protein, 51 aa, synthetic construct
  REGION 1..51: note = Description of Artificial Sequence: Synthetic polypeptide
SDPKPENPACPWTVLPAGDLPTHDGYLPSNIDDLPSHEAPLADSLEELEPQ

SEQ ID NO: 102 - protein, 41 aa, synthetic construct
  REGION 1..41: note = Description of Artificial Sequence: Synthetic polypeptide
ACDAPILSSSRSLDCRESGKNGPHVYQDLLLSLGTTNSTLP

SEQ ID NO: 103 - protein, 36 aa, synthetic construct
  REGION 1..36: note = Description of Artificial Sequence: Synthetic polypeptide
GILTLNPVAQGQPILTSLGSNQEEAYVTMSSFYQNQ

SEQ ID NO: 104 - protein, 68 aa, synthetic construct
  REGION 1..68: note = Description of Artificial Sequence: Synthetic polypeptide
ACDAPILSSSRSLDCRESGKNGPHVYQDLLLSLGTTNSTLPQGQPILTSLGSNQEEAYVTMSSFYQNQ

SEQ ID NO: 105 - protein, 36 aa, synthetic construct
  REGION 1..36: note = Description of Artificial Sequence: Synthetic polypeptide
GILTLNPVAQGQPILTSLGSNQEEAYVTMSSFFQNQ

SEQ ID NO: 106 - protein, 68 aa, synthetic construct
  REGION 1..68: note = Description of Artificial Sequence: Synthetic polypeptide
ACDAPILSSSRSLDCRESGKNGPHVYQDLLLSLGTTNSTLPQGQPILTSLGSNQEEAYVTMSSFFQNQ

SEQ ID NO: 107 - protein, 41 aa, synthetic construct
  REGION 1..41: note = Description of Artificial Sequence: Synthetic polypeptide
DEGVAGAPTGSSPQPLQPLSGEDDAYCTFPSRDDLLLFSPS

SEQ ID NO: 108 - protein, 34 aa, synthetic construct
  REGION 1..34: note = Description of Artificial Sequence: Synthetic polypeptide
GQGEFRALNARLPLNTDAYLSLQELQGQDPTHLV

SEQ ID NO: 109 - protein, 82 aa, synthetic construct
  REGION 1..82: note = Description of Artificial Sequence: Synthetic polypeptide
QQDKVPEPASLSSNHSLTSCFTNQGYFFFHLPDALEIEACQDEGVAGAPTGSSPQPLQPLSGEDDAYCTFPSRDDLLLFSPS

SEQ ID NO: 110 - protein, 75 aa, synthetic construct
  REGION 1..75: note = Description of Artificial Sequence: Synthetic polypeptide
QQDKVPEPASLSSNHSLTSCFTNQGYFFFHLPDALEIEACQGQGEFRALNARLPLNTDAYLSLQELQGQDPTHLV

SEQ ID NO: 111 - protein, 75 aa, synthetic construct
  REGION 1..75: note = Description of Artificial Sequence: Synthetic polypeptide
DEGVAGAPTGSSPQPLQPLSGEDDAYCTFPSRDDLLLFSPSGQGEFRALNARLPLNTDAYLSLQELQGQDPTHLV

SEQ ID NO: 112 - protein, 116 aa, synthetic construct
  REGION 1..116: note = Description of Artificial Sequence: Synthetic polypeptide
QQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 113 - protein, 43 aa, synthetic construct
  REGION 1..43: note = Description of Artificial Sequence: Synthetic polypeptide
KKKVWDYNYDDESDSDTEAAPRTSGGGYTMHGLTVRPLGQASA

SEQ ID NO: 114 - protein, 29 aa, synthetic construct
  REGION 1..29: note = Description of Artificial Sequence: Synthetic peptide
EGLWSEDAPSDQSDTSESDVDLGDGYIMR

SEQ ID NO: 115 - protein, 72 aa, synthetic construct
  REGION 1..72: note = Description of Artificial Sequence: Synthetic polypeptide
KKKVWDYNYDDESDSDTEAAPRTSGGGYTMHGLTVRPLGQASAEGLWSEDAPSDQSDTSESDVDLGDGYIMR

SEQ ID NO: 116 - protein, 156 aa, synthetic construct
  REGION 1..156: note = Description of Artificial Sequence: Synthetic polypeptide
ASESPADEEEQWSDDFDSDYENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 117 - protein, 156 aa, synthetic construct
  REGION 1..156: note = Description of Artificial Sequence: Synthetic polypeptide
ASESPADEEEQWSDDFDSDFENPDEHSDSEMYVMPAEENADDSYEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 118 - protein, 156 aa, synthetic construct
  REGION 1..156: note = Description of Artificial Sequence: Synthetic polypeptide
ASESPADEEEQWSDDFDSDFENPDEHSDSEMYVMPAEENADDSFEPPPVEQETRPVHPALPFARGEYIDNRSSQRHSPPFSKTLPSKPSWPSEKARLTSTLPALTALQKPQVPPKPKGLLEDEADYVVPVEDNDENYIHPTESSSPPPEKAPMVNR

SEQ ID NO: 119 - protein, 170 aa, synthetic construct; identical to SEQ ID NO: 90
  REGION 1..170: note = Description of Artificial Sequence: Synthetic polypeptide

SEQ ID NO: 120 - protein, 149 aa, synthetic construct
  REGION 1..149: note = Description of Artificial Sequence: Synthetic polypeptide
VTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 121 - protein, 40 aa, synthetic construct
  REGION 1..40: note = Description of Artificial Sequence: Synthetic polypeptide
WDKGERTEPLEKTELPEGAPELALDTELSLEDGDRCKAKM

SEQ ID NO: 122 - protein, 275 aa, synthetic construct
  REGION 1..275: note = Description of Artificial Sequence: Synthetic polypeptide
VSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 123 - protein, 487 aa, synthetic construct
  REGION 1..487: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 124 - protein, 486 aa, synthetic construct
  REGION 1..486: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 125 - protein, 485 aa, synthetic construct
  REGION 1..485: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 126 - protein, 486 aa, synthetic construct
  REGION 1..486: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 127 - protein, 484 aa, synthetic construct
  REGION 1..484: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 128 - protein, 483 aa, synthetic construct
  REGION 1..483: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 129 - protein, 484 aa, synthetic construct
  REGION 1..484: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 130 - protein, 482 aa, synthetic construct
  REGION 1..482: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 131 - protein, 481 aa, synthetic construct
  REGION 1..481: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 132 - protein, 480 aa, synthetic construct
  REGION 1..480: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 133 - protein, 479 aa, synthetic construct
  REGION 1..479: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 134 - protein, 478 aa, synthetic construct
  REGION 1..478: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 135 - protein, 477 aa, synthetic construct
  REGION 1..477: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 136 - protein, 476 aa, synthetic construct
  REGION 1..476: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 137 - protein, 475 aa, synthetic construct
  REGION 1..475: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 138 - protein, 474 aa, synthetic construct
  REGION 1..474: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 139 - protein, 473 aa, synthetic construct
  REGION 1..473: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 140 - protein, 472 aa, synthetic construct
  REGION 1..472: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 141 - protein, 471 aa, synthetic construct
  REGION 1..471: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 142 - protein, 470 aa, synthetic construct
  REGION 1..470: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 143 - protein, 469 aa, synthetic construct
  REGION 1..469: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 144 - protein, 488 aa, synthetic construct
  REGION 1..488: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 145 - protein, 489 aa, synthetic construct
  REGION 1..489: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 146 - protein, 490 aa, synthetic construct
  REGION 1..490: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 147 - protein, 491 aa, synthetic construct
  REGION 1..491: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 148 - protein, 492 aa, synthetic construct
  REGION 1..492: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 149 - protein, 493 aa, synthetic construct
  REGION 1..493: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 150 - protein, 494 aa, synthetic construct
  REGION 1..494: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 151 - protein, 495 aa, synthetic construct
  REGION 1..495: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSDPKPENPACPWTVLPAGDLPTHDGYLPSNIDDLPSHEAPLADSLEELEPQ

SEQ ID NO: 152 - protein, 13 aa, synthetic construct
  REGION 1..13: note = Description of Artificial Sequence: Synthetic peptide
KPIPNPLLGLDST

SEQ ID NO: 153 - protein, 246 aa, synthetic construct
  REGION 1..246: note = Description of Artificial Sequence: Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 154 - protein, 69 aa, synthetic construct
  REGION 1..69: note = Description of Artificial Sequence: Synthetic polypeptide
TTTPAPRPPTPAPTIASQPLSLRPEACRPAAGGAVHTRGLDFACDIYIWAPLAGTCGVLLLSLVITLYC

SEQ ID NO: 155 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 156 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 233 aa, synthetic construct
  REGION 1..233: note = Description of Artificial Sequence: Synthetic polypeptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 158 - protein, 22 aa, synthetic construct
  REGION 1..22: note = Description of Artificial Sequence: Synthetic peptide
GSGATNFSLLKQAGDVEENPGP

SEQ ID NO: 159 - protein, 143 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 160 - protein, 105 aa, synthetic construct
  REGION 1..105: note = Description of Artificial Sequence: Synthetic polypeptide
SDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 161 - protein, 197 aa, synthetic construct
  REGION 1..197: note = Description of Artificial Sequence: Synthetic polypeptide
ARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 162 - protein, 491 aa, synthetic construct
  REGION 1..491: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 163 - protein, 491 aa, synthetic construct
  REGION 1..491: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 164 - protein, 369 aa, synthetic construct
  REGION 1..369: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 165 - protein, 491 aa, synthetic construct
  REGION 1..491: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 166 - protein, 369 aa, synthetic construct
  REGION 1..369: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 167 - protein, 348 aa, synthetic construct
  REGION 1..348: note = Description of Artificial Sequence: Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 168 - protein, 348 aa, synthetic construct
  REGION 1..348: note = Description of Artificial Sequence: Synthetic polypeptide
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 169 - protein, 438 aa, synthetic construct
  REGION 1..438: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 170 - protein, 438 aa, synthetic construct
  REGION 1..438: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 171 - protein, 410 aa, synthetic construct
  REGION 1..410: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 172 - protein, 490 aa, synthetic construct
  REGION 1..490: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETWISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 173 - protein, 489 aa, synthetic construct
  REGION 1..489: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 174 - protein, 490 aa, synthetic construct
  REGION 1..490: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 175 - protein, 488 aa, synthetic construct
  REGION 1..488: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETSLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 176 - protein, 487 aa, synthetic construct
  REGION 1..487: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 177 - protein, 488 aa, synthetic construct
  REGION 1..488: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETILVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 178 - protein, 486 aa, synthetic construct
  REGION 1..486: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 179 - protein, 485 aa, synthetic construct
  REGION 1..485: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 180 - protein, 484 aa, synthetic construct
  REGION 1..484: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 181 - protein, 483 aa, synthetic construct
  REGION 1..483: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 182 - protein, 482 aa, synthetic construct
  REGION 1..482: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 183 - protein, 481 aa, synthetic construct
  REGION 1..481: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 184 - protein, 480 aa, synthetic construct
  REGION 1..480: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 185 - protein, 479 aa, synthetic construct
  REGION 1..479: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 186 - protein, 478 aa, synthetic construct
  REGION 1..478: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 187 - protein, 477 aa, synthetic construct
  REGION 1..477: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 188 - protein, 476 aa, synthetic construct
  REGION 1..476: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 189 - protein, 475 aa, synthetic construct
  REGION 1..475: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 190 - protein, 474 aa, synthetic construct
  REGION 1..474: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 191 - protein, 473 aa, synthetic construct
  REGION 1..473: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 192 - protein, 492 aa, synthetic construct
  REGION 1..492: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 193 - protein, 493 aa, synthetic construct
  REGION 1..493: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 194 - protein, 494 aa, synthetic construct
  REGION 1..494: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 195 - protein, 495 aa, synthetic construct
  REGION 1..495: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 196 - protein, 496 aa, synthetic construct
  REGION 1..496: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 197 - protein, 497 aa, synthetic construct
  REGION 1..497: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 198 - protein, 498 aa, synthetic construct
  REGION 1..498: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 199 - protein, 499 aa, synthetic construct
  REGION 1..499: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWLVLLILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 200 - protein, 481 aa, synthetic construct
  REGION 1..481: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATEHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 201 - protein, 480 aa, synthetic construct
  REGION 1..480: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 202 - protein, 479 aa, synthetic construct
  REGION 1..479: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETAHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 203 - protein, 478 aa, synthetic construct
  REGION 1..478: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 204 - protein, 477 aa, synthetic construct
  REGION 1..477: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVEHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 205 - protein, 476 aa, synthetic construct
  REGION 1..476: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 206 - protein, 475 aa, synthetic construct
  REGION 1..475: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 207 - protein, 474 aa, synthetic construct
  REGION 1..474: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 208 - protein, 473 aa, synthetic construct
  REGION 1..473: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 209 - protein, 472 aa, synthetic construct
  REGION 1..472: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 210 - protein, 471 aa, synthetic construct
  REGION 1..471: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 211 - protein, 470 aa, synthetic construct
  REGION 1..470: note = Description of Artificial Sequence: Synthetic polypeptide
MALPVTALLLPLALLLHAARPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 212 - protein, 434 aa, synthetic construct
  REGION 1..434: note = Description of Artificial Sequence: Synthetic polypeptide
MALPVTALLLPLALLLHAARPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 213 - protein, 447 aa, synthetic construct
  REGION 1..447: note = Description of Artificial Sequence: Synthetic polypeptide
MALPVTALLLPLALLLHAARPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 214 - protein, 430 aa, synthetic construct
  REGION 1..430: note = Description of Artificial Sequence: Synthetic polypeptide
MALPVTALLLPLALLLHAARPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPTRVETHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 215 - protein, 425 aa, synthetic construct
  REGION 1..425: note = Description of Artificial Sequence: Synthetic polypeptide
MALPVTALLLPLALLLHAARPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDPHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 216 - protein, 424 aa, synthetic construct
  REGION 1..424: note = Description of Artificial Sequence: Synthetic polypeptide
MALPVTALLLPLALLLHAARPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSDHLVLGLSAVLGLLLLRWQFPAHYRRLRHALWPSLPDLHRVLGQYLRDTAALSPPKATVSDTCEEVEPSLLEILPKSSERTPLPLLEARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSRSDPKPENPACPWTVLPAGDLPTHDGYLPSNIDDLPSHEAPLADSLEELEPQ

SEQ ID NO: 217 - protein, 105 aa, synthetic construct
  REGION 1..105: note = Description of Artificial Sequence: Synthetic polypeptide
SDPTRVETATETAWISLVTALLLVLGLNAVLGLLLLRKQFPAHYRRLRHALWPSLPDLHRVLGQYLRDTAALSPPRATVSDTCEEVEPSLLEILPRSSERTPLPL

SEQ ID NO: 218 - protein, 105 aa, synthetic construct
  REGION 1..105: note = Description of Artificial Sequence: Synthetic polypeptide
SDPTRVETATETAWISLVTALHLVLGLSAVLGLLLLRWQFPAHYRRLRHALWPSLPDLHRVLGQYLRDTAALSPPRATVSDTCEEVEPSLLEILPRSSERTPLPL

SEQ ID NO: 219 - protein, 98 aa, synthetic construct
  REGION 1..98: note = Description of Artificial Sequence: Synthetic polypeptide
SDPTRVETATETALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 220 - protein, 98 aa, synthetic construct
  REGION 1..98: note = Description of Artificial Sequence: Synthetic polypeptide
SDPTRVETATETALHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 221 - protein, 97 aa, synthetic construct
  REGION 1..97: note = Description of Artificial Sequence: Synthetic polypeptide
SDPTRVETATETLHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 222 - protein, 97 aa, synthetic construct
  REGION 1..97: note = Description of Artificial Sequence: Synthetic polypeptide
SDPTRVETATETLHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 223 - protein, 96 aa, synthetic construct
  REGION 1..96: note = Description of Artificial Sequence: Synthetic polypeptide
SDPTRVETATETHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 224 - protein, 96 aa, synthetic construct
  REGION 1..96: note = Description of Artificial Sequence: Synthetic polypeptide
SDPTRVETATETHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 225 - protein, 109 aa, synthetic construct
  REGION 1..109: note = Description of Artificial Sequence: Synthetic polypeptide
SDPTRVETATETAWILVLISLVTALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 226 - protein, 109 aa, synthetic construct
  REGION 1..109: note = Description of Artificial Sequence: Synthetic polypeptide
SDPTRVETATETAWILVLISLVTALHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 227 - protein, 95 aa, synthetic construct
  REGION 1..95: note = Description of Artificial Sequence: Synthetic polypeptide
SDPTRVETATEHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 228 - protein, 95 aa, synthetic construct
  REGION 1..95: note = Description of Artificial Sequence: Synthetic polypeptide
SDPTRVETATEHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 229 - protein, 92 aa, synthetic construct
  REGION 1..92: note = Description of Artificial Sequence: Synthetic polypeptide
SDPTRVETHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 230 - protein, 92 aa, synthetic construct
  REGION 1..92: note = Description of Artificial Sequence: Synthetic polypeptide
SDPTRVETHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 231 - protein, 87 aa, synthetic construct
  REGION 1..87: note = Description of Artificial Sequence: Synthetic polypeptide
SDPHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 232 - protein, 87 aa, synthetic construct
  REGION 1..87: note = Description of Artificial Sequence: Synthetic polypeptide
SDPHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 233 - protein, 86 aa, synthetic construct
  REGION 1..86: note = Description of Artificial Sequence: Synthetic polypeptide
SDHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 234 - protein, 86 aa, synthetic construct
  REGION 1..86: note = Description of Artificial Sequence: Synthetic polypeptide
SDHLVLGLNAVLGLLLLRKQFPAHYRRLRHALWPSLPDLHRVLGQYLRDTAALSPPRATVSDTCEEVEPSLLEILPRSSERTPLPL

SEQ ID NO: 235 - protein, 55 aa, Homo sapiens
SDPTRVETATETAWISLVTALHLVLGLSAVLGLLLLRWQFPAHYRRLRHALWPSL

SEQ ID NO: 236 - protein, 54 aa, synthetic construct
  REGION 1..54: note = Description of Artificial Sequence: Synthetic polypeptide
SDPTRVETATETWISLVTALHLVLGLSAVLGLLLLRWQFPAHYRRLRHALWPSL

SEQ ID NO: 237 - protein, 53 aa, synthetic construct
  REGION 1..53: note = Description of Artificial Sequence: Synthetic polypeptide
SDPTRVETATETISLVTALHLVLGLSAVLGLLLLRWQFPAHYRRLRHALWPSL

SEQ ID NO: 238 - protein, 54 aa, synthetic construct
  REGION 1..54: note = Description of Artificial Sequence: Synthetic polypeptide
SDPTRVETATETLISLVTALHLVLGLSAVLGLLLLRWQFPAHYRRLRHALWPSL

SEQ ID NO: 239 - protein, 52 aa, synthetic construct
  REGION 1..52: note = Description of Artificial Sequence: Synthetic polypeptide
SDPTRVETATETSLVTALHLVLGLSAVLGLLLLRWQFPAHYRRLRHALWPSL

SEQ ID NO: 240 - protein, 51 aa, synthetic construct
  REGION 1..51: note = Description of Artificial Sequence: Synthetic polypeptide
SDPTRVETATETLVTALHLVLGLSAVLGLLLLRWQFPAHYRRLRHALWPSL

SEQ ID NO: 241 - protein, 52 aa, synthetic construct
  REGION 1..52: note = Description of Artificial Sequence: Synthetic polypeptide
SDPTRVETATETILVTALHLVLGLSAVLGLLLLRWQFPAHYRRLRHALWPSL

SEQ ID NO: 242 - protein, 50 aa, synthetic construct
  REGION 1..50: note = Description of Artificial Sequence: Synthetic polypeptide
SDPTRVETATETVTALHLVLGLSAVLGLLLLRWQFPAHYRRLRHALWPSL

SEQ ID NO: 243 - protein, 49 aa, synthetic construct
  REGION 1..49: note = Description of Artificial Sequence: Synthetic polypeptide
SDPTRVETATETTALHLVLGLSAVLGLLLLRWQFPAHYRRLRHALWPSL

SEQ ID NO: 244 - protein, 48 aa, synthetic construct
  REGION 1..48: note = Description of Artificial Sequence: Synthetic polypeptide
SDPTRVETATETALHLVLGLSAVLGLLLLRWQFPAHYRRLRHALWPSL

SEQ ID NO: 245 - protein, 47 aa, synthetic construct
  REGION 1..47: note = Description of Artificial Sequence: Synthetic polypeptide
SDPTRVETATETLHLVLGLSAVLGLLLLRWQFPAHYRRLRHALWPSL

SEQ ID NO: 246 - protein, 46 aa, synthetic construct
  REGION 1..46: note = Description of Artificial Sequence: Synthetic polypeptide
SDPTRVETATETHLVLGLSAVLGLLLLRWQFPAHYRRLRHALWPSL

SEQ ID NO: 247 - protein, 56 aa, synthetic construct
  REGION 1..56: note = Description of Artificial Sequence: Synthetic polypeptide
SDPTRVETATETAWLISLVTALHLVLGLSAVLGLLLLRWQFPAHYRRLRHALWPSL

SEQ ID NO: 248 - protein, 57 aa, synthetic construct
  REGION 1..57: note = Description of Artificial Sequence: Synthetic polypeptide
SDPTRVETATETAWVLISLVTALHLVLGLSAVLGLLLLRWQFPAHYRRLRHALWPSL

SEQ ID NO: 249 - protein, 58 aa, synthetic construct
  REGION 1..58: note = Description of Artificial Sequence: Synthetic polypeptide
SDPTRVETATETAWLVLISLVTALHLVLGLSAVLGLLLLRWQFPAHYRRLRHALWPSL

SEQ ID NO: 250 - protein, 59 aa, synthetic construct
  REGION 1..59: note = Description of Artificial Sequence: Synthetic polypeptide
SDPTRVETATETAWILVLISLVTALHLVLGLSAVLGLLLLRWQFPAHYRRLRHALWPSL

SEQ ID NO: 251 - protein, 60 aa, synthetic construct
  REGION 1..60: note = Description of Artificial Sequence: Synthetic polypeptide
SDPTRVETATETAWLILVLISLVTALHLVLGLSAVLGLLLLRWQFPAHYRRLRHALWPSL

SEQ ID NO: 252 - protein, 61 aa, synthetic construct
  REGION 1..61: note = Description of Artificial Sequence: Synthetic polypeptide
SDPTRVETATETAWLLILVLISLVTALHLVLGLSAVLGLLLLRWQFPAHYRRLRHALWPSL

SEQ ID NO: 253 - protein, 62 aa, synthetic construct
  REGION 1..62: note = Description of Artificial Sequence: Synthetic polypeptide
SDPTRVETATETAWVLLILVLISLVTALHLVLGLSAVLGLLLLRWQFPAHYRRLRHALWPSL

SEQ ID NO: 254 - protein, 63 aa, synthetic construct
  REGION 1..63: note = Description of Artificial Sequence: Synthetic polypeptide
SDPTRVETATETAWLVLLILVLISLVTALHLVLGLSAVLGLLLLRWQFPAHYRRLRHALWPSL

SEQ ID NO: 255 - protein, 45 aa, synthetic construct
  REGION 1..45: note = Description of Artificial Sequence: Synthetic polypeptide
SDPTRVETATETLVLGLSAVLGLLLLRWQFPAHYRRLRHALWPSL

SEQ ID NO: 256 - protein, 44 aa, synthetic construct
  REGION 1..44: note = Description of Artificial Sequence: Synthetic polypeptide
SDPTRVETATETVLGLSAVLGLLLLRWQFPAHYRRLRHALWPSL

SEQ ID NO: 257 - protein, 43 aa, synthetic construct
  REGION 1..43: note = Description of Artificial Sequence: Synthetic polypeptide
SDPTRVETATETLGLSAVLGLLLLRWQFPAHYRRLRHALWPSL

SEQ ID NO: 258 - protein, 42 aa, synthetic construct
  REGION 1..42: note = Description of Artificial Sequence: Synthetic polypeptide
SDPTRVETATETGLSAVLGLLLLRWQFPAHYRRLRHALWPSL

SEQ ID NO: 259 - protein, 41 aa, synthetic construct
  REGION 1..41: note = Description of Artificial Sequence: Synthetic polypeptide
SDPTRVETATETLSAVLGLLLLRWQFPAHYRRLRHALWPSL

SEQ ID NO: 260 - protein, 45 aa, synthetic construct
  REGION 1..45: note = Description of Artificial Sequence: Synthetic polypeptide
SDPTRVETATEHLVLGLSAVLGLLLLRWQFPAHYRRLRHALWPSL

SEQ ID NO: 261 - protein, 44 aa, synthetic construct
  REGION 1..44: note = Description of Artificial Sequence: Synthetic polypeptide
SDPTRVETATHLVLGLSAVLGLLLLRWQFPAHYRRLRHALWPSL

SEQ ID NO: 262 - protein, 43 aa, synthetic construct
  REGION 1..43: note = Description of Artificial Sequence: Synthetic polypeptide
SDPTRVETAHLVLGLSAVLGLLLLRWQFPAHYRRLRHALWPSL

SEQ ID NO: 263 - protein, 42 aa, synthetic construct
  REGION 1..42: note = Description of Artificial Sequence: Synthetic polypeptide
SDPTRVETHLVLGLSAVLGLLLLRWQFPAHYRRLRHALWPSL

SEQ ID NO: 264 - protein, 41 aa, synthetic construct
  REGION 1..41: note = Description of Artificial Sequence: Synthetic polypeptide
SDPTRVEHLVLGLSAVLGLLLLRWQFPAHYRRLRHALWPSL

SEQ ID NO: 265 - protein, 40 aa, synthetic construct
  REGION 1..40: note = Description of Artificial Sequence: Synthetic polypeptide
SDPTRVHLVLGLSAVLGLLLLRWQFPAHYRRLRHALWPSL

SEQ ID NO: 266 - protein, 39 aa, synthetic construct
  REGION 1..39: note = Description of Artificial Sequence: Synthetic polypeptide
SDPTRHLVLGLSAVLGLLLLRWQFPAHYRRLRHALWPSL

SEQ ID NO: 267 - protein, 38 aa, synthetic construct
  REGION 1..38: note = Description of Artificial Sequence: Synthetic polypeptide
SDPTHLVLGLSAVLGLLLLRWQFPAHYRRLRHALWPSL

SEQ ID NO: 268 - protein, 37 aa, synthetic construct
  REGION 1..37: note = Description of Artificial Sequence: Synthetic polypeptide
SDPHLVLGLSAVLGLLLLRWQFPAHYRRLRHALWPSL

SEQ ID NO: 269 - protein, 36 aa, synthetic construct
  REGION 1..36: note = Description of Artificial Sequence: Synthetic polypeptide
SDHLVLGLSAVLGLLLLRWQFPAHYRRLRHALWPSL

SEQ ID NO: 270 - protein, 35 aa, synthetic construct
  REGION 1..35: note = Description of Artificial Sequence: Synthetic polypeptide
SHLVLGLSAVLGLLLLRWQFPAHYRRLRHALWPSL

SEQ ID NO: 271 - protein, 34 aa, synthetic construct
  REGION 1..34: note = Description of Artificial Sequence: Synthetic polypeptide
HLVLGLSAVLGLLLLRWQFPAHYRRLRHALWPSL

SEQ ID NO: 272 - protein, 244 aa, synthetic construct
  REGION 1..244: note = Description of Artificial Sequence: Synthetic polypeptide
MALPVTALLLPLALLLHAARPSDPTRVETATETAWISLVTALLLVLGLNAVLGLLLLRKQFPAHYRRLRHALWPSLPDLHRVLGQYLRDTAALSPPRATVSDTCEEVEPSLLEILPRSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 273 - protein, 203 aa, synthetic construct
  REGION 1..203: note = Description of Artificial Sequence: Synthetic polypeptide
MALPVTALLLPLALLLHAARPSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 274 - protein, 394 aa, synthetic construct
  REGION 1..394: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALLLVLGLNAVLGLLLLRKQFPAHYRRLRHALWPSLPDLHRVLGQYLRDTAALSPPRATVSDTCEEVEPSLLEILPRSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 275 - protein, 353 aa, synthetic construct
  REGION 1..353: note = Description of Artificial Sequence: Synthetic polypeptide
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVSDPTRVETATETAWISLVTALHLVLGLNAVLGLLLLRKQFPAHYRRLRHALWPSLPDLHRVLGQYLRDTAALSPPRATVSDTCEEVEPSLLEILPRSSERTPLPLLEDEGVAGAPTGSSPQPLQPLSGEDDAYCTFPSRDDLLLFSPSGQGEFRALNARLPLNTDAYLSLQELQGQDPTHLV

(19) United States
(12) Patent Application Publication
(10) Pub. No.: US 2026/0250353 A1
(43) Pub. Date: Aug. 27, 2026

(54) CHIMERIC CYTOKINE RECEPTORS COMPRISING TGF BETA BINDING DOMAINS

(71) Applicant: Allogene Therapeutics, Inc., South San Francisco, CA (US)

(72) Inventors: Regina Junhui LIN, San Mateo, CA (US); Siler PANOWSKI, Berkeley, CA (US); Thomas John VAN BLARCOM, Oakland, CA (US); Shanshan LANG, San Mateo, CA (US); Barbra Johnson SASU, San Francisco, CA (US)

(73) Assignee: Allogene Therapeutics, Inc., South San Francisco, CA (US)

(21) Appl. No.: 19/429,975

(22) Filed: Dec. 22, 2025

Related U.S. Application Data
(63) Continuation of application No. 17/005,757, filed on Aug. 28, 2020, now Pat. No. 12,528,856.
(60) Provisional application No. 62/894,658, filed on Aug. 30, 2019.
(60) Provisional application No. 63/053,322, filed on Jul. 17, 2020.

(51) Int. Cl. C07K 14/71; A61K 40/11; A61K 40/31; A61K 40/42; C12N 5/0783; C12N 15/86

(52) U.S. Cl. CPC C07K 14/71; A61K 40/11; A61K 40/31; A61K 40/4203; A61K 40/4204; A61K 40/4214; C12N 5/0636; C12N 15/86; C07K 2319/03; C07K 2319/70; C12N 2740/15043

(57) ABSTRACT

Provided herein are chimeric cytokine receptors bearing a binding domain capable of binding a TGF-β ligand or a TGF-β receptor antibody. When present on chimeric antigen receptor (CAR)-bearing immune cells (CAR-T-cells), such receptors allow for increased CAR-T cell expansion, activity and persistence, constitutively and/or through engagement of a TGF-β ligand or a TGF-β receptor antibody. Also provided are methods of making and using the chimeric cytokine receptors described herein.

### CROSS REFERENCE TO RELATED APPLICATIONS

The present application is a continuation of U.S. application Ser. No. 17/005,757 filed on Aug. 28, 2020, now U.S. Pat. No. 12,528,856, issued on Jan. 20, 2026, which claims the benefit of priority to U.S. Provisional Application No. 62/894,658, filed on Aug. 30, 2019; and U.S. Provisional Application No. 63/053,322, filed on Jul. 17, 2020, the contents of which are hereby incorporated by reference in their entireties.

### SEQUENCE LISTING

The instant application contains a Sequence Listing which has been submitted electronically in XML file format and is hereby incorporated by reference in its entirety. Said XML copy, created on Mar. 23, 2026, is named AT-030_04US_SL.xml and is 409,886 bytes in size.

### BACKGROUND

Adoptive transfer of immune cells (e.g. T-cells) genetically modified to recognize malignancy-associated antigens is showing promise as a new approach to treating cancer. For example, T-cells can be genetically modified to express chimeric antigen receptors (CARs), which are fusion proteins comprised of an antigen recognition moiety and T-cell activation domains.

T-cell proliferation, cytotoxic potency and persistence is driven by signal transduction pathways. Conventional CAR designs provide two signals—CD3zeta activation (Signal 1) and co-stimulation (Signal 2, e.g. via 4-1BB, OX40, and/or CD28 expression). In some contexts, a third signal (Signal 3), cytokine-induced cytokine receptor signaling (e.g. cytokine support for immune potentiation), may be desirable. Approaches to provide Signal 3 have however been met with significant limitations.

One approach to provide cytokine support includes combining CAR-T-cell therapy with systemic infusions of recombinant cytokines/cytokine mimetics, and engineering CAR-T-cells to secrete/express cytokines extracellularly. As cytokines have pleiotropic effects and can also impact the function of other cell types, the systemic administration or production of immune-potentiating cytokines by CAR-T-cells have at least two major drawbacks: (i) these approaches can cause systemic toxicity in humans, and (ii) in the context of allogeneic CAR-T-cell therapy, these approaches may cause bystander host immune-activation that could accelerate the rejection of allogeneic CAR-T-cells, thereby compromising therapeutic efficacy. Another approach to provide cytokine support was based on introducing a constitutively activated dimerized cytokine receptor, an IL-7Ra—this limits the nature (IL-7 signaling only) and magnitude of signaling output. Yet another approach to provide cytokine support involved incorporating Signal 3 directly into the CAR molecule (Nat Med. 2018 March; 24(3):352-359.). A limitation of this approach is that the strength of Signal 3 is dependent on the strength of CAR activation. In the absence of target (and CAR activation), Signal 3 would not be transduced.

Needed are solutions to circumvent these drawbacks by targeting cytokine signals specifically to CAR-T-cells in a context-dependent manner, thus allowing for an improved safety profile and therapeutic efficacy. Provided herein and compositions and methods that address this need.

### SUMMARY

Provided herein are chimeric cytokine receptors comprising TGF-β binding domains. Provided herein are inducible TGF-β-driven chimeric cytokine receptors, active when engaged with a ligand of the transforming growth factor beta cytokine family (TGF-β ligands, e.g., TGF-β1, TGF-β2, and TGF-β3) or activation with an anti-TGF-β-receptor antibody. When present on chimeric antigen receptor (CAR)-bearing immune cells, and engaged with TGF-β ligands and/or activation with an anti-TGF-βR antibody, such receptors allow for increased cytokine receptor signaling (Signal 3), leading to increased immune cell activation, proliferation, persistence, and/or potency of the CAR-bearing immune cells. Accordingly, the chimeric cytokine receptors of the disclosure allow for cytokine signals to be transmitted into the immune cell with endogenous TGF-β ligands, whereby blocking their immune-suppressive signals, and converting them into immune-potentiating signals that can work in concert with, or synergize, CAR-driven activity. Moreover, as clinically approved anti-TGF-β receptor antibodies can cluster and activate the chimeric cytokine receptors of the disclosures, patients treated with anti-TGF-β receptor may benefit not only from the blockage of the endogenous TGF-β signaling, but from also the activation of cytokine signaling in cells bearing the chimeric cytokine receptors. Also provided herein are constitutively active TGF-β-driven of TGF-β binding domain-containing chimeric cytokine receptors; such receptors continue to signal in the absence of an inducer, but can be further induced or can exhibit further improved properties or activities, for example, in the presence of a TGF-β ligand or an anti-TGF-βR antibody. In some embodiments, the TGF-βR is TGF-βR2, and the antibody is an anti-TGF-βR2 antibody. As used herein, “TGF-beta” is used interchangeably with “TGF-β.”

Accordingly, in one aspect, provided herein is a chimeric cytokine receptor comprising: (a) a binding domain comprising an extracellular portion of a TGF-β receptor, or a TGF-β antigen binding domain; (b) a transmembrane domain; (c) a Janus Kinase (JAK)-binding domain; and (d) a recruiting domain. As used herein, “extracellular portion” refers to any portion of an extracellular domain of a TGF-β receptor.

In a related aspect provided herein is a polynucleotide encoding any one of the chimeric cytokine receptors of the disclosure, and an expression vector comprising such a polynucleotide. In some embodiments, the polynucleotide further encodes for a chimeric antigen receptor (CAR), wherein the CAR binds to a target of interest. The target of interest can be any molecule of interest, including, for example, without limitation any one or more of those presented in Table 8.

In a further aspect, provided herein is an engineered immune cell comprising at least one chimeric cytokine receptor of the disclosure. In another aspect, provided herein is an engineered immune cell comprising at least one chimeric antigen receptor (CAR) and at least one chimeric cytokine receptor of the disclosure. In some embodiments the immune cell is a T-cell. In some embodiments the immune cell is an allogeneic immune cell. In other embodiments, the immune cell is an autologous immune cell. The immune cell may be selected from the group consisting of: T-cell, dendritic cell, killer dendritic cell, mast cell, NK-cell, macrophage, monocyte, B-cell and an immune cell derived from a stem cell. In a related aspect, provided herein is a pharmaceutical composition comprising any of the engineered immune cells of the disclosure, and a kit comprising such a pharmaceutical composition. Also provided herein is a method of making the immune cell.

In another aspect, provided herein is a method of treating a cancer in a subject, comprising administering to the subject a therapeutically effective amount of any of the engineered immune cells described herein.

### BRIEF DESCRIPTION OF THE DRAWINGS

FIG. 1 shows a schematic of the inducible chimeric cytokine receptor of the disclosure.

FIG. 2A shows a schematic of the lentiviral vector used to co-express the dominant negative truncations of the TGFβR1 or TGFβR2 cytokine receptor with the 2^{nd} generation EGFRvIII CAR.

FIG. 2B shows the inhibition of the TGF-β signaling by expression of either the TGFβR1 DN or TGFβR2 DN.

FIG. 3 shows a general schematic of the lentiviral vector used to co-express the TGFβR2 cytokine receptor with the 2^{nd} generation EGFRvIII CAR.

FIG. 4A shows a schematic of the prototypic lentiviral vector used, bearing the IL7R(316-459) and the IL12Rb2(775-825) recruiting domains to mimic IL7 and IL12 signaling in CAR-T-cells.

FIG. 4B shows TGF-β signaling activity determined by a luciferase reporter assay.

FIG. 4C shows the activation of the chimeric cytokine receptors of FIG. 4A in the presence of TGF-β.

FIG. 5A shows a schematic of the prototypic lentiviral vector used, having truncations in the binding domain.

FIG. 5B shows TGF-β signaling activity determined by a luciferase reporter assay.

FIG. 5C shows the activation of the chimeric cytokine receptors of FIG. 5A in the presence of TGF-β.

FIG. 6A shows a schematic of the modification introduced into the TGFβR2 cassette.

FIG. 6B shows that the TGFβR2ΔN25 chimeric cytokine receptors still retained the ability to inhibit TGF-β signaling.

FIG. 6C shows the activation of the chimeric cytokine receptors of FIG. 6A, evaluated by the STAT reporter activity.

FIG. 7 shows a schematic of the constitutively active chimeric cytokine receptor.

FIG. 8A shows a schematic of the modification introduced into the TGFβR2 cassette.

FIG. 8B shows the inhibition of TGF-β signaling by the expression of the chimeric cytokine receptors of FIG. 8A.

FIG. 8C shows the activation of cytokine signaling via the chimeric cytokine receptors of FIG. 8A, measured by the STAT5 reporter activity.

FIGS. 9A-9B show the amino acid sequences for the wild type TPOR and the various transmembrane deletion or insertion variants. FIGS. 9A-B disclose SEQ ID NOS 235-246, 235, and 247-254, respectively, in order of appearance.

FIG. 10A shows the amino acid sequences for the wild type TPOR and additional transmembrane variants. FIG. 10B shows the inhibition of endogenous TGF-β signaling as determined by luciferase assay by the overexpression of TGF-β-driven chimeric cytokine receptors shown in FIG. 10A, in the presence of different concentrations of TGF-β. FIG. 10C shows the activation of chimeric cytokine receptors in the presence of TGF-β at various concentrations. FIG. 10A discloses SEQ ID NOS 235 and 255-271, respectively, in order of appearance.

FIG. 11A shows schematics of chimeric cytokine receptor (CCR) CAR expression construct where the expression of the CCR and the EGFRvIII CAR are linked by a P2A peptide. FIGS. 11B-11C are bar graphs depicting the yield of CAR+ T cells expressing various CCRs. FIGS. 11D-11E show results of STAT5 phosphorylation in CAR T cells expressing various CCR or controls. FIGS. 11F-11G depict CAR T cells phenotype at Day 14 of production. FIG. 11H shows results of total TGF-βR2 extracellular staining on CAR T cells. FIG. 11I depicts results of inhibition of TGF-βR2 signaling in CAR T cells expressing different CCRs in the presence of different concentrations of TGF-β.

FIG. 12A exhibits results of cytotoxicity assay of CAR T cells expressing various CCRs against U87-EGFRvIII cells in the absence of exogenous TGFβ. FIGS. 12B-12C show results of cytotoxicity assay of CAR T cells expressing various CCRs at different concentrations of TGFβ.

FIG. 13A shows STAT5 phosphorylation and FIG. 13B depicts the T cell phenotype of CAR T cells expressing CCRs with the S505N/W515K with or without the K553R/K573R substitutions in the TOPR/MPLR and JAK binding domain. All TGFbR2 chimeric cytokine receptor constructs tested in this experiment contain the S505N/W515K substitutions. The constructs labeled RR further contain the additional K553R/K573R substitutions.

FIGS. 14A-14B show results of long-term cell killing assay of CAR T cells expressing various CCRs in the absence (FIG. 14A) or presence (FIG. 14B) of 5 ng/ml TGFβ.

FIGS. 15A-15C show results of activation of STAT5 signaling (FIG. 15A), inhibition of TGFβ signaling (FIG. 15B), and long-term cell killing assay (FIG. 15C) of CAR T cells expressing various CCRs, some of which have reduced affinity for TGFβ (e.g., D32A, E119A and/or I53A substitutions in the ECD of TGFβR2).

FIGS. 16A-16B compare the effects of TGFβR2 chimeric cytokine receptors with or without the degradation-resistant K533R/K573R substitutions on STAT5 signaling (FIG. 16A) and long-term cell killing (FIG. 16B).

### DETAILED DESCRIPTION

Provided herein are chimeric cytokine receptors comprising TGF-β binding domains. Provided herein are inducible chimeric cytokine receptors, active when engaged with TGF-β ligands (e.g. TGF-β1, TGF-β2, and/or TGF-β3) or activation with an anti-TGF-β-receptor antibody. Also provided herein are constitutively active chimeric cytokine receptors comprising TGF-β binding domains. Also provided herein are chimeric antigen receptor (CAR)-bearing immune cells (CAR-I-cells, e.g. CAR-T-cells), expressing the chimeric cytokine receptors of the disclosure. In some embodiments, the constitutively active chimeric cytokine receptors exhibit improved properties or activities when engaged with a TGF-β ligand or activation with an anti-TGF-β-receptor antibody, as compared with constitutively active chimeric cytokine receptors without a TGF-β binding domain. Also provided herein are methods of making and using the chimeric cytokine receptors.

### I. TGF-β-Bearing Chimeric Cytokine Receptors

The chimeric cytokine receptors of the disclosure activate signaling upon binding of a TGF-β ligand (for example, TGF-β1, TGF-β2, and/or TGF-β3), or an anti-TGF-β-receptor antibody. These receptors activate signaling when monomers of the receptor cluster and/or dimerize. The chimeric cytokine receptors of the disclosure are dual-function chimeric cytokine receptors which can simultaneously neutralize the immune-suppressive effects of a TGF-β ligand, and mimic the transmission of an immune-potentiating cytokine signal.

In some embodiments, a monomer of the chimeric cytokine receptor of the disclosure comprises: (a) a binding domain capable of binding a TGF-β ligand or an anti-TGF-β-receptor antibody; (b) a transmembrane domain; (c) a Janus Kinase (JAK)-binding domain; and; (d) a STAT-recruiting domain (e.g. from the cytoplasmic domain of a receptor; e.g. from a cytokine receptor). Each domain can be linked either directly or via one or more peptide linkers. In some embodiments, a monomer of the chimeric cytokine receptor of the disclosure comprises: (a) a binding domain capable of binding a TGF-β ligand or an anti-TGF-β-receptor antibody; (b) a transmembrane domain; (c) a Janus Kinase (JAK)-binding domain; and; (d) a recruiting domain (e.g. from the cytoplasmic domain of a receptor; e.g. from a cytokine receptor). The recruiting domain can be a STAT-recruiting domain, an AP1—recruiting domain, a Myc/Max recruiting domain; or a NFkB-recruiting domain. In some embodiments, the chimeric cytokine receptors are clustered and activated when they bind to TGF-β ligands, and/or are clustered and activated with an anti-TGF-β-receptor antibody. The chimeric cytokine receptors activate signaling upon for example binding a TGF-β ligand, and/or a TGF-β-receptor antibody. In some embodiments, the TGF-β receptor antibody is, without limitation, PF-03446962 or LY3022859. In some embodiments, the chimeric cytokine receptors are constitutively clustered or dimerized.

As used herein, “TGF-β ligand,” refers to TGF-β1, TGF-β2, and TGF-β3, and isoforms and derivatives thereof. It should be understood that “TGF-β ligand” and “TGF-β” are used interchangeably herein.

### A. Binding Domains

The chimeric cytokine receptors of the disclosure comprise a binding domain capable of binding a TGF-β ligand or an anti-TGF-β-receptor antibody. As referred to herein, a binding domain is the domain of the chimeric cytokine receptor that extends into the extracellular space. The binding domain binds and sequesters TGF-β away from the endogenous TGF-β receptor, thereby preventing or reducing TGF-β-induced immune-suppression. The binding domains of the disclosure bind with TGF-β ligands and anti-TGF-β-receptor antibodies, leading to binding-induced signal transduction.

In some embodiments, the binding domain comprises an extracellular portion of a TGF-β receptor, for example the extracellular portion of TGFβR1 or TGFβR2.

In some embodiments, the binding domain comprises an extracellular portion of a wild type TGFβ receptor. In some embodiments, the TGF-β receptor comprises one or more mutations that enhance or alter the affinity to the binding to the TGFβ ligands.

In some embodiments, the binding domain comprises the extracellular portion of a wild type TGFβR1 or TGFβR2; in some embodiments, the binding domain comprises the extracellular portion of a wild type TGFβR1 or TGFβR2 and comprises the amino acid sequence of SEQ ID NOS: 2 or 3, respectively.

In some embodiments, the binding domain comprises mutations to the extracellular portion of a wild type TGF-β receptor. In some embodiments, the binding domain comprises mutations to the extracellular portion of a wild type TGF-β receptor, and comprises the amino acid sequences of any one of SEQ ID NO: 4 to SEQ ID NO: 20. In some embodiments, the chimeric cytokine receptor comprises a binding domain that is at least 80%, 85%, 90%, 95%, 98%, or 99%, or 100% identical to any one of the amino acid sequences of SEQ ID NOs: 4-20. In some embodiments, the binding domain does not comprise a signal sequence.

Table 1 shows exemplary binding domain amino acid sequences of the disclosure. It is noted that the expression and extracellular location of the exemplary binding domain sequences, such as TGF-β receptor amino acid sequences, can be achieved with the use of a signal sequence. In an exemplary embodiment, a CD8 signal sequence (CD8SS) MALPVTALLLPLALLLHAARP (SEQ ID NO: 1) is utilized. In some embodiments, the binding domain comprises the extracellular domain of wild type TGFβR2 comprising the amino acid sequence of SEQ ID NO: 159. In some embodiments, the signal sequence is the endogenous signal sequence of human TGF-βR2.

TABLE 1
Exemplary Binding Domain Sequences

| | | SEQ |
|---|---|---|
| Binding Domain | Amino acid sequence | ID NO: |
| TGFβR1 (1-126) | MEAAVAAPRPRLLLLVLAAAAAAAAALLPGAT | 2 |
| | ALQCFCHLCTKDNFTCVTDGLCFVSVTETTDKVI | |
| | HNSMCIAEIDLIPRDRPFVCAPSSKTGSVTTTYCC | |
| | NQDHCNKIELPTTVKSSPGLGPVEL | |
| TGFβR2 (1-166) | MGRGLLRGLWPLHIVLWTRIASTIPPHVQKSVNN | 3 |
| Underlined TGFβR2 | DMIVTDNNGAVKFPQLCKFCDVRFSTCDNQKSC | |
| signal sequence | MSNCSITSICEKPQEVCVAVWRKNDENITLETVC | |
| | HDPKLPYHDFILEDAASPKCIMKEKKKPGETFFM | |
| | CSCSSDECNDNIIFSEEYNTSNPDLLLVIFQ | |
| TGFβR2ΔN25 | QLCKFCDVRFSTCDNQKSCMSNCSITSICEKPQE | 4 |
| | VCVAVWRKNDENITLETVCHDPKLPYHDFILED | |
| | AASPKCIMKEKKKPGETFFMCSCSSDECNDNIIFS | |
| | EEYNTSNPDLLLVIFQ | |
| TGFβR2ΔN25FSE-N8 | QLCKFCDVRFSTCDNQKSCMSNCSITSICEKPQE | 5 |
| | VCVAVWRKNDENITLETVCHDPKLPYHDFILED | |
| | AASPKCIMKEKKKPGETFFMCSCSSDECNDNIIFS | |
| | ELLLVIFQ | |
| TGFβR2ΔN25FSE-N6 | QLCKFCDVRFSTCDNQKSCMSNCSITSICEKPQE | 6 |
| | VCVAVWRKNDENITLETVCHDPKLPYHDFILED | |
| | AASPKCIMKEKKKPGETFFMCSCSSDECNDNIIFS | |
| | EPDLLLVIFQ | |
| TGFβR2ΔN25FSE-N5 | QLCKFCDVRFSTCDNQKSCMSNCSITSICEKPQE | 7 |
| | VCVAVWRKNDENITLETVCHDPKLPYHDFILED | |
| | AASPKCIMKEKKKPGETFFMCSCSSDECNDNIIFS | |
| | EEPDLLLVIFQ | |
| TGFβR2ΔN25FSE-N4 | QLCKFCDVRFSTCDNQKSCMSNCSITSICEKPQE | 8 |
| | VCVAVWRKNDENITLETVCHDPKLPYHDFILED | |
| | AASPKCIMKEKKKPGETFFMCSCSSDECNDNIIFS | |
| | EEYPDLLLVIFQ | |
| TGFβR2ΔN25FSE-N3 | QLCKFCDVRFSTCDNQKSCMSNCSITSICEKPQE | 9 |
| | VCVAVWRKNDENITLETVCHDPKLPYHDFILED | |
| | AASPKCIMKEKKKPGETFFMCSCSSDECNDNIIFS | |
| | EEYNPDLLLVIFQ | |
| TGFβR2ΔN25.Glyc33 | QLCKFCDNRTSTCDNQKSCMSNCSITSICEKPQE | 10 |
| | VCVAVWRKNDENITLETVCHDPKLPYHDFILED | |
| | AASPKCIMKEKKKPGETFFMCSCSSDECNDNIIFS | |
| | EEYNTSNPDLLLVIFQ | |
| TGFβR2ΔN25.Glyc40 | QLCKFCDVRFSTCDNQTSCMSNCSITSICEKPQEV | 11 |
| | CVAVWRKNDENITLETVCHDPKLPYHDFILEDA | |
| | ASPKCIMKEKKKPGETFFMCSCSSDECNDNIIFSE | |
| | EYNTSNPDLLLVIFQ | |
| TGFβR2ΔN25.Glyc56 | QLCKFCDVRFSTCDNQKSCMSNCSITSICENPTEV | 12 |
| | CVAVWRKNDENITLETVCHDPKLPYHDFILEDA | |
| | ASPKCIMKEKKKPGETFFMCSCSSDECNDNIIFSE | |
| | EYNTSNPDLLLVIFQ | |
| TGFβR2ΔN25.Glyc58 | QLCKFCDVRFSTCDNQKSCMSNCSITSICEKPNET | 13 |
| | CVAVWRKNDENITLETVCHDPKLPYHDFILEDA | |
| | ASPKCIMKEKKKPGETFFMCSCSSDECNDNIIFSE | |
| | EYNTSNPDLLLVIFQ | |
| TGFβR2ΔN25.V33K | QLCKFCDKRFSTCDNQKSCMSNCSITSICEKPQE | 14 |
| | VCVAVWRKNDENITLETVCHDPKLPYHDFILED | |
| | AASPKCIMKEKKKPGETFFMCSCSSDECNDNIIFS | |
| | EEYNTSNPDLLLVIFQ | |
| TGFβR2ΔN25.E70K | QLCKFCDVRFSTCDNQKSCMSNCSITSICEKPQE | 15 |
| | VCVAVWRKNDKNITLETVCHDPKLPYHDFILED | |
| | AASPKCIMKEKKKPGETFFMCSCSSDECNDNIIFS | |
| | EEYNTSNPDLLLVIFQ | |
| TGFβR2ΔN25.Glyc87 | QLCKFCDVRFSTCDNQKSCMSNCSITSICEKPQE | 16 |
| | VCVAVWRKNDENITLETVCHDPKLPYHNFTLED | |
| | AASPKCIMKEKKKPGETFFMCSCSSDECNDNIIF | |
| TGFβR2ΔN25.Glyc89 | QLCKFCDVRFSTCDNQKSCMSNCSITSICEKPQE | 17 |
| | VCVAVWRKNDENITLETVCHDPKLPYHDFNLTD | |
| | AASPKCIMKEKKKPGETFFMCSCSSDECNDNIIF | |
| TGFβR2ΔN25.Glyc102 | QLCKFCDVRFSTCDNQKSCMSNCSITSICEKPQE | 18 |
| | VCVAVWRKNDENITLETVCHDPKLPYHDFILED | |
| | AASPKCIMKNKTKPGETFFMCSCSSDECNDNIIF | |
| TGFβR2ΔN25.Glyc123 | QLCKFCDVRFSTCDNQKSCMSNCSITSICEKPQE | 19 |
| | VCVAVWRKNDENITLETVCHDPKLPYHDFILED | |
| | AASPKCIMKEKKKPGETFFMCSCSSDECNDNITF | |
| TGFβR2ΔN25.I89E | QLCKFCDVRFSTCDNQKSCMSNCSITSICEKPQE | 20 |
| | VCVAVWRKNDENITLETVCHDPKLPYHDFELED | |
| | AASPKCIMKEKKKPGETFFMCSCSSDECNDNIIF | |
| TGFβR2 (without the | IPPHVQKSVNNDMIVTDNNGAVKFPQLCKFCDV | 159 |
| signal sequence) | RFSTCDNQKSCMSNCSITSICEKPQEVCVAVWRK | |
| | NDENITLETVCHDPKLPYHDFILEDAASPKCIMKE | |
| | KKKPGETFFMCSCSSDECNDNIIFSEEYNTSNPDL | |
| | LLVIFQ | |

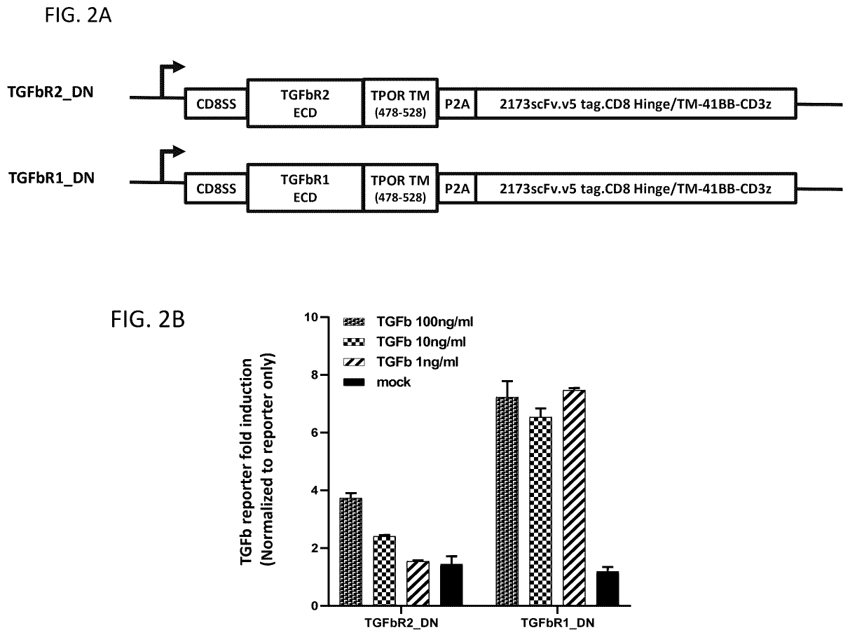

In some embodiments, the chimeric cytokine receptor is a dominant negative (DN) wherein the binding domain of the TGF-β receptor is expressed, but the chimeric cytokine receptor does not comprise an intracellular signaling domain—the chimeric cytokine receptor can bind TGF-β but does not transmit a positive signal (DN chimeric cytokine receptor). In some embodiments, the TGF-β receptor is TGFβR1 (dominant-negative TGFβR1, or TGFβR1 DN) and comprises the amino acid sequence of SEQ ID NO: 2. In some embodiments, the TGF-β receptor is TGFβR2 (dominant-negative TGFβR2, or TGFβR2 DN) and comprises the amino acid sequence of SEQ ID NO: 3. TGF-β receptor dominant negative sequences may be expressed with the aid of a signal sequence, e.g. a CD8SS signal sequence of SEQ ID NO: 1. Example schematics of a DN chimeric cytokine receptor are shown in FIG. 2A.

In other embodiments, the binding domain comprises a TGF-β antigen binding domain. Such antigen binding domains include, but are not limited to, a single chain variable fragment (scFv) that can bind the TGF-β ligands, and single domain antibodies (nanobodies). These scFvs and single domain antibodies may include commercially available scFvs and single domain antibodies, and those derived from, for example, camelid and shark antibodies.

In other embodiments, the binding domain comprises a TGF-β antigen binding domain, wherein the antigen binding domain comprises a Fab fragment.

### B. Transmembrane Domains

The chimeric cytokine receptors of the disclosure comprise transmembrane domains. Such transmembrane domains are coupled to the extracellular binding domain on the N-terminus, and to additional intracellular/cytoplasmic domains on the C-terminus. In some embodiments, the coupling is achieved optionally through a linker.

As used herein, the transmembrane domains are capable of insertion into the membrane of a cell in which it is expressed. In some embodiments, the transmembrane domains of the disclosure span a cellular membrane, and comprise an extracellular portion, and/or an intracellular portion.

In some embodiments, the transmembrane domains of the disclosure are engineered and do not resemble any naturally occurring transmembrane domain, e.g. they are non-naturally occurring.

In other embodiments, the transmembrane domains of the disclosure are derived from naturally occurring receptors.

In some embodiments, the transmembrane and/or JAK domains of the disclosure are derived from, for example, one or more of the following receptors: erythropoietin receptor (EpoR), Interleukin 6 signal transducer (GP130 or IL6ST), prolactin receptor (PrlR), growth hormone receptor (GHR), granulocyte colony-stimulating factor receptor (GCSFR), and thrombopoietin receptor/myeloproliferative leukemia protein receptor (TPOR/MPLR). When derived from naturally occurring receptors, the entire receptor, or the entire transmembrane sequence of the receptor may not be necessary to effectuate constitutive activation and constitutive JAK binding/activation on the intracellular portion. Accordingly fragments of naturally occurring receptors may be utilized. Furthermore, certain mutations may be introduced into the transmembrane domains derived from naturally occurring receptors, to further tune the downstream JAK-dependent signaling. In some embodiments, the chimeric cytokine receptor of the disclosure comprises a portion or a fragment of a naturally occurring receptor, e.g., the transmembrane and/or JAK binding/activation domain of the naturally occurring receptor, optionally comprising one or more mutations therein (e.g., one or more deletions, insertions and/or substitutions).

In some embodiments, the transmembrane and/or JAK domains of the disclosure is derived from the naturally occurring EpoR receptor.

In some embodiments, the transmembrane and/or JAK domains of the disclosure is derived from the naturally occurring GP130 receptor.

In some embodiments, the transmembrane and/or JAK domains of the disclosure is derived from the naturally occurring PrlR receptor.

In some embodiments, the transmembrane and/or JAK domains of the disclosure is derived from the naturally occurring GHR receptor.

In some embodiments, the transmembrane and/or JAK domains of the disclosure is derived from the naturally occurring GCSF receptor.

In some embodiments, the transmembrane and/or JAK domains of the disclosure is derived from the naturally occurring TPOR receptor. When the TPOR transmembrane domain assumes a permissive homodimeric conformation, such as in response to a ligand or forced activation resulting from the introduction of engineered modifications, it is capable of activating downstream cytokine signaling in a JAK2-dependent fashion. The introduction of various modifications to the TPOR transmembrane domain can result in the following: the immune-potentiating cytokine signal may either be (a) quiescent until induced to activate in the presence of extracellular TGF-β, or (b) constitutively active regardless of TGF-β availability.

Table 2 provides exemplary full length sequences of naturally occurring receptors provided in the disclosure, from which the transmembrane and/or JAK domains are derived.

TABLE 2
Exemplary Naturally Occurring Receptors

| | SEQ |
|---|---|
| | ID |
| Naturally Occurring Receptor Name | NO: |
| >AAI12154.1 Erythropoietin receptor [Homo sapiens] | 21 |
| MDHLGASLWPQVGSLCLLLAGAAWAPPPNLPDPKFESKAALLAARGPEELLCFTE | |
| RLEDLVCFWEEAASAGVGPGNYSFSYQLEDEPWKLCRLHQAPTARGAVRFWCSLP | |
| TADTSSFVPLELRVTAASGAPRYHRVIHINEVVLLDAPVGLVARLADESGHVVLR | |
| WLPPPETPMTSHIRYEVDVSAGNGAGSVQRVEILEGRTECVLSNLRGRTRYTFAV | |
| RARMAEPSFGGFWSAWSEPVSLLTPSDLDPLILTLSLILVVILVLLTVLALLSHR | |
| RALKQKIWPGIPSPESEFEGLFTTHKGNFQLWLYQNDGCLWWSPCTPFTEDPPAS | |
| LEVLSERCWGTMQAVEPGTDDEGPLLEPVGSEHAQDTYLVLDKWLLPRNPPSEDL | |
| PGPGGSVDIVAMDEGSEASSCSSALASKPSPEGASAASFEYTILDPSSQLLRPWT | |
| LCPELPPTPPHLKYLYLVVSDSGISTDYSSGDSQGAQGGLSDGPYSNPYENSLIP | |
| AAEPLPPSYVACS | |
| >AAI17403.1 Interleukin 6 signal transducer | 22 |
| (GP130, oncostatin M receptor) [Homo sapiens] | |
| MLTLQTWLVQALFIFLTTESTGELLDPCGYISPESPVVQLHSNFTAVCVLKEKCM | |
| DYFHVNANYIVWKTNHFTIPKEQYTIINRTASSVTFTDIASLNIQLTCNILTFGQ | |
| LEQNVYGITIISGLPPEKPKNLSCIVNEGKKMRCEWDRGRETHLETNFTLKSEWA | |
| THKFADCKAKRDTPTSCTVDYSTVYFVNIEVWVEAENALGKVTSDHINFDPVYKV | |
| KPNPPHNLSVINSEELSSILKLTWTNPSIKSVIILKYNIQYRTKDASTWSQIPPE | |
| DTASTRSSFTVQDLKPFTEYVFRIRCMKEDGKGYWSDWSEEASGITYEDRPSKAP | |
| SFWYKIDPSHTQGYRTVQLVWKTLPPFEANGKILDYEVTLTRWKSHLQNYTVNAT | |
| KLTVNLTNDRYVATLTVRNLVGKSDAAVLTIPACDFQATHPVMDLKAFPKDNMLW | |
| VEWTTPRESVKKYILEWCVLSDKAPCITDWQQEDGTVHRTYLRGNLAESKCYLIT | |
| VTPVYADGPGSPESIKAYLKQAPPSKGPTVRTKKVGKNEAVLEWDQLPVDVQNGF | |
| IRNYTIFYRTIIGNETAVNVDSSHTEYTLSSLTSDTLYMVRMAAYTDEGGKDGPE | |
| FTFTTPKFAQGEIEAIVVPVCLAFLLTTLLGVLFCFNKRDLIKKHIWPNVPDPSK | |
| SHIAQWSPHTPPRHNFNSKDQMYSDGNFTDVSVVEIEANDKKPFPEDLKSLDLFK | |
| KEKINTEGHSSGIGGSSCMSSSRPSISSSDENESSQNTSSTVQYSTVVHSGYRHQ | |
| VPSVQVFSRSESTQPLLDSEERPEDLQLVDHVDGGDGILPRQQYFKQNCSQHESS | |
| PDISHFERSKQVSSVNEEDFVRLKQQISDHISQSCGSGQMKMFQEVSAADAFGPG | |
| TEGQVERFETVGMEAATDEGMPKSYLPQTVRQGGYMPQ | |
| >XP_011512371.1 prolactin receptor isoform X2 [Homo | 23 |
| sapiens] | |
| MKENVASATVFTLLLFLNTCLLNGQLPPGKPEIFKCRSPNKETFTCWWRPGTDGG | |
| LPTNYSLTYHREGETLMHECPDYITGGPNSCHFGKQYTSMWRTYIMMVNATNQMG | |
| SSFSDELYVDVTYIV0PDPPLELAVEVK0PEDRKPYLWIKWSPPTLIDLKTGWFT | |
| LLYEIRLKPEKAAEWEIHFAGQQTEFKILSLHPGQKYLVQVRCKPDHGYWSAWSP | |
| ATFIQIPSDFTMNDTTVWISVAVLSAVICLIIVWAVALKGYSMVTCIFPPVPGPK | |
| IKGFDAHLLEKGKSEELLSALGCQDFPPTSDYEDLLVEYLEVDDSEDQHLMSVHS | |
| KEHPSQGMKPTYLDPDTDSGRGSCDSPSLLSEKCEEPQANPSTFYDPEVIEKPEN | |
| PETTHTWDPQCISMEGKIPYFHAGGSKCSTWPLPQPSQHNPRSSYHNITDVCELA | |
| VGPAGAPATLLNEAGKDALKSSQTIKSREEGKATQQREVESFHSETDQDTPWLLP | |
| QEKTPFGSAKPLDYVEIHKVNKDGALSLLPKQRENSGKPKKPGTPENNKEYAKVS | |
| GVMDNNILVLVPDPHAKNVACFEESAKEAPPSLEQNQAEKALANFTATSSKCRLQ | |
| LGGLDYLDPACFTHSFH | |
| >NP_000154.1 growth hormone receptor isoform 1 | 24 |
| precursor [Homo sapiens] | |
| MDLWQLLLTLALAGSSDAFSGSEATAAILSRAPWSLQSVNPGLKTNSSKEPKFTK | |
| CRSPERETFSCHWTDEVHHGTKNLGPIQLFYTRRNTQEWTQEWKECPDYVSAGEN | |
| SCYFNSSFTSIWIPYCIKLTSNGGTVDEKCFSVDEIVQPDPPIALNWTLLNVSLT | |
| GIHADIQVRWEAPRNADIQKGWMVLEYELQYKEVNETKWKMMDPILTTSVPVYSL | |
| KVDKEYEVRVRSKQRNSGNYGEFSEVLYVTLPQMSQFTCEEDFYFPWLLIIIFGI | |
| FGLTVMLFVFLFSKQQRIKMLILPPVPVPKIKGIDPDLLKEGKLEEVNTILAIHD | |
| SYKPEFHSDDSWVEFIELDIDEPDEKTEESDTDRLLSSDHEKSHSNLGVKDGDSG | |
| RTSCCEPDILETDFNANDIHEGTSEVAQPQRLKGEADLLCLDQKNQNNSPYHDAC | |
| PATQQPSVIQAEKNKPQPLPTEGAESTHQAAHIQLSNPSSLSNIDFYAQVSDITP | |
| AGSVVLSPGQKNKAGMSQCDMHPEMVSLCQENFLMDNAYFCEADAKKCIPVAPHI | |
| KVESHIQPSLNQEDIYITTESLTTAAGRPGTGEHVPGSEMPVPDYTSIHIVQSPQ | |
| GLILNATALPLPDKEFLSSCGYVSTDQLNKIMP | |
| >XP_016855859.1 granulocyte colony-stimulating factor | 25 |
| receptor isoform X1 [Homo sapiens] | |
| MARLGNCSLTWAALIILLLPGSLEECGHISVSAPIVHLGDPITASCIIKQNCSHL | |
| DPEPQILWRLGAELQPGGRQQRLSDGTQESIITLPHLNHTQAFLSCCLNWGNSLQ | |
| ILDQVELRAGYPPAIPHNLSCLMNLTTSSLICQWEPGPETHLPTSFTLKSFKSRG | |
| NCQTQGDSILDCVPKDGQSHCCIPRKHLLLYQNMGIWVQAENALGTSMSPQLCLD | |
| PMDVVKLEPPMLRTMDPSPEAAPPQAGCLQLCWEPWQPGLHINQKCELRHKPQRG | |
| EASWALVGPLPLEALQYELCGLLPATAYTLQIRCIRWPLPGHWSDWSPSLELRTT | |
| ERAPTVRLDTWVVRQRQLDPRTVQLFWKPVPLEEDSGRIQGYVVSWRPSGQAGAI | |
| LPLCNTTELSCTFHLPSEAQEVALVAYNSAGTSRPTPVVFSESRGPALTRLHAMA | |
| RDPHSLWVGWEPPNPWPQGYVIEWGLGPPSASNSNKTWRMEQNGRATGFLLKENI | |
| RPFQLYEIIVTPLYQDTMGPSQHVYAYSQEMAPSHAPELHLKHIGKTWAQLEWVP | |
| EPPELGKSPLTHYTIFWTNAQNQSFSAILNASSRGFVLHGLEPASLYHIHLMAAS | |
| QAGATNSTVLTLMTLTPEGSELHIILGLFGLLLLLTCLCGTAWLCCSPNRKNPLW | |
| PSVPDPAHSSLGSWVPTIMEELPGPRQGQWLGQTSEMSRALTPHPCVQDAFQLPG | |
| LGTPPITKLTVLEEDEKKPVPWESHNSSETCGLPTLVQTYVLQGDPRAVSTQPQS | |
| QSGTSDQVLYGQLLGSPTSPGPGHYLRCDSTQPLLAGLTPSPKSYENLWFQASPL | |
| GTLVTPAPSQEDDCVFGPLLNFPLLQGIRVHGMEALGSF | |
| >NP_005364.1 thrombopoietin receptor precursor | 26 |
| [Homo sapiens] | |
| MPSWALFMVTSCLLLAPQNLAQVSSQDVSLLASDSEPLKCFSRTFEDLTCFWDEEE | |
| AAPSGTYQLLYAYPREKPRACPLSSQSMPHFGTRYVCQFPDQEEVRLFFPLHLWVK | |
| NVFLNQTRTQRVLFVDSVGLPAPPSIIKAMGGSQPGELQISWEEPAPEISDFLRYELR | |
| YGPRDPKNSTGPTVIQLIATETCCPALQRPHSASALDQSPCAQPTMPWQDGPKQTSP | |
| SREASALTAEGGSCLISGLQPGNSYWLQLRSEPDGISLGGSWGSWSLPVTVDLPGDA | |
| VALGLQCFTLDLKNVTCQWQQQDHASSQGFFYHSRARCCPRDRYPIWENCEEEEK | |
| TNPGLQTPQFSRCHFKSRNDSIIHILVEVTTAPGTVHSYLGSPFWIHQAVRLPTPNLH | |
| WREISSGHLELEWQHPSSWAAQETCYQLRYTGEGHQDWKVLEPPLGARGGTLELR | |
| PRSRYRLQLRARLNGPTYQGPWSSWSDPTRVETATETAWISLVTALHLVLGLSAVL | |
| GLLLLRWQFPAHYRRLRHALWPSLPDLHRVLGQYLRDTAALSPPKATVSDTCEEVE | |
| PSLLEILPKSSERTPLPLCSSQAQMDYRRLQPSCLGTMPLSVCPPMAESGSCCTTHIA | |
| NHSYLPLSYWQQP | |

In some embodiments, the transmembrane domain of the disclosure is derived from a truncated, or otherwise modified version of the naturally occurring TPOR/MPLR receptor shown in Table 2.

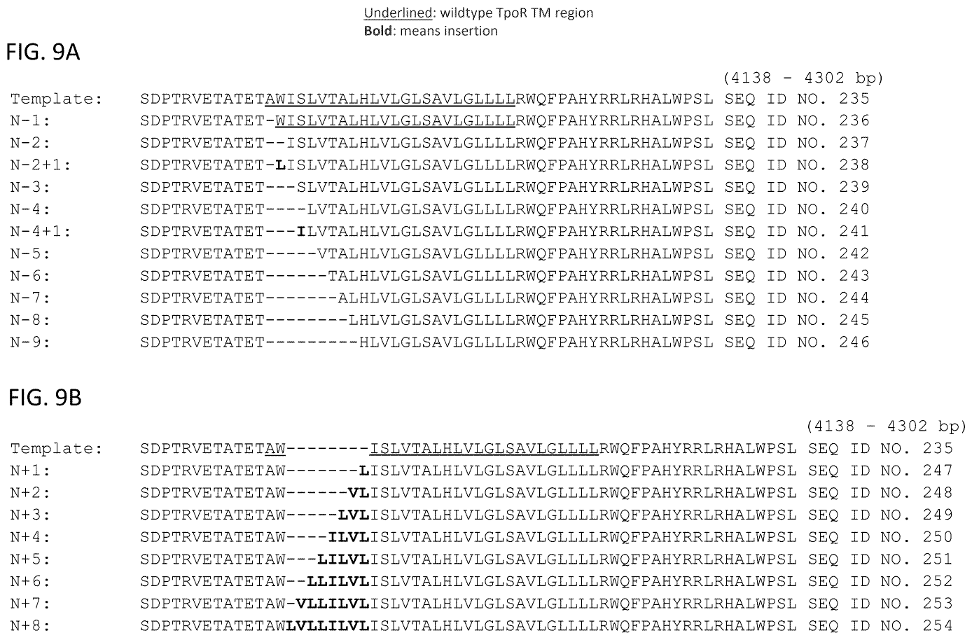

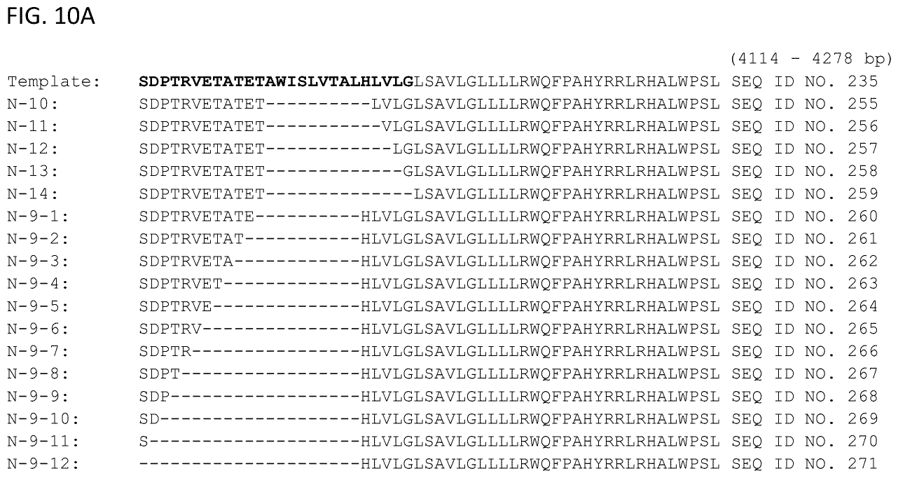

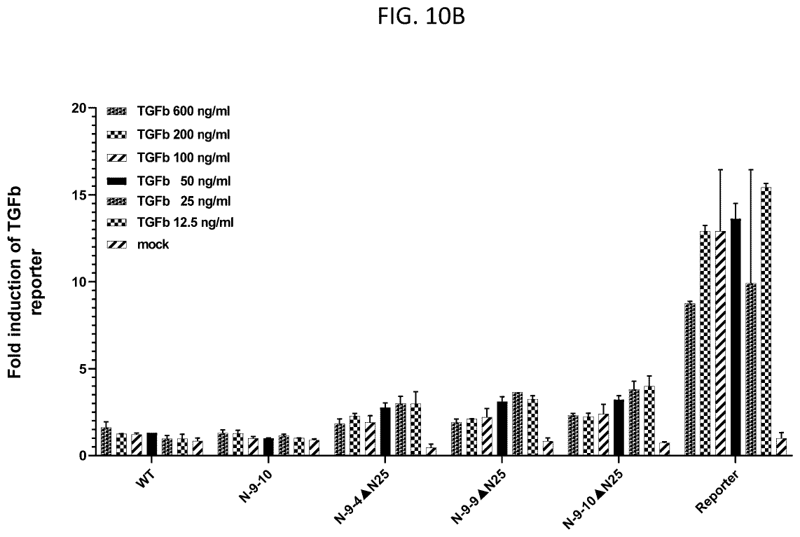

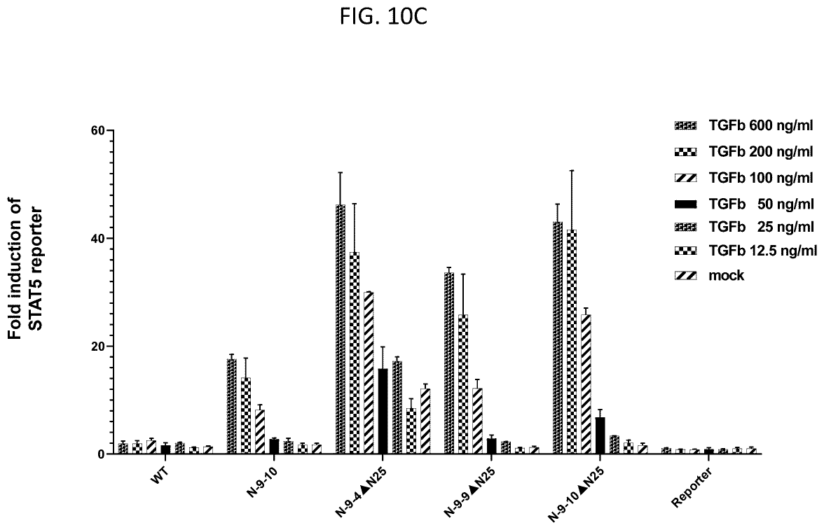

FIGS. 9A-9B and 10A show the amino acid sequences for the wild type TPOR and the various transmembrane deletion (FIGS. 9A, 10A) or insertion (FIG. 9B) variants.

Table 3 shows exemplary transmembrane amino acid sequences, coupled to intracellular JAK2 binding domain sequences.

In some embodiments, the transmembrane domain of the chimeric cytokine receptor comprises the amino acid sequence of SEQ ID NO: 27. In some embodiments, the transmembrane domain of the chimeric cytokine receptor comprises the amino acid sequence of SEQ ID NO: 28. In some embodiments, the transmembrane domain of the chimeric cytokine receptor comprises the amino acid sequence of SEQ ID NO: 29. In some embodiments, the transmembrane domain of the chimeric cytokine receptor comprises the amino acid sequence of SEQ ID NO: 30. In some embodiments, the transmembrane domain of the chimeric cytokine receptor comprises the amino acid sequence of SEQ ID NO: 31. In some embodiments, the transmembrane domain of the chimeric cytokine receptor comprises the amino acid sequence of SEQ ID NO: 32. In some embodiments, the transmembrane domain of the chimeric cytokine receptor comprises the amino acid sequence of SEQ ID NO: 33. In some embodiments, the transmembrane domain of the chimeric cytokine receptor comprises the amino acid sequence of SEQ ID NO: 34. In some embodiments, the transmembrane domain of the chimeric cytokine receptor comprises the amino acid sequence of SEQ ID NO: 35. In some embodiments, the transmembrane domain of the chimeric cytokine receptor comprises the amino acid sequence of SEQ ID NO: 36. In some embodiments, the transmembrane domain of the chimeric cytokine receptor comprises the amino acid sequence of SEQ ID NO: 37. In some embodiments, the transmembrane domain of the chimeric cytokine receptor comprises the amino acid sequence of SEQ ID NO: 38. In some embodiments, the transmembrane domain of the chimeric cytokine receptor comprises the amino acid sequence of SEQ ID NO: 39. In some embodiments, the transmembrane domain of the chimeric cytokine receptor comprises the amino acid sequence of SEQ ID NO: 40. In some embodiments, the transmembrane domain of the chimeric cytokine receptor comprises the amino acid sequence of SEQ ID NO: 41. In some embodiments, the transmembrane domain of the chimeric cytokine receptor comprises the amino acid sequence of SEQ ID NO: 42. In some embodiments, the transmembrane domain of the chimeric cytokine receptor comprises the amino acid sequence of SEQ ID NO: 43. In some embodiments, the transmembrane domain of the chimeric cytokine receptor comprises the amino acid sequence of SEQ ID NO: 44. In some embodiments, the transmembrane domain of the chimeric cytokine receptor comprises the amino acid sequence of SEQ ID NO: 45. In some embodiments, the transmembrane domain of the chimeric cytokine receptor comprises the amino acid sequence of SEQ ID NO: 46. In some embodiments, the transmembrane domain of the chimeric cytokine receptor comprises the amino acid sequence of SEQ ID NO: 47. In some embodiments, the transmembrane domain of the chimeric cytokine receptor comprises the amino acid sequence of SEQ ID NO: 48. In some embodiments, the transmembrane domain of the chimeric cytokine receptor comprises the amino acid sequence of SEQ ID NO: 49. In some embodiments, the transmembrane domain of the chimeric cytokine receptor comprises the amino acid sequence of SEQ ID NO: 50. In some embodiments, the transmembrane domain of the chimeric cytokine receptor comprises the amino acid sequence of SEQ ID NO: 51. In some embodiments, the transmembrane domain of the chimeric cytokine receptor comprises the amino acid sequence of SEQ ID NO: 52. In some embodiments, the transmembrane domain of the chimeric cytokine receptor comprises the amino acid sequence of SEQ ID NO: 53. In some embodiments, the transmembrane domain of the chimeric cytokine receptor comprises the amino acid sequence of SEQ ID NO: 54. In some embodiments, the transmembrane domain of the chimeric cytokine receptor comprises the amino acid sequence of SEQ ID NO: 55. In some embodiments, the transmembrane domain of the chimeric cytokine receptor comprises the amino acid sequence of SEQ ID NO: 56. In some embodiments, the transmembrane domain of the chimeric cytokine receptor comprises the amino acid sequence of SEQ ID NO: 57. In some embodiments, the transmembrane domain of the chimeric cytokine receptor comprises the amino acid sequence of SEQ ID NO: 58. In some embodiments, the transmembrane domain of the chimeric cytokine receptor comprises the amino acid sequence of SEQ ID NO: 59. In some embodiments, the transmembrane domain of the chimeric cytokine receptor comprises the amino acid sequence of SEQ ID NO: 60. In some embodiments, the transmembrane domain of the chimeric cytokine receptor comprises the amino acid sequence of SEQ ID NO: 61. In some embodiments, the transmembrane domain of the chimeric cytokine receptor comprises the amino acid sequence of SEQ ID NO: 62. In some embodiments, the transmembrane domain of the chimeric cytokine receptor comprises the amino acid sequence of SEQ ID NO: 63. In some embodiments, the transmembrane domain of the chimeric cytokine receptor comprises the amino acid sequence of SEQ ID NO: 64. In some embodiments, the transmembrane domain of the chimeric cytokine receptor comprises the amino acid sequence of SEQ ID NO: 65. In some embodiments, the transmembrane domain of the chimeric cytokine receptor comprises the amino acid sequence of SEQ ID NO: 66. In some embodiments, the transmembrane domain of the chimeric cytokine receptor comprises the amino acid sequence of SEQ ID NO: 67. In some embodiments, the transmembrane domain of the chimeric cytokine receptor comprises the amino acid sequence of SEQ ID NO: 68. In some embodiments, the transmembrane domain of the chimeric cytokine receptor comprises the amino acid sequence of SEQ ID NO: 69. In some embodiments, the transmembrane domain of the chimeric cytokine receptor comprises the amino acid sequence of SEQ ID NO: 70. In some embodiments, the transmembrane domain of the chimeric cytokine receptor comprises the amino acid sequence of SEQ ID NO: 71. In some embodiments, the transmembrane domain of the chimeric cytokine receptor comprises the amino acid sequence of SEQ ID NO: 72. In some embodiments, the transmembrane domain of the chimeric cytokine receptor comprises the amino acid sequence of SEQ ID NO: 73. In some embodiments, the transmembrane domain of the chimeric cytokine receptor comprises the amino acid sequence of SEQ ID NO: 74. In some embodiments, the transmembrane domain of the chimeric cytokine receptor comprises the amino acid sequence of SEQ ID NO: 75. In some embodiments, the transmembrane domain of the chimeric cytokine receptor comprises the amino acid sequence of SEQ ID NO: 76. In some embodiments, the transmembrane domain of the chimeric cytokine receptor comprises the amino acid sequence of SEQ ID NO: 77. In some embodiments, the transmembrane domain of the chimeric cytokine receptor comprises the amino acid sequence of SEQ ID NO: 78. In some embodiments, the transmembrane domain of the chimeric cytokine receptor comprises the amino acid sequence of SEQ ID NO: 79. In some embodiments, the transmembrane domain of the chimeric cytokine receptor comprises the amino acid sequence of SEQ ID NO: 160. In some embodiments, the transmembrane domain of the chimeric cytokine receptor comprises the amino acid sequence of SEQ ID NO: 217. In some embodiments, the transmembrane domain of the chimeric cytokine receptor comprises the amino acid sequence of SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, or SEQ ID NO: 234. In some embodiments, the transmembrane domain of the chimeric cytokine receptor comprises an amino acid sequence that is at least 80%, 85%, 90%, 95%, 98%, or 99%, or 100% identical to the amino acid sequence of any one of SEQ ID NOs: 27-79, 160, and 217-234.

In some embodiments, the chimeric cytokine receptor (CCR) comprises the binding domain comprising the amino acid sequence of SEQ ID NO: 3, 4 or 159, and the transmembrane and JAK2 binding domain comprising the amino acid sequence of SEQ ID NO: 64, 69, or 70. In some embodiments, the CCR is inducible. In some embodiments, the CCR comprises the binding domain comprising the amino acid sequence of SEQ ID NO: 3, 4 or 159, and the transmembrane and JAK2 binding domain comprising the amino acid sequence of SEQ ID NO: 38, 39, 40 or 53. In some embodiments, the CCR comprises the binding domain comprising the amino acid sequence of SEQ ID NO: 3, 4 or 159, and the transmembrane and JAK2 binding domain comprising the amino acid sequence of SEQ ID NO: 59, 60, 160, or 217. In some embodiments, the CCR is constitutively active. In some embodiments, the constitutively active CCR of the disclosure dimerizes without a TGF-β ligand.

TABLE 3
Exemplary Transmembrane + JAK2 Binding Domain Sequences

| Transmembrane and JAK2 | | SEQ ID |
|---|---|---|
| binding domain | Amino acid sequence | NO: |
| GCSFR(614-710) | LTLMTLTPEGSELHIILGLFGLLLLLTCLCGTAWLCCSPNRKNPLWP | 27 |
| | SVPDPAHSSLGSWVPTIMEEDAFQLPGLGTPPITKLTVLEEDEKKP | |
| | VPWE | |
| GP130(609-700) | TTPKFAQGEIEAIVVPVCLAFLLTTLLGVLFCFNKRDLIKKHIWPNV | 28 |
| | PDPSKSHIAQWSPHTPPRHNFNSKDQMYSDGNFTDVSWEIEAN | |
| | D | |
| TPOR/MPLR(478-582) | SDPTRVETATETAWISLVTALHLVLGLSAVLGLLLLRWQFPAHYRR | 29 |
| | LRHALWPSLPDLHRVLGQYLRDTAALSPPKATVSDTCEEVEPSLLEI | |
| | LPKSSERTPLPL | |
| TPOR/MPLR(N − 1) | SDPTRVETATETWISLVTALHLVLGLSAVLGLLLLRWQFPAHYRRL | 30 |
| | RHALWPSLPDLHRVLGQYLRDTAALSPPKATVSDTCEEVEPSLLEIL | |
| | PKSSERTPLPL | |
| TPOR/MPLR(N − 2) | SDPTRVETATETISLVTALHLVLGLSAVLGLLLLRWQFPAHYRRLRH | 31 |
| | ALWPSLPDLHRVLGQYLRDTAALSPPKATVSDTCEEVEPSLLEILPK | |
| | SSERTPLPL | |
| TPOR/MPLR(N − 2 + 1) | SDPTRVETATETLISLVTALHLVLGLSAVLGLLLLRWQFPAHYRRLR | 32 |
| | HALWPSLPDLHRVLGQYLRDTAALSPPKATVSDTCEEVEPSLLEILP | |
| | KSSERTPLPL | |
| TPOR/MPLR(N − 3) | SDPTRVETATETSLVTALHLVLGLSAVLGLLLLRWQFPAHYRRLRH | 33 |
| | ALWPSLPDLHRVLGQYLRDTAALSPPKATVSDTCEEVEPSLLEILPK | |
| | SSERTPLPL | |
| TPOR/MPLR(N − 4) | SDPTRVETATETLVTALHLVLGLSAVLGLLLLRWQFPAHYRRLRHA | 34 |
| | LWPSLPDLHRVLGQYLRDTAALSPPKATVSDTCEEVEPSLLEILPKS | |
| | SERTPLPL | |
| TPOR/MPLR(N − 4 + 1) | SDPTRVETATETILVTALHLVLGLSAVLGLLLLRWQFPAHYRRLRHA | 35 |
| | LWPSLPDLHRVLGQYLRDTAALSPPKATVSDTCEEVEPSLLEILPKS | |
| | SERTPLPL | |
| TPOR/MPLR(N − 5) | SDPTRVETATETVTALHLVLGLSAVLGLLLLRWQFPAHYRRLRHAL | 36 |
| | WPSLPDLHRVLGQYLRDTAALSPPKATVSDTCEEVEPSLLEILPKSS | |
| | ERTPLPL | |
| TPOR/MPLR(N − 6) | SDPTRVETATETTALHLVLGLSAVLGLLLLRWQFPAHYRRLRHAL | 37 |
| | WPSLPDLHRVLGQYLRDTAALSPPKATVSDTCEEVEPSLLEILPKSS | |
| | ERTPLPL | |
| TPOR/MPLR(N − 7) | SDPTRVETATETALHLVLGLSAVLGLLLLRWQFPAHYRRLRHALW | 38 |
| | PSLPDLHRVLGQYLRDTAALSPPKATVSDTCEEVEPSLLEILPKSSER | |
| | TPLPL | |
| TPOR/MPLR(N − 8) | SDPTRVETATETLHLVLGLSAVLGLLLLRWQFPAHYRRLRHALWPS | 39 |
| | LPDLHRVLGQYLRDTAALSPPKATVSDTCEEVEPSLLEILPKSSERTP | |
| | LPL | |
| TPOR/MPLR(N − 9) | SDPTRVETATETHLVLGLSAVLGLLLLRWQFPAHYRRLRHALWPSL | 40 |
| | PDLHRVLGQYLRDTAALSPPKATVSDTCEEVEPSLLEILPKSSERTPL | |
| | PL | |
| TPOR/MPLR(N − 10) | SDPTRVETATETLVLGLSAVLGLLLLRWQFPAHYRRLRHALWPSLP | 41 |
| | DLHRVLGQYLRDTAALSPPKATVSDTCEEVEPSLLEILPKSSERTPLP | |
| | L | |
| TPOR/MPLR(N − 11) | SDPTRVETATETVLGLSAVLGLLLLRWQFPAHYRRLRHALWPSLP | 42 |
| | DLHRVLGQYLRDTAALSPPKATVSDTCEEVEPSLLEILPKSSERTPLP | |
| | L | |
| TPOR/MPLR(N − 12) | SDPTRVETATETLGLSAVLGLLLLRWQFPAHYRRLRHALWPSLPDL | 43 |
| | HRVLGQYLRDTAALSPPKATVSDTCEEVEPSLLEILPKSSERTPLPL | |
| TPOR/MPLR(N − 13) | SDPTRVETATETGLSAVLGLLLLRWQFPAHYRRLRHALWPSLPDL | 44 |
| | HRVLGQYLRDTAALSPPKATVSDTCEEVEPSLLEILPKSSERTPLPL | |
| TPOR/MPLR(N − 14) | SDPTRVETATETLSAVLGLLLLRWQFPAHYRRLRHALWPSLPDLH | 45 |
| | RVLGQYLRDTAALSPPKATVSDTCEEVEPSLLEILPKSSERTPLPL | |
| TPOR/MPLR(N − 15) | SDPTRVETATETSAVLGLLLLRWQFPAHYRRLRHALWPSLPDLHR | 46 |
| | VLGQYLRDTAALSPPKATVSDTCEEVEPSLLEILPKSSERTPLPL | |
| TPOR/MPLR(N − 16) | SDPTRVETATETAVLGLLLLRWQFPAHYRRLRHALWPSLPDLHRV | 47 |
| | LGQYLRDTAALSPPKATVSDTCEEVEPSLLEILPKSSERTPLPL | |
| TPOR/MPLR(N − 17) | SDPTRVETATETVLGLLLLRWQFPAHYRRLRHALWPSLPDLHRVL | 48 |
| | GQYLRDTAALSPPKATVSDTCEEVEPSLLEILPKSSERTPLPL | |
| TPOR/MPLR(N − 18) | SDPTRVETATETLGLLLLRWQFPAHYRRLRHALWPSLPDLHRVLG | 49 |
| | QYLRDTAALSPPKATVSDTCEEVEPSLLEILPKSSERTPLPL | |
| TPOR/MPLR(N + 1) | SDPTRVETATETAWLISLVTALHLVLGLSAVLGLLLLRWQFPAHYR | 50 |
| | RLRHALWPSLPDLHRVLGQYLRDTAALSPPKATVSDTCEEVEPSLL | |
| | EILPKSSERTPLPL | |
| TPOR/MPLR(N + 2) | SDPTRVETATETAWVLISLVTALHLVLGLSAVLGLLLLRWQFPAHY | 51 |
| | RRLRHALWPSLPDLHRVLGQYLRDTAALSPPKATVSDTCEEVEPSL | |
| | LEILPKSSERTPLPL | |
| TPOR/MPLR(N + 3) | SDPTRVETATETAWLVLISLVTALHLVLGLSAVLGLLLLRWQFPAH | 52 |
| | YRRLRHALWPSLPDLHRVLGQYLRDTAALSPPKATVSDTCEEVEPS | |
| | LLEILPKSSERTPLPL | |
| TPOR/MPLR(N + 4) | SDPTRVETATETAWILVLISLVTALHLVLGLSAVLGLLLLRWQFPAH | 53 |
| | YRRLRHALWPSLPDLHRVLGQYLRDTAALSPPKATVSDTCEEVEPS | |
| | LLEILPKSSERTPLPL | |
| TPOR/MPLR(N + 5) | SDPTRVETATETAWLILVLISLVTALHLVLGLSAVLGLLLLRWQFPA | 54 |
| | HYRRLRHALWPSLPDLHRVLGQYLRDTAALSPPKATVSDTCEEVE | |
| | PSLLEILPKSSERTPLPL | |
| TPOR/MPLR(N + 6) | SDPTRVETATETAWLLILVLISLVTALHLVLGLSAVLGLLLLRWQFP | 55 |
| | AHYRRLRHALWPSLPDLHRVLGQYLRDTAALSPPKATVSDTCEEV | |
| | EPSLLEILPKSSERTPLPL | |
| TPOR/MPLR(N + 7) | SDPTRVETATETAWVLLILVLISLVTALHLVLGLSAVLGLLLLRWQF | 56 |
| | PAHYRRLRHALWPSLPDLHRVLGQYLRDTAALSPPKATVSDTCEE | |
| | VEPSLLEILPKSSERTPLPL | |
| TPOR/MPLR(N + 8) | SDPTRVETATETAWLVLLILVLISLVTALHLVLGLSAVLGLLLLRWQF | 57 |
| | PAHYRRLRHALWPSLPDLHRVLGQYLRDTAALSPPKATVSDTCEE | |
| | VEPSLLEILPKSSERTPLPL | |
| TPOR/MPLR(478- | SDPTRVETATETAWISLVTALHLVLGLSAVLGLLLLRKQFPAHYRRL | 58 |
| 582; W515K) | RHALWPSLPDLHRVLGQYLRDTAALSPPKATVSDTCEEVEPSLLEIL | |
| | PKSSERTPLPL | |
| TPOR/MPLR(478- | SDPTRVETATETAWISLVTALLLVLGLNAVLGLLLLRKQFPAHYRRL | 59 |
| 582; H499L, | RHALWPSLPDLHRVLGQYLRDTAALSPPKATVSDTCEEVEPSLLEIL | |
| S505N, W515K) | PKSSERTPLPL | |
| TPOR/MPLR(478- | SDPTRVETATETAWISLVTALHLVLGLNAVLGLLLLRKQFPAHYRR | 60 |
| 582; S505N, W515K) | LRHALWPSLPDLHRVLGQYLRDTAALSPPKATVSDTCEEVEPSLLEI | |
| | LPKSSERTPLPL | |
| TPOR/MPLR(N − 9 − 1) | SDPTRVETATEHLVLGLSAVLGLLLLRWQFPAHYRRLRHALWPSL | 61 |
| | PDLHRVLGQYLRDTAALSPPKATVSDTCEEVEPSLLEILPKSSERTPL | |
| | PL | |
| TPOR/MPLR(N − 9 − 2) | SDPTRVETATHLVLGLSAVLGLLLLRWQFPAHYRRLRHALWPSLP | 62 |
| | DLHRVLGQYLRDTAALSPPKATVSDTCEEVEPSLLEILPKSSERTPLP | |
| | L | |
| TPOR/MPLR(N − 9 − 3) | SDPTRVETAHLVLGLSAVLGLLLLRWQFPAHYRRLRHALWPSLPD | 63 |
| | LHRVLGQYLRDTAALSPPKATVSDTCEEVEPSLLEILPKSSERTPLPL | |
| TPOR/MPLR(N − 9 − 4) | SDPTRVETHLVLGLSAVLGLLLLRWQFPAHYRRLRHALWPSLPDL | 64 |
| | HRVLGQYLRDTAALSPPKATVSDTCEEVEPSLLEILPKSSERTPLPL | |
| TPOR/MPLR(N − 9 − 5) | SDPTRVEHLVLGLSAVLGLLLLRWQFPAHYRRLRHALWPSLPDLH | 65 |
| | RVLGQYLRDTAALSPPKATVSDTCEEVEPSLLEILPKSSERTPLPL | |
| TPOR/MPLR(N − 9 − G) | SDPTRVHLVLGLSAVLGLLLLRWQFPAHYRRLRHALWPSLPDLHR | 66 |
| | VLGQYLRDTAALSPPKATVSDTCEEVEPSLLEILPKSSERTPLPL | |
| TPOR/MPLR(N − 9 − 7) | SDPTRHLVLGLSAVLGLLLLRWQFPAHYRRLRHALWPSLPDLHRV | 67 |
| | LGQYLRDTAALSPPKATVSDTCEEVEPSLLEILPKSSERTPLPL | |
| TPOR/MPLR(N − 9 − 8) | SDPTHLVLGLSAVLGLLLLRWQFPAHYRRLRHALWPSLPDLHRVL | 68 |
| | GQYLRDTAALSPPKATVSDTCEEVEPSLLEILPKSSERTPLPL | |
| TPOR/MPLR(N − 9 − 9) | SDPHLVLGLSAVLGLLLLRWQFPAHYRRLRHALWPSLPDLHRVLG | 69 |
| | QYLRDTAALSPPKATVSDTCEEVEPSLLEILPKSSERTPLPL | |
| TPOR/MPLR(N − 9 − 10) | SDHLVLGLSAVLGLLLLRWQFPAHYRRLRHALWPSLPDLHRVLGQ | 70 |
| | YLRDTAALSPPKATVSDTCEEVEPSLLEILPKSSERTPLPL | |
| TPOR/MPLR(N − 9 − 11) | SHLVLGLSAVLGLLLLRWQFPAHYRRLRHALWPSLPDLHRVLGQY | 71 |
| | LRDTAALSPPKATVSDTCEEVEPSLLEILPKSSERTPLPL | |
| TPOR/MPLR(N − 9 − 12) | HLVLGLSAVLGLLLLRWQFPAHYRRLRHALWPSLPDLHRVLGQYL | 72 |
| | RDTAALSPPKATVSDTCEEVEPSLLEILPKSSERTPLPL | |
| TPOR/MPLR(N − C3) | SDPTRVETATETAWISLVHLVLGLSAVLGLLLLRWQFPAHYRRLRH | 73 |
| | ALWPSLPDLHRVLGQYLRDTAALSPPKATVSDTCEEVEPSLLEILPK | |
| | SSERTPLPL | |
| TPOR/MPLR(N − C5) | SDPTRVETATETAWISHLVLGLSAVLGLLLLRWQFPAHYRRLRHAL | 74 |
| | WPSLPDLHRVLGQYLRDTAALSPPKATVSDTCEEVEPSLLEILPKSS | |
| | ERTPLPL | |
| TPOR/MPLR(N − C5P) | SDPTRVETATETAWISPHLVLGLSAVLGLLLLRWQFPAHYRRLRHA | 75 |
| | LWPSLPDLHRVLGQYLRDTAALSPPKATVSDTCEEVEPSLLEILPKS | |
| | SERTPLPL | |
| TPOR/MPLR(N − C5PP) | SDPTRVETATETAWISPPHLVLGLSAVLGLLLLRWQFPAHYRRLRH | 76 |
| | ALWPSLPDLHRVLGQYLRDTAALSPPKATVSDTCEEVEPSLLEILPK | |
| | SSERTPLPL | |
| TPOR/MPLR(N − C6) | SDPTRVETATETAWIHLVLGLSAVLGLLLLRWQFPAHYRRLRHAL | 77 |
| | WPSLPDLHRVLGQYLRDTAALSPPKATVSDTCEEVEPSLLEILPKSS | |
| | ERTPLPL | |
| TPOR/MPLR(N − C6P) | SDPTRVETATETAWIPHLVLGLSAVLGLLLLRWQFPAHYRRLRHAL | 78 |
| | WPSLPDLHRVLGQYLRDTAALSPPKATVSDTCEEVEPSLLEILPKSS | |
| | ERTPLPL | |
| TPOR/MPLR(N − C6PP) | SDPTRVETATETAWIPPHLVLGLSAVLGLLLLRWQFPAHYRRLRH | 79 |
| | ALWPSLPDLHRVLGQYLRDTAALSPPKATVSDTCEEVEPSLLEILPK | |
| | SSERTPLPL | |
| TPOR/MPLR(478-582; | SDPTRVETATETAWISLVTALLLVLGLNAVLGLLLLRKQFPAHYRRL | 217 |
| H499L, S505N, | RHALWPSLPDLHRVLGQYLRDTAALSPPRATVSDTCEEVEPSLLEIL | |
| W515K, K553R, | PRSSERTPLPL | |
| K573R) | | |
| TPOR/MPLR(478-582) | SDPTRVETATETAWISLVTALHLVLGLNAVLGLLLLRKQFPAHYRR | 160 |
| S505N.W515K. | LRHALWPSLPDLHRVLGQYLRDTAALSPPRATVSDTCEEVEPSLLE | |
| K553R.K573R | ILPRSSERTPLPL | |
| TPOR/MPLR(478-582) | SDPTRVETATETAWISLVTALHLVLGLSAVLGLLLLRWQFPAHYRR | 218 |
| K553R.K573R | LRHALWPSLPDLHRVLGQYLRDTAALSPPRATVSDTCEEVEPSLLE | |
| | ILPRSSERTPLPL | |
| TPOR/MPLR(N − 7) | SDPTRVETATETALHLVLGLNAVLGLLLLRKQFPAHYRRLRHALWP | 219 |
| S505N.W515K | SLPDLHRVLGQYLRDTAALSPPKATVSDTCEEVEPSLLEILPKSSERT | |
| | PLPL | |
| TPOR/MPLR(N − 7) | SDPTRVETATETALHLVLGLNAVLGLLLLRKQFPAHYRRLRHALWP | 220 |
| S505N.W515K. | SLPDLHRVLGQYLRDTAALSPPRATVSDTCEEVEPSLLEILPRSSERT | |
| K553R, K573R | PLPL | |
| TPOR/MPLR(N − 8) | SDPTRVETATETLHLVLGLNAVLGLLLLRKQFPAHYRRLRHALWPS | 221 |
| S505N.W515K | LPDLHRVLGQYLRDTAALSPPKATVSDTCEEVEPSLLEILPKSSERTP | |
| | LPL | |
| TPOR/MPLR(N − 8) | SDPTRVETATETLHLVLGLNAVLGLLLLRKQFPAHYRRLRHALWPS | 222 |
| S505N.W515K. | LPDLHRVLGQYLRDTAALSPPRATVSDTCEEVEPSLLEILPRSSERT | |
| K553R, K573R | PLPL | |
| TPOR/MPLR(N − 9) | SDPTRVETATETHLVLGLNAVLGLLLLRKQFPAHYRRLRHALWPSL | 223 |
| S505N.W515K | PDLHRVLGQYLRDTAALSPPKATVSDTCEEVEPSLLEILPKSSERTPL | |
| | PL | |
| TPOR/MPLR(N − 9) | SDPTRVETATETHLVLGLNAVLGLLLLRKQFPAHYRRLRHALWPSL | 224 |
| S505N.W515K. | PDLHRVLGQYLRDTAALSPPRATVSDTCEEVEPSLLEILPRSSERTP | |
| K553R, BK573R | LPL | |
| TPOR/MPLR(N + 4) | SDPTRVETATETAWILVLISLVTALHLVLGLNAVLGLLLLRKQFPAH | 225 |
| S505N.W515K | YRRLRHALWPSLPDLHRVLGQYLRDTAALSPPKATVSDTCEEVEPS | |
| | LLEILPKSSERTPLPL | |
| TPOR/MPLR(N + 4) | SDPTRVETATETAWILVLISLVTALHLVLGLNAVLGLLLLRKQFPAH | 226 |
| S505N.W515K. | YRRLRHALWPSLPDLHRVLGQYLRDTAALSPPRATVSDTCEEVEPS | |
| K553R, K573R | LLEILPRSSERTPLPL | |
| TPOR/M PLR(N − 9 − 1) | SDPTRVETATEHLVLGLNAVLGLLLLRKQFPAHYRRLRHALWPSLP | 227 |
| S505N.W515K | DLHRVLGQYLRDTAALSPPKATVSDTCEEVEPSLLEILPKSSERTPLP | |
| | L | |
| TPOR/MPLR(N − 9 − 1) | SDPTRVETATEHLVLGLNAVLGLLLLRKQFPAHYRRLRHALWPSLP | 228 |
| S505N.W515K. | DLHRVLGQYLRDTAALSPPRATVSDTCEEVEPSLLEILPRSSERTPL | |
| K553R, K573R | PL | |
| TPOR/M PLR(N − 9 − 4) | SDPTRVETHLVLGLNAVLGLLLLRKQFPAHYRRLRHALWPSLPDLH | 229 |
| S505N.W515K | RVLGQYLRDTAALSPPKATVSDTCEEVEPSLLEILPKSSERTPLPL | |
| TPOR/MPLR(N − 9 − 4) | SDPTRVETHLVLGLNAVLGLLLLRKQFPAHYRRLRHALWPSLPDLH | 230 |
| S505N.W515K. | RVLGQYLRDTAALSPPRATVSDTCEEVEPSLLEILPRSSERTPLPL | |
| K553R, K573R | | |
| TPOR/M PLR(N − 9 − 9) | SDPHLVLGLNAVLGLLLLRKQFPAHYRRLRHALWPSLPDLHRVLG | 231 |
| S505N.W515K | QYLRDTAALSPPKATVSDTCEEVEPSLLEILPKSSERTPLPL | |
| TPOR/MPLR(N − 9 − 9) | SDPHLVLGLNAVLGLLLLRKQFPAHYRRLRHALWPSLPDLHRVLG | 232 |
| S505N.W515K. | QYLRDTAALSPPRATVSDTCEEVEPSLLEILPRSSERTPLPL | |
| K553R, K573R | | |
| TPOR/MPLR(N − 9 − 10) | SDHLVLGLNAVLGLLLLRKQFPAHYRRLRHALWPSLPDLHRVLGQ | 233 |
| S505N.W515K | YLRDTAALSPPKATVSDTCEEVEPSLLEILPKSSERTPLPL | |
| TPOR/MPLR(N − 9 − 10) | SDHLVLGLNAVLGLLLLRKQFPAHYRRLRHALWPSLPDLHRVLGQ | 234 |
| S505N.W515K. | YLRDTAALSPPRATVSDTCEEVEPSLLEILPRSSERTPLPL | |
| K553R, K573R | | |

### C. Janus Kinase (JAK)-Binding Domains

The chimeric cytokine receptors of the disclosure comprise intracellular JAK-binding domains. The JAK-binding domain is coupled to the C-terminus of the transmembrane domain, either directly, or via a linker. The JAK-binding domain is coupled to the transmembrane domain on the intracellular side of the chimeric cytokine receptor.

In some embodiments, the JAK-binding domain is a JAK-1-binding domain, a JAK-2 binding domain, a JAK-3 binding domain, or a TYK2 binding domain.

In some embodiments, the JAK-binding domains of the chimeric cytokine receptors of the disclosure are naturally occurring, and derived from a naturally occurring receptor.

In some embodiments, the JAK-binding domains of the chimeric cytokine receptors of the disclosure are synthetic.

In some embodiments, the chimeric cytokine receptor comprises a transmembrane and JAK2 binding domain that is at least 80%, 85%, 90%, 95%, 98% or 99%, or 100% identical to any one of the amino acid sequences of SEQ ID NOs: 27-79, 160 and 217-234.

Table 3 provides exemplary amino acid sequences for the transmembrane and JAK2 binding domains of the disclosure. In some embodiments, the transmembrane and JAK2 binding domain comprises one or more mutations, e.g., one or more deletions, insertions and/or substitutions of the wild type sequences. In some embodiments, the transmembrane and JAK2 binding domain comprises one or more substitutions at amino acid positions H499, S505 and W515 of the wild type TPOR/MPLR sequence. See Table 3. In some embodiments, the transmembrane and JAK2 binding domain comprises one or more substitutions at the amino acid positions K533 and K573 of the wild type TPOR/MPLR sequence. In some embodiments, the transmembrane and JAK2 binding domain, e.g., as shown in Table 3, may be combined with a TGFβR2 ectodomain as disclosed herein, e.g., in Table 1, or a PD-1 ectodomain (such as a high affinity PD-1 ectodomain, as indicated in SEQ ID NO: 274 or 275 in Table 6) and a recruiting domain to form a chimeric cytokine receptor. In some embodiments, the transmembrane and JAK2 binding domain may be combined with a recruiting domain to form a chimeric cytokine receptor without an ectodomain, see e.g., SEQ ID NOs: 272 or 273. See also U.S. Ser. No. 16/804,917, filed on Feb. 28, 2020, and U.S. Ser. No. 16/804,545, filed on Feb. 28, 2020, both of which are incorporated herein by reference in their entireties.

### D. Recruiting Domains

The chimeric cytokine receptors of the disclosure comprise cytoplasmic domains comprising recruiting domains (which may also be referred to as “signaling domains”). The recruiting domain can be a STAT-recruiting domain, an AP1—recruiting domain, a Myc/Max recruiting domain; or an NFkB-recruiting domain. In some embodiments, the recruiting domain is a Signal Transducer and Activator of Transcription (STAT)—recruiting (Stat-activating) domains from receptor tails (cytotails) or from cytokine receptor tails. These intracellular recruiting domains of the chimeric cytokine receptors of the disclosure allow for the propagation of Signal 3 in an immune cell comprising a CAR and a chimeric cytokine receptor (e.g. a CAR-T-cell with a chimeric cytokine receptor of the disclosure). Cytokine signaling propagated through the Stat-recruiting domain allows for the cytokine-based immune potentiation of the cell. In some embodiments, the immune-potentiation is homeostatic, e.g. signaling gives rise to increase in immune cells bearing the CAR. In some embodiments, the immune-potentiation is inflammatory, e.g. signaling gives rise to increase in the potency of the immune cells bearing the CAR. In some embodiments, the immune-potentiation prevents exhaustion, e.g. signaling maintains the long-term functionality of immune cells bearing the CAR.

In some embodiments, the recruiting domains of the disclosure are synthetic, and do not resemble any naturally occurring receptor fragment.

In some embodiments, the Stat-recruiting domains of the disclosure are synthetic, and do not resemble any naturally occurring receptor fragment.

In other embodiments, the Stat-recruiting domains of the disclosure are derived from cytoplasmic tails of naturally occurring receptors, e.g. derived from naturally occurring cytokine receptors. In some embodiments, the chimeric cytokine receptor comprises a portion or a fragment of a naturally occurring receptor, e.g., the intracellular Stat-recruiting domain of the naturally occurring receptor, optionally with one or more mutations therein (e.g., one or more deletions, insertions and/or substitutions). These cytoplasmic tails of naturally occurring receptors may be the regions downstream of the JAK-activating domains of the transmembrane domain of the receptor. The Stat-recruiting domains of the chimeric cytokine receptors comprise at least one STAT-recruiting domain from at least one receptor. In some embodiments, the Stat-recruiting domain comprises at least one STAT1-recruiting domain. In some embodiments, the Stat-recruiting domain comprises at least one STAT2-recruiting domain. In some embodiments, the Stat-recruiting domain comprises at least one STAT3-recruiting domain. In some embodiments, the Stat-recruiting domain comprises at least one STAT4-recruiting domain. In some embodiments, the Stat-recruiting domain comprises at least one STAT5-recruiting domain. In some embodiments, the STAT-recruiting domain comprises at least one STAT6-recruiting domain. In some embodiments, the Stat-recruiting domain comprises at least one STAT7-recruiting domain.

In some embodiments, the naturally occurring receptor from which the STAT-recruiting domain is derived, is a not a cytokine receptor.

In some embodiments, the naturally occurring receptor from which the Stat-recruiting domain is derived, is a cytokine receptor. Exemplary cytokine receptors through which T-cell-immune potentiating cytokines signal include, but are not limited to IL-2 receptor, IL-7 receptor, IL-15 receptor, IL12 receptor, and IL-21 receptor. In some embodiments, the cytokine receptor from which the STAT-recruiting domain is derived contains phosphorylatable tyrosine residues downstream of the cognate JAK-binding motifs, and one or more signaling domains of interest may be fused downstream of the transmembrane domain to generate single or multiple signaling outputs. In alternative embodiments, the receptor from which the Stat-recruiting domain is derived, is not a cytokine receptor. By choosing the Stat-recruiting domain of the chimeric cytokine receptor, the receptor can be redirected to signaling of choice. In some embodiments, the chimeric cytokine receptor comprises two or more Stat-recruiting domains from more than one receptor. In some embodiments, the two or more Stat-recruiting domains are linked with or without a peptide linker.

Table 4 provides exemplary receptors from which Stat-recruiting domains (signaling domains) of the chimeric cytokine receptors of the disclosure are derived. Table 5a provides exemplary amino acid sequences of recruiting domains of the disclosure.

In some embodiments, the chimeric cytokine receptor of the disclosure comprises the amino acid sequence of the STAT-recruiting domain of SEQ ID NO: 80. In some embodiments, the chimeric cytokine receptor of the disclosure comprises the amino acid sequence of the STAT-recruiting domain of SEQ ID NO: 81. In some embodiments, the chimeric cytokine receptor of the disclosure comprises the amino acid sequence of the STAT-recruiting domain of SEQ ID NO: 82. In some embodiments, the chimeric cytokine receptor of the disclosure comprises the amino acid sequence of the STAT-recruiting domain of SEQ ID NO: 83. In some embodiments, the chimeric cytokine receptor of the disclosure comprises the amino acid sequence of the STAT-recruiting domain of SEQ ID NO: 84. In some embodiments, the chimeric cytokine receptor of the disclosure comprises the amino acid sequence of the STAT-recruiting domain of SEQ ID NO: 85. In some embodiments, the chimeric cytokine receptor of the disclosure comprises the amino acid sequence of the STAT-recruiting domain of SEQ ID NO: 86. In some embodiments, the chimeric cytokine receptor of the disclosure comprises the amino acid sequence of the STAT-recruiting domain of SEQ ID NO: 87. In some embodiments, the chimeric cytokine receptor of the disclosure comprises the amino acid sequence of the STAT-recruiting domain of SEQ ID NO: 88. In some embodiments, the chimeric cytokine receptor of the disclosure comprises the amino acid sequence of the STAT-recruiting domain of SEQ ID NO: 89. In some embodiments, the chimeric cytokine receptor of the disclosure comprises the amino acid sequence of the STAT-recruiting domain of SEQ ID NO: 90. In some embodiments, the chimeric cytokine receptor of the disclosure comprises the amino acid sequence of the STAT-recruiting domain of SEQ ID NO: 91. In some embodiments, the chimeric cytokine receptor of the disclosure comprises the amino acid sequence of the STAT-recruiting domain of SEQ ID NO: 92. In some embodiments, the chimeric cytokine receptor of the disclosure comprises the amino acid sequence of the STAT-recruiting domain of SEQ ID NO: 93. In some embodiments, the chimeric cytokine receptor of the disclosure comprises the amino acid sequence of the STAT-recruiting domain of SEQ ID NO: 94. In some embodiments, the chimeric cytokine receptor of the disclosure comprises the amino acid sequence of the STAT-recruiting domain of SEQ ID NO: 95. In some embodiments, the chimeric cytokine receptor of the disclosure comprises the amino acid sequence of the STAT-recruiting domain of SEQ ID NO: 96. In some embodiments, the chimeric cytokine receptor of the disclosure comprises the amino acid sequence of the STAT-recruiting domain of SEQ ID NO: 97. In some embodiments, the chimeric cytokine receptor of the disclosure comprises the amino acid sequence of the STAT-recruiting domain of SEQ ID NO: 98. In some embodiments, the chimeric cytokine receptor of the disclosure comprises the amino acid sequence of the STAT-recruiting domain of SEQ ID NO: 99. In some embodiments, the chimeric cytokine receptor of the disclosure comprises the amino acid sequence of the STAT-recruiting domain of SEQ ID NO: 100. In some embodiments, the chimeric cytokine receptor of the disclosure comprises the amino acid sequence of the STAT-recruiting domain of SEQ ID NO: 101. In some embodiments, the chimeric cytokine receptor of the disclosure comprises the amino acid sequence of the STAT-recruiting domain of SEQ ID NO: 102. In some embodiments, the chimeric cytokine receptor of the disclosure comprises the amino acid sequence of the STAT-recruiting domain of SEQ ID NO: 103. In some embodiments, the chimeric cytokine receptor of the disclosure comprises the amino acid sequence of the STAT-recruiting domain of SEQ ID NO: 104. In some embodiments, the chimeric cytokine receptor of the disclosure comprises the amino acid sequence of the STAT-recruiting domain of SEQ ID NO: 105. In some embodiments, the chimeric cytokine receptor of the disclosure comprises the amino acid sequence of the STAT-recruiting domain of SEQ ID NO: 106. In some embodiments, the chimeric cytokine receptor of the disclosure comprises the amino acid sequence of the STAT-recruiting domain of SEQ ID NO: 107. In some embodiments, the chimeric cytokine receptor of the disclosure comprises the amino acid sequence of the STAT-recruiting domain of SEQ ID NO: 108. In some embodiments, the chimeric cytokine receptor of the disclosure comprises the amino acid sequence of the STAT-recruiting domain of SEQ ID NO: 109. In some embodiments, the chimeric cytokine receptor of the disclosure comprises the amino acid sequence of the STAT-recruiting domain of SEQ ID NO: 110. In some embodiments, the chimeric cytokine receptor of the disclosure comprises the amino acid sequence of the STAT-recruiting domain of SEQ ID NO: 111. In some embodiments, the chimeric cytokine receptor of the disclosure comprises the amino acid sequence of the STAT-recruiting domain of SEQ ID NO: 112. In some embodiments, the chimeric cytokine receptor of the disclosure comprises the amino acid sequence of the STAT-recruiting domain of SEQ ID NO: 113. In some embodiments, the chimeric cytokine receptor of the disclosure comprises the amino acid sequence of the STAT-recruiting domain of SEQ ID NO: 114. In some embodiments, the chimeric cytokine receptor of the disclosure comprises the amino acid sequence of the STAT-recruiting domain of SEQ ID NO: 115. In some embodiments, the chimeric cytokine receptor of the disclosure comprises the amino acid sequence of the STAT-recruiting domain of SEQ ID NO: 116. In some embodiments, the chimeric cytokine receptor of the disclosure comprises the amino acid sequence of the STAT-recruiting domain of SEQ ID NO: 117. In some embodiments, the chimeric cytokine receptor of the disclosure comprises the amino acid sequence of the STAT-recruiting domain of SEQ ID NO: 118. In some embodiments, the chimeric cytokine receptor of the disclosure comprises the amino acid sequence of the STAT-recruiting domain of SEQ ID NO: 119. In some embodiments, the chimeric cytokine receptor of the disclosure comprises the amino acid sequence of the STAT-recruiting domain of SEQ ID NO: 120. In some embodiments, the chimeric cytokine receptor of the disclosure comprises the amino acid sequence of the STAT-recruiting domain of SEQ ID NO: 121. In some embodiments, the chimeric cytokine receptor of the disclosure comprises the amino acid sequence of the STAT-recruiting domain of SEQ ID NO: 122. In some embodiments, the chimeric cytokine receptor of the disclosure comprises the amino acid sequence of the STAT-recruiting domain of SEQ ID NO: 161. In some embodiments, the chimeric cytokine receptor comprises a recruiting domain that comprises an amino acid sequence that is at least 80%, 85%, 90%, 95%, 98%, or 99%, or 100% identical to any one of the amino acid sequences of SEQ ID NOs: 80-122 and SEQ ID NO: 161.

TABLE 4
Recruiting domain sources
Source for recruiting domains

BLNK
IL2RG
EGFR
EpoR
GHR
IFNAR1
IFNAR2
IFNAR1/2
IFNLR1
IL10R1
IL12Rb1
IL12Rb2
IL21R
IL2Rb
IL2small
IL7R
IL7Ra
IL9R
IL15R
IL21R

TABLE 5a
Recruiting Domain Sequences (Cytotail Sequences)

| | | SEQ ID |
|---|---|---|
| Recruiting domain | Amino acid sequence | NO: |
| IL7R(316-459) | ARDEVEGFLQDTFPQQLEESEKQRLGGDVQSPNCPSEDVVITPES | 80 |
| | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQ | |
| IL2Rb(333-551) | VTQLLLQQDKVPEPASLSSNHSLTSCFTNQGYFFFHLPDALEIEAC | 81 |
| | QVYFTYDPYSEEDPDEGVAGAPTGSSPQPLQPLSGEDDAYCTFPS | |
| | RDDLLLFSPSLLGGPSPPSTAPGGSGAGEERMPPSLQERVPRDW | |
| | DPQPLGPPTPGVPDLVDFQPPPELVLREAGEEVPDAGPREGVSFP | |
| | WSRPPGQGEFRALNARLPLNTDAYLSLQELQGQDPTHLV | |
| IFNAR1(508-557) | ISTIATVEETNQTDEDHKKYSSQTSQDSGNYSNEDESESKTSEELQ | 82 |
| | QDFV | |
| IFNAR2(310-515) | KKKVWDYNYDDESDSDTEAAPRTSGGGYTMHGLTVRPLGQASA | 83 |
| | TSTESQLIDPESEEEPDLPEVDVELPTMPKDSPQQLELLSGPCERRK | |
| | SPLQDPFPEEDYSSTEGSGGRITFNVDLNSVFLRVLDDEDSDDLEA | |
| | PLMLSSHLEEMVDPEDPDNVQSNHLLASGEGTQPTFPSPSSEGL | |
| | WSEDAPSDQSDTSESDVDLGDGYIMR | |
| IFNAR1/2(IFNAR1 residues | ISTIATVEETNQTDEDHKKYSSQTSQDSGNYSNEDESESKTSEELQ | 84 |
| 508-557-IFNAR2 residues | QDFVKKKVWDYNYDDESDSDTEAAPRTSGGGYTMHGLTVRPLG | |
| 310-515) | QASATSTESQLIDPESEEEPDLPEVDVELPTMPKDSPQQLELLSGP | |
| | CERRKSPLQDPFPEEDYSSTEGSGGRITFNVDLNSVFLRVLDDEDS | |
| | DDLEAPLMLSSHLEEMVDPEDPDNVQSNHLLASGEGTQPTFPSP | |
| | SSEGLWSEDAPSDQSDTSESDVDLGDGYIMR | |
| IFNLR1(300-520) | RGVRPTPRVRAPATQQTRWKKDLAEDEEEEDEEDTEDGVSFQPY | 85 |
| | IEPPSFLGQEHQAPGHSEAGGVDSGRPRAPLVPSEGSSAWDSSD | |
| | RSWASTVDSSWDRAGSSGYLAEKGPGQGPGGDGHQESLPPPEF | |
| | SKDSGFLEELPEDNLSSWATWGTLPPEPNLVPGGPPVSLQTLTFC | |
| | WESSPEEEEEARESEIEDSDAGSWGAESTQRTEDRGRTLGHYMA | |
| | R | |
| Common Gamma | IPPKGGALGEGPGASPCNQHSPYWAPPCYTLKPET | 86 |
| Chain (335-369) | | |
| IL9R(356-521) | TALLTCGPARPWKSVALEEEQEGPGTRLPGNLSSEDVLPAGCTEW | 87 |
| | RVQTLAYLPQEDWAPTSLTRPAPPDSEGSRSSSSSSSSNNNNYCA | |
| | LGCYGGWHLSALPGNTQSSGPIPALACGLSCDHQGLETQQGVA | |
| | WVLAGHCQRPGLHEDLQGMLLPSVLSKARSWTF | |
| IL21R(322-538) | PRSPAKRLQLTELQEPAELVESDGVPKPSFWPTAQNSGGSAYSEE | 88 |
| | RDRPYGLVSIDTVTVLDAEGPCTWPCSCEDDGYPALDLDAGLEPS | |
| | PGLEDPLLDAGTTVLSCGCVSAGSPGLGGPLGSLLDRLKPPLADGE | |
| | DWAGGLPWGGRSPGGVSESEAGSPLAGLDMDTFDSGFVGSDCS | |
| | SPVECDFTSPGDEGPPRSYLRQWVVIPPPLSSPGPQAS | |
| GHR(353-638) | PDEKTEESDTDRLLSSDHEKSHSNLGVKDGDSGRTSCCEPDILETD | 89 |
| | FNANDIHEGTSEVAQPQRLKGEADLLCLDQKNQNNSPYHDACPA | |
| | TQQPSVIQAEKNKPQPLPTEGAESTHQAAHIQLSNPSSLSNIDFYA | |
| | QVSDITPAGSVVLSPGQKNKAGMSQCDMHPEMVSLCQENFLM | |
| | DNAYFCEADAKKCIPVAPHIKVESHIQPSLNQEDIYITTESLTTAAG | |
| | RPGTGEHVPGSEMPVPDYTSIHIVQSPQGLILNATALPLPDKEFLS | |
| | SCGYVSTDQLNKIMP | |
| EpoR(339-508) | WGTMQAVEPGTDDEGPLLEPVGSEHAQDTYLVLDKWLLPRNPP | 90 |
| | SEDLPGPGGSVDIVAMDEGSEASSCSSALASKPSPEGASAASFEYT | |
| | ILDPSSQLLRPWTLCPELPPTPPHLKYLYLVVSDSGISTDYSSGDSQ | |
| | GAQGGLSDGPYSNPYENSLIPAAEPLPPSYVACS | |
| murine IL2Rb(337-539) | AVQLLLLQKDSAPLPSPSGHSQASCFTNQGYFFFHLPNALEIESCQ | 91 |
| | VYFTYDPCVEEEVEEDGSRLPEGSPHPPLLPLAGEQDDYCAFPPRD | |
| | DLLLFSPSLSTPNTAYGGSRAPEERSPLSLHEGLPSLASRDLMGLQR | |
| | PLERMPEGDGEGLSANSSGEQASVPEGNLHGQDQDRGQGPILTL | |
| | NTDAYLSLQELQAQDSVHLI | |
| murine IL7Ra(316-459) | ARDEVESFLPNDLPAQPEELETQGHRAAVHSANRSPETSVSPPET | 92 |
| | VRRESPLRCLARNLSTCNAPPLLSSRSPDYRDGDRNRPPVYQDLLP | |
| | NSGNTNVPVPVPQPLPFQSGILIPVSQRQPISTSSVLNQEEAYVTM | |
| | SSFYQNK | |
| EGFR(955-1186) | VIQGDERMHLPSPTDSNFYRALMDEEDMDDVVDADEYLIPQQG | 93 |
| | FFSSPSTSRTPLLSSLSATSNNSTVACIDRNGLQSCPIKEDSFLQRYS | |
| | SDPTGALTEDSIDDTFLPVPEYINQSVPKRPAGSVQNPVYHNQPL | |
| | NPAPSRDPHYQDPHSTAVGNPEYLNTVQPTCVNSTFDSPAHWA | |
| | QKGSHQISLDNPDYQQDFFPKEAKPNGIFKGSTAENAEYLRVAPQ | |
| | SSEFIGA | |
| EGFR(955- | VIQGDERMHLPSPTDSNFFRALMDEEDMDDVVDADEYLIPQQG | 94 |
| 1186; Y974F, d1045-1057) | FFSSPSTSRTPLLSSLSATSNNSTVACIDRNGLQSCPIKEDSFLQRID | |
| | DTFLPVPEYINQSVPKRPAGSVQNPVYHNQPLNPAPSRDPHYQD | |
| | PHSTAVGNPEYLNTVQPTCVNSTFDSPAHWAQKGSHQISLDNPD | |
| | YQQDFFPKEAKPNGIFKGSTAENAEYLRVAPQSSEFIGA | |
| EGFR(955-1009; Y974F) | VIQGDERMHLPSPTDSNFFRALMDEEDMDDVVDADEYLIPQQG | 95 |
| | FFSSPSTSRTP | |
| EGFR(1019-1085) | NNSTVACIDRNGLQSCPIKEDSFLQRIDDTFLPVPEYINQSVPKRPA | 96 |
| | GSVQNPV | |
| EGFR(1037- | KEDSFLQRIDDTFLPVPEFINQSVPKRPAGSVQNPVYHNQPLNPA | 97 |
| 1103; Y1068/1101F, | PSRDPHFQD | |
| d1045-1057) | | |
| EGFR(1066-1118; | VPEFINQSVPKRPAGSVQNPVFHNQPLNPAPSRDPHYQDPHSTA | 98 |
| Y1068/1086F) | VGNPEYLNTV | |
| EGFR(1122-1165) | PEYLNTVQPTCVNSTFDSPAHWAQKGSHQISLDNPDYQQDFFPK | 99 |
| | EAKPNGIFKG | |
| EGFR(1133-1186; Y1148F) | WAQKGSHQISLDNPDFQQDFFPKEAKPNGIFKGSTAENAEYLRV | 100 |
| | APQSSEFIGA | |
| IL12Rb2(775-825) | SDPKPENPACPWTVLPAGDLPTHDGYLPSNIDDLPSHEAPLADSL | 101 |
| | EELEPQ | |
| IL7R(376-416) | ACDAPILSSSRSLDCRESGKNGPHVYQDLLLSLGTTNSTLP | 102 |
| IL7R(424-459) | GILTLNPVAQGQPILTSLGSNQEEAYVTMSSFYQNQ | 103 |
| IL7R(376-416, 424-459) | ACDAPILSSSRSLDCRESGKNGPHVYQDLLLSLGTTNSTLPQGQPIL | 104 |
| | TSLGSNQEEAYVTMSSFYQNQ | |
| IL7R(424-459; Y456F) | GILTLNPVAQGQPILTSLGSNQEEAYVTMSSFFQNQ | 105 |
| IL7R(376-416, 424- | ACDAPILSSSRSLDCRESGKNGPHVYQDLLLSLGTTNSTLPQGQPIL | 106 |
| 459, Y456F) | TSLGSNQEEAYVTMSSFFQNQ | |
| IL2Rbsmall(393-433) | DEGVAGAPTGSSPQPLQPLSGEDDAYCTFPSRDDLLLFSPS | 107 |
| IL2Rbsmall(518-551) | GQGEFRALNARLPLNTDAYLSLQELQGQDPTHLV | 108 |
| IL2Rbsmall(339-379, 393- | QQDKVPEPASLSSNHSLTSCFTNQGYFFFHLPDALEIEACQDEGV | 109 |
| 433) | AGAPTGSSPQPLQPLSGEDDAYCTFPSRDDLLLFSPS | |
| IL2Rbsmall(339-379, 518- | QQDKVPEPASLSSNHSLTSCFTNQGYFFFHLPDALEIEACQ | 110 |
| 551) | GQGEFRALNARLPLNTDAYLSLQELQGQDPTHLV | |
| IL2Rbsmall(393-433, 518- | DEGVAGAPTGSSPQPLQPLSGEDDAYCTFPSRDDLLLFSPSGQGE | 111 |
| 551) | FRALNARLPLNTDAYLSLQELQGQDPTHLV | |
| IL2Rbsmall(339-379, 393- | QQDKVPEPASLSSNHSLTSCFTNQGYFFFHLPDALEIEACQDEGV | 112 |
| 433, 518-551) | AGAPTGSSPQPLQPLSGEDDAYCTFPSRDDLLLFSPSGQGEFRAL | |
| | NARLPLNTDAYLSLQELQGQDPTHLV | |
| IFNAR2small(310-352) | KKKVWDYNYDDESDSDTEAAPRTSGGGYTMHGLTVRPLGQASA | 113 |
| IFNAR2small(486-515) | EGLWSEDAPSDQSDTSESDVDLGDGYIMR | 114 |
| IFNAR2small(310-352, 486- | KKKVWDYNYDDESDSDTEAAPRTSGGGYTMHGLTVRPLGQASA | 115 |
| 515) | EGLWSEDAPSDQSDTSESDVDLGDGYIMR | |
| BLNK(53-208) | ASESPADEEEQWSDDFDSDYENPDEHSDSEMYVMPAEENADDS | 116 |
| | YEPPPVEQETRPVHPALPFARGEYIDNRSSQRHSPPFSKTLPSKPS | |
| | WPSEKARLTSTLPALTALQKPQVPPKPKGLLEDEADYVVPVEDND | |
| | ENYIHPTESSSPPPEKAPMVNR | |
| BLNK(53-208; Y72F) | ASESPADEEEQWSDDFDSDFENPDEHSDSEMYVMPAEENADDS | 117 |
| | YEPPPVEQETRPVHPALPFARGEYIDNRSSQRHSPPFSKTLPSKPS | |
| | WPSEKARLTSTLPALTALQKPQVPPKPKGLLEDEADYVVPVEDND | |
| | ENYIHPTESSSPPPEKAPMVNR | |
| BLNK(53-208; Y72F, Y96F) | ASESPADEEEQWSDDFDSDFENPDEHSDSEMYVMPAEENADDS | 118 |
| | FEPPPVEQETRPVHPALPFARGEYIDNRSSQRHSPPFSKTLPSKPS | |
| | WPSEKARLTSTLPALTALQKPQVPPKPKGLLEDEADYVVPVEDND | |
| | ENYIHPTESSSPPPEKAPMVNR | |
| EpoR(339-508) | WGTMQAVEPGTDDEGPLLEPVGSEHAQDTYLVLDKWLLPRNPP | 119 |
| | SEDLPGPGGSVDIVAMDEGSEASSCSSALASKPSPEGASAASFEYT | |
| | ILDPSSQLLRPWTLCPELPPTPPHLKYLYLVVSDSGISTDYSSGDSQ | |
| | GAQGGLSDGPYSNPYENSLIPAAEPLPPSYVACS | |
| IL12Rb2(714-862) | VTPVFRHPPCSNWPQREKGIQGHQASEKDMMHSASSPPPPRAL | 120 |
| | QAESRQLVDLYKVLESRGSDPKPENPACPWTVLPAGDLPTHDGYL | |
| | PSNIDDLPSHEAPLADSLEELEPQHISLSVFPSSSLHPLTFSCGDKLT | |
| | LDQLKMRCDSLML | |
| IL12Rb1(622-662) | WDKGERTEPLEKTELPEGAPELALDTELSLEDGDRCKAKM | 121 |
| IL10R1(304-578) | VSPELKNLDLHGSTDSGFGSTKPSLQTEEPQFLLPDPHPQADRTLG | 122 |
| | NREPPVLGDSCSSGSSNSTDSGICLQEPSLSPSTGPTWEQQVGSN | |
| | SRGQDDSGIDLVQNSEGRAGDTQGGSALGHHSPPEPEVPGEEDP | |
| | AAVAFQGYLRQTRCAEEKATKTGCLEEESPLTDGLGPKFGRCLVD | |
| | EAGLHPPALAKGYLKQDPLEMTLASSGAPTGQWNQPTEEWSLLA | |
| | LSSCSDLGISDWSFAHDLAPLGCVAAPGGLLGSFNSDLVTLPLISSL | |
| | QSSE | |
| IL7Ra(316- | ARDEVEGFLQDTFPQQLEESEKQRLGGDVQSPNCPSEDVVITPES | 161 |
| 459).IL12Rb2(775-825) | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |

*SR indicates an exemplary peptide linker

In some embodiments, the Stat-recruiting domain of a chimeric cytokine receptor of the disclosure comprises a STAT-recruiting domain from one receptor.

In order to generate multiple outputs, one or more STAT-recruiting domains may be joined in tandem to mimic signaling from one or more cytokines.

In some embodiments, the STAT-recruiting domain comprises portions of more than one receptor, e.g. comprising more than one STAT-recruiting domain. In such embodiments, a tandem cytokine signaling domain is provided, allowing for enhanced signaling. Accordingly, in some embodiments, the STAT-recruiting domain of a monomer of the chimeric cytokine receptor of the disclosure comprises the STAT-recruiting domains from more than one receptor, e.g. comprises the STAT-recruiting domains from two, three, four, five, or even six receptors. For example, in some embodiments, STAT-recruiting domains can be linked in tandem to stimulate multiple pathways (e.g., the IL7R(316-459)-IL12Rb2(775-825) fragment fusion for pro-persistence STAT5 and pro-inflammatory STAT4; IL7R(316-459)-IL2Rbsmall(393-433,518-551) for pro-persistence; IL7R(316-459)-EGFR(1122-1165) for pro-persistence and anti-exhaustion; IL2Rbsmall(393-433,518-551)-EGFR(1122-1165) for pro-persistence and anti-exhaustion).

When generating multiple outputs, the proximity of individual STAT-recruiting domains to the cell membrane can influence the strength of their respective signaling outputs. Table 5b shows examples of chimeric cytokine receptors with the dual outputs, where each output can be placed either proximal or distal to the cell membrane.

TABLE 5b
Examples of chimeric cytokine receptors with dual outputs

| Dual output STAT- | Membrane | Membrane |
|---|---|---|
| recruiting domain | proximal | distal |
| IL2Rbsmall(393-433, 518-551)/ | IL2Rbsmall(393- | IL21R(322-538) |
| IL21R(322-538) | 433, 518-551) | |
| IL21R(322-538)/ | IL21R(322-538) | IL2Rbsmall(393- |
| IL2Rbsmall(393-433, 518-551) | | 433, 518-551) |
| IL2Rbsmall(339-379, 393- | IL2Rbsmall(339- | IL21R(322-538) |
| 433, 518-551)/IL21R(322-538) | 379, 393-433, | |
| | 518-551) | |
| IL21R(322-538)/ | IL21R(322-538) | IL2Rbsmall(339- |
| IL2Rbsmall(339- | | 379, 393-433, |
| 379, 393-433, 518-551) | | 518-551) |
| IL7R (316-459)/IL21R(322-538) | IL7R (316-459) | IL21R(322-538) |
| IL7R (316-459)/IL12Rb2(775- | IL7R (316-459) | IL12Rb2(775-825) |
| 825) | | |
| IL21R(322-538)/IL7R (316-459) | IL21R(322-538) | IL7R (316-459) |

Without being bound to theory or mechanism, in some embodiments, a JAK-protein (JAK1, JAK2, JAK3, or TYK2) is bound to a chimeric cytokine receptor of the disclosure (comprising a binding domain, a transmembrane domain, a JAK-binding domain, and a recruiting domain). In some embodiments, in the presence of (e.g. binding to) a TGF-β ligand or an anti-TGF-β-receptor antibody, the chimeric cytokine receptor clusters and allows for the two bound JAK-proteins to become activated, which in turn phosphorylate tyrosine residues on the recruiting domain of the chimeric receptor. The phosphorylated recruiting domains are then capable of binding the recruited proteins (e.g. a phosphorylated STAT-recruiting domain binds a STAT-protein), which in turn effectuate transcription events in the nucleus.

### E. Exemplary TGF-β-Driven Chimeric Cytokine Receptors

Context-dependent chimeric cytokine receptors of the disclosure may be expressed with a signal sequence, e.g. a CD8SS of SEQ ID NO: 1. Table 6 shows exemplary context-dependent cytokine receptor sequences of the disclosure. The receptors may be expressed with a signal sequence, e.g. a CD8SS of SEQ ID NO: 1.

In some embodiments, the chimeric cytokine receptor of the disclosure comprises a TGF-β binding domain comprising an amino acid sequence of any one of SEQ ID NOs: 3-20, and 159, a transmembrane and JAK2 binding domain comprising an amino acid sequence of any one of SEQ ID NOs: 27-79, 160 and 217-234, and a recruiting domain comprising an amino acid sequences of any one of SEQ ID NOs: 80-122 and 161. In some embodiments, the chimeric cytokine receptor does not comprise a signal sequence.

In some embodiments, the chimeric cytokine receptor of the disclosure comprises a TGF-β binding domain comprising the amino acid sequence selected from the group consisting of SEQ ID NOs: 3, 4, and 159, a TPOR/MPLR transmembrane and JAK2 binding domain comprising the amino acid sequence selected from the group consisting of SEQ ID NOs: 38, 39, 40, 53, 59, 60, 61, 64, 69, 70, 160 and 217-234, and a recruiting domain comprising the amino acid sequence selected from the group consisting of SEQ ID NOs: 80, 99, 111, 112, and 161. Optionally, the chimeric cytokine receptor comprises a signal sequence that comprises for example the amino acid sequence of SEQ ID NO:1.

In some embodiments, the chimeric cytokine receptor comprises a binding domain comprising the amino acid sequence of SEQ ID NO: 3, 4 or 159, a transmembrane and JAK2 binding domain comprising the amino acid sequence of SEQ ID NO: 64, 69, or 70, and a recruiting domain comprising the amino acid sequence of SEQ ID NO: 80, 99, 111, 112, or 161. In some embodiments, the chimeric cytokine receptor is inducible. In some embodiments, the chimeric cytokine receptor comprises a binding domain comprising the amino acid sequence of SEQ ID NO: 3, 4 or 159, a transmembrane and JAK2 binding domain comprising the amino acid sequence of SEQ ID NO: 38, 39, 40 or 53, and a recruiting domain comprising the amino acid sequence of SEQ ID NO: 80, 99, 111, 112, or 161. In some embodiments, the chimeric cytokine receptor comprises a binding domain comprising the amino acid sequence of SEQ ID NO: 3, 4 or 159, a transmembrane and JAK2 binding domain comprising the amino acid sequence of SEQ ID NO: 59, 60, 160, or 217, and a recruiting domain comprising the amino acid sequence of SEQ ID NO: 80, 99, 111, 112, or 161. In some embodiments, the chimeric cytokine receptor is constitutively active. In some embodiments, the constitutively active chimeric cytokine receptor of the disclosure dimerizes without binding to a TGFβ ligand or an anti-TGFβR antibody. In some embodiments, the chimeric cytokine receptor of the disclosure inhibits TGFβR-mediated signaling and/or activates STAT-mediated signaling, either constitutively or induced by TGF-β, or an anti-TGFβR antibody. In some embodiments, the chimeric cytokine receptor is constitutively active and/or exhibits further enhanced activities or properties in the presence of a TGF-βR ligand, e.g., TGF-β, or an anti-TGF-βR antibody. In some embodiments, the TGF-βR is TGF-βR2, and the antibody is an anti-TGF-βR2 antibody.

In some embodiments, the chimeric cytokine receptor does not comprise a signal sequence. In some embodiments, the chimeric cytokine receptor comprises the TGFβR2 endogenous signal sequence or a signal sequence that comprise, e.g., the amino acid sequence of SEQ ID NO:1.

In some embodiments, the chimeric cytokine receptor of the disclosure comprises the amino acid sequence of SEQ ID NO: 123. In some embodiments, the chimeric cytokine receptor of the disclosure comprises the amino acid sequence of SEQ ID NO: 124. In some embodiments, the chimeric cytokine receptor of the disclosure comprises the amino acid sequence of SEQ ID NO: 125. In some embodiments, the chimeric cytokine receptor of the disclosure comprises the amino acid sequence of SEQ ID NO: 126. In some embodiments, the chimeric cytokine receptor of the disclosure comprises the amino acid sequence of SEQ ID NO: 127. In some embodiments, the chimeric cytokine receptor of the disclosure comprises the amino acid sequence of SEQ ID NO: 128. In some embodiments, the chimeric cytokine receptor of the disclosure comprises the amino acid sequence of SEQ ID NO: 129. In some embodiments, the chimeric cytokine receptor of the disclosure comprises the amino acid sequence of SEQ ID NO: 130. In some embodiments, the chimeric cytokine receptor of the disclosure comprises the amino acid sequence of SEQ ID NO: 131. In some embodiments, the chimeric cytokine receptor of the disclosure comprises the amino acid sequence of SEQ ID NO: 132. In some embodiments, the chimeric cytokine receptor of the disclosure comprises the amino acid sequence of SEQ ID NO: 133. In some embodiments, the chimeric cytokine receptor of the disclosure comprises the amino acid sequence of SEQ ID NO: 134. In some embodiments, the chimeric cytokine receptor of the disclosure comprises the amino acid sequence of SEQ ID NO: 135. In some embodiments, the chimeric cytokine receptor of the disclosure comprises the amino acid sequence of SEQ ID NO: 136. In some embodiments, the chimeric cytokine receptor of the disclosure comprises the amino acid sequence of SEQ ID NO: 137. In some embodiments, the chimeric cytokine receptor of the disclosure comprises the amino acid sequence of SEQ ID NO: 138. In some embodiments, the chimeric cytokine receptor of the disclosure comprises the amino acid sequence of SEQ ID NO: 139. In some embodiments, the chimeric cytokine receptor of the disclosure comprises the amino acid sequence of SEQ ID NO: 140. In some embodiments, the chimeric cytokine receptor of the disclosure comprises the amino acid sequence of SEQ ID NO: 141. In some embodiments, the chimeric cytokine receptor of the disclosure comprises the amino acid sequence of SEQ ID NO: 142. In some embodiments, the chimeric cytokine receptor of the disclosure comprises the amino acid sequence of SEQ ID NO: 143. In some embodiments, the chimeric cytokine receptor of the disclosure comprises the amino acid sequence of SEQ ID NO: 144. In some embodiments, the chimeric cytokine receptor of the disclosure comprises the amino acid sequence of SEQ ID NO: 145. In some embodiments, the chimeric cytokine receptor of the disclosure comprises the amino acid sequence of SEQ ID NO: 146. In some embodiments, the chimeric cytokine receptor of the disclosure comprises the amino acid sequence of SEQ ID NO: 147. In some embodiments, the chimeric cytokine receptor of the disclosure comprises the amino acid sequence of SEQ ID NO: 148. In some embodiments, the chimeric cytokine receptor of the disclosure comprises the amino acid sequence of SEQ ID NO: 149. In some embodiments, the chimeric cytokine receptor of the disclosure comprises the amino acid sequence of SEQ ID NO: 150. In some embodiments, the chimeric cytokine receptor of the disclosure comprises the amino acid sequence of SEQ ID NO: 151. In some embodiments, the chimeric cytokine receptor of the disclosure comprises the amino acid sequence of SEQ ID NO: 162. In some embodiments, the chimeric cytokine receptor of the disclosure comprises the amino acid sequence of SEQ ID NO: 163. In some embodiments, the chimeric cytokine receptor of the disclosure comprises the amino acid sequence of SEQ ID NO: 164. In some embodiments, the chimeric cytokine receptor of the disclosure comprises the amino acid sequence of SEQ ID NO:165. In some embodiments, the chimeric cytokine receptor of the disclosure comprises the amino acid sequence of SEQ ID NO:166. In some embodiments, the chimeric cytokine receptor of the disclosure comprises the amino acid sequence of SEQ ID NO:167. In some embodiments, the chimeric cytokine receptor of the disclosure comprises the amino acid sequence of SEQ ID NO:168. In some embodiments, the chimeric cytokine receptor of the disclosure comprises the amino acid sequence of SEQ ID NO:169. In some embodiments, the chimeric cytokine receptor of the disclosure comprises the amino acid sequence of SEQ ID NO:170. In some embodiments, the chimeric cytokine receptor of the disclosure comprises the amino acid sequence of SEQ ID NO:171. In some embodiments, the chimeric cytokine receptor of the disclosure comprises the amino acid sequence of SEQ ID NO:172. In some embodiments, the chimeric cytokine receptor of the disclosure comprises the amino acid sequence of SEQ ID NO:173. In some embodiments, the chimeric cytokine receptor of the disclosure comprises the amino acid sequence of SEQ ID NO:174. In some embodiments, the chimeric cytokine receptor of the disclosure comprises the amino acid sequence of SEQ ID NO:175. In some embodiments, the chimeric cytokine receptor of the disclosure comprises the amino acid sequence of SEQ ID NO:176. In some embodiments, the chimeric cytokine receptor of the disclosure comprises the amino acid sequence of SEQ ID NO:177. In some embodiments, the chimeric cytokine receptor of the disclosure comprises the amino acid sequence of SEQ ID NO:178. In some embodiments, the chimeric cytokine receptor of the disclosure comprises the amino acid sequence of SEQ ID NO:179. In some embodiments, the chimeric cytokine receptor of the disclosure comprises the amino acid sequence of SEQ ID NO:180. In some embodiments, the chimeric cytokine receptor of the disclosure comprises the amino acid sequence of SEQ ID NO:181. In some embodiments, the chimeric cytokine receptor of the disclosure comprises the amino acid sequence of SEQ ID NO:182. In some embodiments, the chimeric cytokine receptor of the disclosure comprises the amino acid sequence of SEQ ID NO:183. In some embodiments, the chimeric cytokine receptor of the disclosure comprises the amino acid sequence of SEQ ID NO:184. In some embodiments, the chimeric cytokine receptor of the disclosure comprises the amino acid sequence of SEQ ID NO:185. In some embodiments, the chimeric cytokine receptor of the disclosure comprises the amino acid sequence of SEQ ID NO:186. In some embodiments, the chimeric cytokine receptor of the disclosure comprises the amino acid sequence of SEQ ID NO:187. In some embodiments, the chimeric cytokine receptor of the disclosure comprises the amino acid sequence of SEQ ID NO:188. In some embodiments, the chimeric cytokine receptor of the disclosure comprises the amino acid sequence of SEQ ID NO:189. In some embodiments, the chimeric cytokine receptor of the disclosure comprises the amino acid sequence of SEQ ID NO:190. In some embodiments, the chimeric cytokine receptor of the disclosure comprises the amino acid sequence of SEQ ID NO:191. In some embodiments, the chimeric cytokine receptor of the disclosure comprises the amino acid sequence of SEQ ID NO:192. In some embodiments, the chimeric cytokine receptor of the disclosure comprises the amino acid sequence of SEQ ID NO:193. In some embodiments, the chimeric cytokine receptor of the disclosure comprises the amino acid sequence of SEQ ID NO:194. In some embodiments, the chimeric cytokine receptor of the disclosure comprises the amino acid sequence of SEQ ID NO:195. In some embodiments, the chimeric cytokine receptor of the disclosure comprises the amino acid sequence of SEQ ID NO:196. In some embodiments, the chimeric cytokine receptor of the disclosure comprises the amino acid sequence of SEQ ID NO:197. In some embodiments, the chimeric cytokine receptor of the disclosure comprises the amino acid sequence of SEQ ID NO:198. In some embodiments, the chimeric cytokine receptor of the disclosure comprises the amino acid sequence of SEQ ID NO:199. In some embodiments, the chimeric cytokine receptor of the disclosure comprises the amino acid sequence of SEQ ID NO:200. In some embodiments, the chimeric cytokine receptor of the disclosure comprises the amino acid sequence of SEQ ID NO:201. In some embodiments, the chimeric cytokine receptor of the disclosure comprises the amino acid sequence of SEQ ID NO:202. In some embodiments, the chimeric cytokine receptor of the disclosure comprises the amino acid sequence of SEQ ID NO:203. In some embodiments, the chimeric cytokine receptor of the disclosure comprises the amino acid sequence of SEQ ID NO:204. In some embodiments, the chimeric cytokine receptor of the disclosure comprises the amino acid sequence of SEQ ID NO:205. In some embodiments, the chimeric cytokine receptor of the disclosure comprises the amino acid sequence of SEQ ID NO:206. In some embodiments, the chimeric cytokine receptor of the disclosure comprises the amino acid sequence of SEQ ID NO:207. In some embodiments, the chimeric cytokine receptor of the disclosure comprises the amino acid sequence of SEQ ID NO:208. In some embodiments, the chimeric cytokine receptor of the disclosure comprises the amino acid sequence of SEQ ID NO:209. In some embodiments, the chimeric cytokine receptor of the disclosure comprises the amino acid sequence of SEQ ID NO:210. In some embodiments, the chimeric cytokine receptor of the disclosure comprises the amino acid sequence of SEQ ID NO:211. In some embodiments, the chimeric cytokine receptor of the disclosure comprises the amino acid sequence of SEQ ID NO:212. In some embodiments, the chimeric cytokine receptor of the disclosure comprises the amino acid sequence of SEQ ID NO:213. In some embodiments, the chimeric cytokine receptor of the disclosure comprises the amino acid sequence of SEQ ID NO:214. In some embodiments, the chimeric cytokine receptor of the disclosure comprises the amino acid sequence of SEQ ID NO:215. In some embodiments, the chimeric cytokine receptor of the disclosure comprises the amino acid sequence of SEQ ID NO:216.

In some embodiments, the chimeric cytokine receptor (CCR) comprises a binding domain comprising the amino acid sequence of SEQ ID NO: 159, a transmembrane and JAK2 binding domain comprising the amino acid sequence of SEQ ID NO: 40, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 53, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 38, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 39, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 40, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 53, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 70, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 69, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 64, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 69, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 70, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 160 or 219, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 223, 224, and a recruiting domain comprising the amino acid sequence of SEQ ID NO: 80, 99, 111, 112, or 161. In some embodiments, the CCR comprises a binding domain comprising the amino acid sequence of SEQ ID NO: 159, a transmembrane and JAK2 binding domain comprising the amino acid sequence of SEQ ID NO: 225 or 226, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 60 or 160, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 223, 224, and a recruiting domain comprising the amino acid sequence of SEQ ID NO: 80, 99, 111, 112, or 161. In some embodiments, the CCR comprises a binding domain comprising the amino acid sequence of SEQ ID NO: 4, a transmembrane and JAK2 binding domain comprising the amino acid sequence of SEQ ID NO: 225 or 226, and a recruiting domain comprising the amino acid sequence of SEQ ID NO: 80, 99, 111, 112, or 161

In some embodiments, the chimeric cytokine receptor of the disclosure comprises the amino acid sequence of SEQ ID NO:272, SEQ ID NO:273, SEQ ID NO:274, or SEQ ID NO:275. In some embodiments, the chimeric cytokine receptor comprises an amino acid sequence that is at least 80%, 85%, 90%, 95%, 98%, or 99%, or 100% identical to any one of the amino acid sequences of SEQ ID NOs: 123-216 and SEQ ID NOs: 272-275.

TABLE 6
Exemplary chimeric cytokine receptor sequences (assembled inducible or
constitutively active TGF-β receptor chimeric cytokine receptors):

| | | SEQ ID |
|---|---|---|
| Receptor | Amino acid sequence | NO: |
| CD8SS-TGFbR2(1- | MALPVTALLLPLALLLHAARPMGRGLLRGLWPLHIVLWTRIASTIP | 123 |
| 166).TPOR/MPLR(478- | PHVQKSVNNDMIVTDNNGAVKFPQLCKFCDVRFSTCDNQKSCM | |
| 582).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWISLVTALHLVLGLSAVLGLLLLRWQFPA | |
| | HYRRLRHALWPSLPDLHRVLGQYLRDTAALSPPKATVSDTCEEVE | |
| | PSLLEILPKSSERTPLPLARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 124 |
| 166).TPOR/MPLR(478- | PHVQKSVNNDMIVTDNNGAVKFPQLCKFCDVRFSTCDNQKSCM | |
| 582; N - 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETWISLVTALHLVLGLSAVLGLLLLRWQFPAH | |
| | YRRLRHALWPSLPDLHRVLGQYLRDTAALSPPKATVSDTCEEVEPS | |
| | LLEILPKSSERTPLPLARDEVEGFLQDTFPQQLEESEKQRLGGDVQS | |
| | PNCPSEDVVITPESFGRDSSLTCLAGNVSACDAPILSSSRSLDCRES | |
| | GKNGPHVYQDLLLSLGTTNSTLPPPFSLQSGILTLNPVAQGQPILTS | |
| | LGSNQEEAYVTMSSFYQNQSDPKPENPACPWTVLPAGDLPTHD | |
| | GYLPSNIDDLPSHEAPLADSLEELEPQ | |
| CD8SS-TGFbR2(1- | MALPVTALLLPLALLLHAARPMGRGLLRGLWPLHIVLWTRIASTIP | 125 |
| 166).TPOR/MPLR(478- | PHVQKSVNNDMIVTDNNGAVKFPQLCKFCDVRFSTCDNQKSCM | |
| 582; N - 2).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETISLVTALHLVLGLSAVLGLLLLRWQFPAHYR | |
| | RLRHALWPSLPDLHRVLGQYLRDTAALSPPKATVSDTCEEVEPSLL | |
| | EILPKSSERTPLPLARDEVEGFLQDTFPQQLEESEKQRLGGDVQSP | |
| | NCPSEDVVITPESFGRDSSLTCLAGNVSACDAPILSSSRSLDCRESG | |
| | KNGPHVYQDLLLSLGTTNSTLPPPFSLQSGILTLNPVAQGQPILTSL | |
| | GSNQEEAYVTMSSFYQNQSDPKPENPACPWTVLPAGDLPTHDG | |
| | YLPSNIDDLPSHEAPLADSLEELEPQ | |
| CD8SS-TGFbR2(1- | MALPVTALLLPLALLLHAARPMGRGLLRGLWPLHIVLWTRIASTIP | 126 |
| 166).TPOR/MPLR(478- | PHVQKSVNNDMIVTDNNGAVKFPQLCKFCDVRFSTCDNQKSCM | |
| 582; N - 2 + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLISLVTALHLVLGLSAVLGLLLLRWQFPAHY | |
| | RRLRHALWPSLPDLHRVLGQYLRDTAALSPPKATVSDTCEEVEPSL | |
| | LEILPKSSERTPLPLARDEVEGFLQDTFPQQLEESEKQRLGGDVQS | |
| | PNCPSEDVVITPESFGRDSSLTCLAGNVSACDAPILSSSRSLDCRES | |
| | GKNGPHVYQDLLLSLGTTNSTLPPPFSLQSGILTLNPVAQGQPILTS | |
| | LGSNQEEAYVTMSSFYQNQSDPKPENPACPWTVLPAGDLPTHD | |
| | GYLPSNIDDLPSHEAPLADSLEELEPQ | |
| CD8SS-TGFbR2(1- | MALPVTALLLPLALLLHAARPMGRGLLRGLWPLHIVLWTRIASTIP | 127 |
| 166).TPOR/MPLR(478- | PHVQKSVNNDMIVTDNNGAVKFPQLCKFCDVRFSTCDNQKSCM | |
| 582; N - 3).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETSLVTALHLVLGLSAVLGLLLLRWQFPAHYRR | |
| | LRHALWPSLPDLHRVLGQYLRDTAALSPPKATVSDTCEEVEPSLLEI | |
| | LPKSSERTPLPLARDEVEGFLQDTFPQQLEESEKQRLGGDVQSPN | |
| | CPSEDVVITPESFGRDSSLTCLAGNVSACDAPILSSSRSLDCRESGK | |
| | NGPHVYQDLLLSLGTTNSTLPPPFSLQSGILTLNPVAQGQPILTSLG | |
| | SNQEEAYVTMSSFYQNQSDPKPENPACPWTVLPAGDLPTHDGY | |
| | LPSNIDDLPSHEAPLADSLEELEPQ | |
| CD8SS-TGFbR2(1- | MALPVTALLLPLALLLHAARPMGRGLLRGLWPLHIVLWTRIASTIP | 128 |
| 166).TPOR/MPLR(478- | PHVQKSVNNDMIVTDNNGAVKFPQLCKFCDVRFSTCDNQKSCM | |
| 582; N - 4).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLVTALHLVLGLSAVLGLLLLRWQFPAHYRRL | |
| | RHALWPSLPDLHRVLGQYLRDTAALSPPKATVSDTCEEVEPSLLEIL | |
| | PKSSERTPLPLARDEVEGFLQDTFPQQLEESEKQRLGGDVQSPNC | |
| | PSEDVVITPESFGRDSSLTCLAGNVSACDAPILSSSRSLDCRESGKN | |
| | GPHVYQDLLLSLGTTNSTLPPPFSLQSGILTLNPVAQGQPILTSLGS | |
| | NQEEAYVTMSSFYQNQSDPKPENPACPWTVLPAGDLPTHDGYL | |
| | PSNIDDLPSHEAPLADSLEELEPQ | |
| CD8SS-TGFbR2(1- | MALPVTALLLPLALLLHAARPMGRGLLRGLWPLHIVLWTRIASTIP | 129 |
| 166).TPOR/MPLR(478- | PHVQKSVNNDMIVTDNNGAVKFPQLCKFCDVRFSTCDNQKSCM | |
| 582; N - 4 + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETILVTALHLVLGLSAVLGLLLLRWQFPAHYRR | |
| | LRHALWPSLPDLHRVLGQYLRDTAALSPPKATVSDTCEEVEPSLLEI | |
| | LPKSSERTPLPLARDEVEGFLQDTFPQQLEESEKQRLGGDVQSPN | |
| | CPSEDVVITPESFGRDSSLTCLAGNVSACDAPILSSSRSLDCRESGK | |
| | NGPHVYQDLLLSLGTTNSTLPPPFSLQSGILTLNPVAQGQPILTSLG | |
| | SNQEEAYVTMSSFYQNQSDPKPENPACPWTVLPAGDLPTHDGY | |
| | LPSNIDDLPSHEAPLADSLEELEPQ | |
| CD8SS-TGFbR2(1- | MALPVTALLLPLALLLHAARPMGRGLLRGLWPLHIVLWTRIASTIP | 130 |
| 166).TPOR/MPLR(478- | PHVQKSVNNDMIVTDNNGAVKFPQLCKFCDVRFSTCDNQKSCM | |
| 582; N - 5).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETVTALHLVLGLSAVLGLLLLRWQFPAHYRRL | |
| | RHALWPSLPDLHRVLGQYLRDTAALSPPKATVSDTCEEVEPSLLEIL | |
| | PKSSERTPLPLARDEVEGFLQDTFPQQLEESEKQRLGGDVQSPNC | |
| | PSEDVVITPESFGRDSSLTCLAGNVSACDAPILSSSRSLDCRESGKN | |
| | GPHVYQDLLLSLGTTNSTLPPPFSLQSGILTLNPVAQGQPILTSLGS | |
| | NQEEAYVTMSSFYQNQSDPKPENPACPWTVLPAGDLPTHDGYL | |
| | PSNIDDLPSHEAPLADSLEELEPQ | |
| CD8SS-TGFbR2(1- | MALPVTALLLPLALLLHAARPMGRGLLRGLWPLHIVLWTRIASTIP | 131 |
| 166).TPOR/MPLR(478- | PHVQKSVNNDMIVTDNNGAVKFPQLCKFCDVRFSTCDNQKSCM | |
| 582; N - 6).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETTALHLVLGLSAVLGLLLLRWQFPAHYRRLR | |
| | HALWPSLPDLHRVLGQYLRDTAALSPPKATVSDTCEEVEPSLLEILP | |
| | KSSERTPLPLARDEVEGFLQDTFPQQLEESEKQRLGGDVQSPNCP | |
| | SEDVVITPESFGRDSSLTCLAGNVSACDAPILSSSRSLDCRESGKNG | |
| | PHVYQDLLLSLGTTNSTLPPPFSLQSGILTLNPVAQGQPILTSLGSN | |
| | QEEAYVTMSSFYQNQSDPKPENPACPWTVLPAGDLPTHDGYLPS | |
| | NIDDLPSHEAPLADSLEELEPQ | |
| CD8SS-TGFbR2(1- | MALPVTALLLPLALLLHAARPMGRGLLRGLWPLHIVLWTRIASTIP | 132 |
| 166).TPOR/MPLR(478- | PHVQKSVNNDMIVTDNNGAVKFPQLCKFCDVRFSTCDNQKSCM | |
| 582; N - 7).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETALHLVLGLSAVLGLLLLRWQFPAHYRRLRH | |
| | ALWPSLPDLHRVLGQYLRDTAALSPPKATVSDTCEEVEPSLLEILPK | |
| | SSERTPLPLARDEVEGFLQDTFPQQLEESEKQRLGGDVQSPNCPS | |
| | EDVVITPESFGRDSSLTCLAGNVSACDAPILSSSRSLDCRESGKNGP | |
| | HVYQDLLLSLGTTNSTLPPPFSLQSGILTLNPVAQGQPILTSLGSNQ | |
| | EEAYVTMSSFYQNQSDPKPENPACPWTVLPAGDLPTHDGYLPSN | |
| | IDDLPSHEAPLADSLEELEPQ | |
| CD8SS-TGFbR2(1- | MALPVTALLLPLALLLHAARPMGRGLLRGLWPLHIVLWTRIASTIP | 133 |
| 166).TPOR/MPLR(478- | PHVQKSVNNDMIVTDNNGAVKFPQLCKFCDVRFSTCDNQKSCM | |
| 582; N - 8).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLHLVLGLSAVLGLLLLRWQFPAHYRRLRHA | |
| | LWPSLPDLHRVLGQYLRDTAALSPPKATVSDTCEEVEPSLLEILPKS | |
| | SERTPLPLARDEVEGFLQDTFPQQLEESEKQRLGGDVQSPNCPSE | |
| | DVVITPESFGRDSSLTCLAGNVSACDAPILSSSRSLDCRESGKNGPH | |
| | VYQDLLLSLGTTNSTLPPPFSLQSGILTLNPVAQGQPILTSLGSNQE | |
| | EAYVTMSSFYQNQSDPKPENPACPWTVLPAGDLPTHDGYLPSNI | |
| | DDLPSHEAPLADSLEELEPQ | |
| CD8SS-TGFbR2(1- | MALPVTALLLPLALLLHAARPMGRGLLRGLWPLHIVLWTRIASTIP | 134 |
| 166).TPOR/MPLR(478- | PHVQKSVNNDMIVTDNNGAVKFPQLCKFCDVRFSTCDNQKSCM | |
| 582; N - 9).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETHLVLGLSAVLGLLLLRWQFPAHYRRLRHAL | |
| | WPSLPDLHRVLGQYLRDTAALSPPKATVSDTCEEVEPSLLEILPKSS | |
| | ERTPLPLARDEVEGFLQDTFPQQLEESEKQRLGGDVQSPNCPSED | |
| | VVITPESFGRDSSLTCLAGNVSACDAPILSSSRSLDCRESGKNGPHV | |
| | YQDLLLSLGTTNSTLPPPFSLQSGILTLNPVAQGQPILTSLGSNQEE | |
| | AYVTMSSFYQNQSDPKPENPACPWTVLPAGDLPTHDGYLPSNID | |
| | DLPSHEAPLADSLEELEPQ | |
| CD8SS-TGFbR2(1- | MALPVTALLLPLALLLHAARPMGRGLLRGLWPLHIVLWTRIASTIP | 135 |
| 166).TPOR/MPLR(478- | PHVQKSVNNDMIVTDNNGAVKFPQLCKFCDVRFSTCDNQKSCM | |
| 582; N - 10).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLVLGLSAVLGLLLLRWQFPAHYRRLRHALW | |
| | PSLPDLHRVLGQYLRDTAALSPPKATVSDTCEEVEPSLLEILPKSSER | |
| | TPLPLARDEVEGFLQDTFPQQLEESEKQRLGGDVQSPNCPSEDVV | |
| | ITPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 136 |
| 166).TPOR/MPLR(478- | PHVQKSVNNDMIVTDNNGAVKFPQLCKFCDVRFSTCDNQKSCM | |
| 582; N - 11).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETVLGLSAVLGLLLLRWQFPAHYRRLRHALW | |
| | PSLPDLHRVLGQYLRDTAALSPPKATVSDTCEEVEPSLLEILPKSSER | |
| | TPLPLARDEVEGFLQDTFPQQLEESEKQRLGGDVQSPNCPSEDVV | |
| | ITPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 137 |
| 166).TPOR/MPLR(478- | PHVQKSVNNDMIVTDNNGAVKFPQLCKFCDVRFSTCDNQKSCM | |
| 582; N - 12).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLGLSAVLGLLLLRWQFPAHYRRLRHALWPS | |
| | LPDLHRVLGQYLRDTAALSPPKATVSDTCEEVEPSLLEILPKSSERTP | |
| | LPLARDEVEGFLQDTFPQQLEESEKQRLGGDVQSPNCPSEDVVIT | |
| | PESFGRDSSLTCLAGNVSACDAPILSSSRSLDCRESGKNGPHVYQD | |
| | LLLSLGTTNSTLPPPFSLQSGILTLNPVAQGQPILTSLGSNQEEAYVT | |
| | MSSFYQNQSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 138 |
| 166).TPOR/MPLR(478- | PHVQKSVNNDMIVTDNNGAVKFPQLCKFCDVRFSTCDNQKSCM | |
| 582; N - 13).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETGLSAVLGLLLLRWQFPAHYRRLRHALWPSL | |
| | PDLHRVLGQYLRDTAALSPPKATVSDTCEEVEPSLLEILPKSSERTPL | |
| | PLARDEVEGFLQDTFPQQLEESEKQRLGGDVQSPNCPSEDVVITP | |
| | ESFGRDSSLTCLAGNVSACDAPILSSSRSLDCRESGKNGPHVYQDL | |
| | LLSLGTTNSTLPPPFSLQSGILTLNPVAQGQPILTSLGSNQEEAYVT | |
| | MSSFYQNQSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 139 |
| 166).TPOR/MPLR(478- | PHVQKSVNNDMIVTDNNGAVKFPQLCKFCDVRFSTCDNQKSCM | |
| 582; N - 14).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLSAVLGLLLLRWQFPAHYRRLRHALWPSLP | |
| | DLHRVLGQYLRDTAALSPPKATVSDTCEEVEPSLLEILPKSSERTPLP | |
| | LARDEVEGFLQDTFPQQLEESEKQRLGGDVQSPNCPSEDVVITPE | |
| | SFGRDSSLTCLAGNVSACDAPILSSSRSLDCRESGKNGPHVYQDLL | |
| | LSLGTTNSTLPPPFSLQSGILTLNPVAQGQPILTSLGSNQEEAYVTM | |
| | SSFYQNQSDPKPENPACPWTVLPAGDLPTHDGYLPSNIDDLPSHE | |
| | APLADSLEELEPQ | |
| CD8SS-TGFbR2(1- | MALPVTALLLPLALLLHAARPMGRGLLRGLWPLHIVLWTRIASTIP | 140 |
| 166).TPOR/MPLR(478- | PHVQKSVNNDMIVTDNNGAVKFPQLCKFCDVRFSTCDNQKSCM | |
| 582; N - 15).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETSAVLGLLLLRWQFPAHYRRLRHALWPSLPD | |
| | LHRVLGQYLRDTAALSPPKATVSDTCEEVEPSLLEILPKSSERTPLPL | |
| | ARDEVEGFLQDTFPQQLEESEKQRLGGDVQSPNCPSEDVVITPES | |
| | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQSDPKPENPACPWTVLPAGDLPTHDGYLPSNIDDLPSHE | |
| | APLADSLEELEPQ | |
| CD8SS-TGFbR2(1- | MALPVTALLLPLALLLHAARPMGRGLLRGLWPLHIVLWTRIASTIP | 141 |
| 166).TPOR/MPLR(478- | PHVQKSVNNDMIVTDNNGAVKFPQLCKFCDVRFSTCDNQKSCM | |
| 582; N - 16).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAVLGLLLLRWQFPAHYRRLRHALWPSLPDL | |
| | HRVLGQYLRDTAALSPPKATVSDTCEEVEPSLLEILPKSSERTPLPLA | |
| | RDEVEGFLQDTFPQQLEESEKQRLGGDVQSPNCPSEDVVITPESF | |
| | GRDSSLTCLAGNVSACDAPILSSSRSLDCRESGKNGPHVYQDLLLS | |
| | LGTTNSTLPPPFSLQSGILTLNPVAQGQPILTSLGSNQEEAYVTMS | |
| | SFYQNQSDPKPENPACPWTVLPAGDLPTHDGYLPSNIDDLPSHE | |
| | APLADSLEELEPQ | |
| CD8SS-TGFbR2(1- | MALPVTALLLPLALLLHAARPMGRGLLRGLWPLHIVLWTRIASTIP | 142 |
| 166).TPOR/MPLR(478- | PHVQKSVNNDMIVTDNNGAVKFPQLCKFCDVRFSTCDNQKSCM | |
| 582; N - 17).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETVLGLLLLRWQFPAHYRRLRHALWPSLPDLH | |
| | RVLGQYLRDTAALSPPKATVSDTCEEVEPSLLEILPKSSERTPLPLAR | |
| | DEVEGFLQDTFPQQLEESEKQRLGGDVQSPNCPSEDVVITPESFG | |
| | RDSSLTCLAGNVSACDAPILSSSRSLDCRESGKNGPHVYQDLLLSL | |
| | GTTNSTLPPPFSLQSGILTLNPVAQGQPILTSLGSNQEEAYVTMSS | |
| | FYQNQSDPKPENPACPWTVLPAGDLPTHDGYLPSNIDDLPSHEA | |
| | PLADSLEELEPQ | |
| CD8SS-TGFbR2(1- | MALPVTALLLPLALLLHAARPMGRGLLRGLWPLHIVLWTRIASTIP | 143 |
| 166).TPOR/MPLR(478- | PHVQKSVNNDMIVTDNNGAVKFPQLCKFCDVRFSTCDNQKSCM | |
| 582; N - 18).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLGLLLLRWQFPAHYRRLRHALWPSLPDLHR | |
| | VLGQYLRDTAALSPPKATVSDTCEEVEPSLLEILPKSSERTPLPLARD | |
| | EVEGFLQDTFPQQLEESEKQRLGGDVQSPNCPSEDVVITPESFGR | |
| | DSSLTCLAGNVSACDAPILSSSRSLDCRESGKNGPHVYQDLLLSLGT | |
| | TNSTLPPPFSLQSGILTLNPVAQGQPILTSLGSNQEEAYVTMSSFY | |
| | QNQSDPKPENPACPWTVLPAGDLPTHDGYLPSNIDDLPSHEAPL | |
| | ADSLEELEPQ | |
| CD8SS-TGFbR2(1- | MALPVTALLLPLALLLHAARPMGRGLLRGLWPLHIVLWTRIASTIP | 144 |
| 166).TPOR/MPLR(478- | PHVQKSVNNDMIVTDNNGAVKFPQLCKFCDVRFSTCDNQKSCM | |
| 582; N + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLISLVTALHLVLGLSAVLGLLLLRWQFPA | |
| | HYRRLRHALWPSLPDLHRVLGQYLRDTAALSPPKATVSDTCEEVE | |
| | PSLLEILPKSSERTPLPLARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 145 |
| 166).TPOR/MPLR(478- | PHVQKSVNNDMIVTDNNGAVKFPQLCKFCDVRFSTCDNQKSCM | |
| 582; N + 2).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWVLISLVTALHLVLGLSAVLGLLLLRWQFP | |
| | AHYRRLRHALWPSLPDLHRVLGQYLRDTAALSPPKATVSDTCEEV | |
| | EPSLLEILPKSSERTPLPLARDEVEGFLQDTFPQQLEESEKQRLGGD | |
| | VQSPNCPSEDVVITPESFGRDSSLTCLAGNVSACDAPILSSSRSLDC | |
| | RESGKNGPHVYQDLLLSLGTTNSTLPPPFSLQSGILTLNPVAQGQP | |
| | ILTSLGSNQEEAYVTMSSFYQNQSDPKPENPACPWTVLPAGDLPT | |
| | HDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 146 |
| 166).TPOR/MPLR(478- | PHVQKSVNNDMIVTDNNGAVKFPQLCKFCDVRFSTCDNQKSCM | |
| 582; N + 3).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLVLISLVTALHLVLGLSAVLGLLLLRWQF | |
| | PAHYRRLRHALWPSLPDLHRVLGQYLRDTAALSPPKATVSDTCEE | |
| | VEPSLLEILPKSSERTPLPLARDEVEGFLQDTFPQQLEESEKQRLGG | |
| | DVQSPNCPSEDVVITPESFGRDSSLTCLAGNVSACDAPILSSSRSLD | |
| | CRESGKNGPHVYQDLLLSLGTTNSTLPPPFSLQSGILTLNPVAQGQ | |
| | PILTSLGSNQEEAYVTMSSFYQNQSDPKPENPACPWTVLPAGDLP | |
| | THDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 147 |
| 166).TPOR/MPLR(478- | PHVQKSVNNDMIVTDNNGAVKFPQLCKFCDVRFSTCDNQKSCM | |
| 582; N + 4).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWILVLISLVTALHLVLGLSAVLGLLLLRWQ | |
| | FPAHYRRLRHALWPSLPDLHRVLGQYLRDTAALSPPKATVSDTCE | |
| | EVEPSLLEILPKSSERTPLPLARDEVEGFLQDTFPQQLEESEKQRLG | |
| | GDVQSPNCPSEDVVITPESFGRDSSLTCLAGNVSACDAPILSSSRSL | |
| | DCRESGKNGPHVYQDLLLSLGTTNSTLPPPFSLQSGILTLNPVAQG | |
| | QPILTSLGSNQEEAYVTMSSFYQNQSDPKPENPACPWTVLPAGD | |
| | LPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 148 |
| 166).TPOR/MPLR(478- | PHVQKSVNNDMIVTDNNGAVKFPQLCKFCDVRFSTCDNQKSCM | |
| 582; N + 5).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLILVLISLVTALHLVLGLSAVLGLLLLRW | |
| | QFPAHYRRLRHALWPSLPDLHRVLGQYLRDTAALSPPKATVSDTC | |
| | EEVEPSLLEILPKSSERTPLPLARDEVEGFLQDTFPQQLEESEKQRLG | |
| | GDVQSPNCPSEDVVITPESFGRDSSLTCLAGNVSACDAPILSSSRSL | |
| | DCRESGKNGPHVYQDLLLSLGTTNSTLPPPFSLQSGILTLNPVAQG | |
| | QPILTSLGSNQEEAYVTMSSFYQNQSDPKPENPACPWTVLPAGD | |
| | LPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 149 |
| 166).TPOR/MPLR(478- | PHVQKSVNNDMIVTDNNGAVKFPQLCKFCDVRFSTCDNQKSCM | |
| 582; N + 6).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLLILVLISLVTALHLVLGLSAVLGLLLLRW | |
| | QFPAHYRRLRHALWPSLPDLHRVLGQYLRDTAALSPPKATVSDTC | |
| | EEVEPSLLEILPKSSERTPLPLARDEVEGFLQDTFPQQLEESEKQRLG | |
| | GDVQSPNCPSEDVVITPESFGRDSSLTCLAGNVSACDAPILSSSRSL | |
| | DCRESGKNGPHVYQDLLLSLGTTNSTLPPPFSLQSGILTLNPVAQG | |
| | QPILTSLGSNQEEAYVTMSSFYQNQSDPKPENPACPWTVLPAGD | |
| | LPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 150 |
| 166).TPOR/MPLR(478- | PHVQKSVNNDMIVTDNNGAVKFPQLCKFCDVRFSTCDNQKSCM | |
| 582; N + 7).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWVLLILVLISLVTALHLVLGLSAVLGLLLLR | |
| | WQFPAHYRRLRHALWPSLPDLHRVLGQYLRDTAALSPPKATVSD | |
| | TCEEVEPSLLEILPKSSERTPLPLARDEVEGFLQDTFPQQLEESEKQR | |
| | LGGDVQSPNCPSEDVVITPESFGRDSSLTCLAGNVSACDAPILSSS | |
| | RSLDCRESGKNGPHVYQDLLLSLGTTNSTLPPPFSLQSGILTLNPVA | |
| | QGQPILTSLGSNQEEAYVTMSSFYQNQSDPKPENPACPWTVLPA | |
| | GDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 151 |
| 166).TPOR/MPLR(478- | PHVQKSVNNDMIVTDNNGAVKFPQLCKFCDVRFSTCDNQKSCM | |
| 582; N + 8).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLVLLILVLISLVTALHLVLGLSAVLGLLLLR | |
| | WQFPAHYRRLRHALWPSLPDLHRVLGQYLRDTAALSPPKATVSD | |
| | TCEEVEPSLLEILPKSSERTPLPLARDEVEGFLQDTFPQQLEESEKQR | |
| | LGGDVQSPNCPSEDVVITPESFGRDSSLTCLAGNVSACDAPILSSS | |
| | RSLDCRESGKNGPHVYQDLLLSLGTTNSTLPPPFSLQSGILTLNPVA | |
| | QGQPILTSLGSNQEEAYVTMSSFYQNQSDPKPENPACPWTVLPA | |
| | GDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 162 |
| 166).TPOR/MPLR(478- | PHVQKSVNNDMIVTDNNGAVKFPQLCKFCDVRFSTCDNQKSCM | |
| 582).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWISLVTALHLVLGLSAVLGLLLLRWQFPA | |
| | HYRRLRHALWPSLPDLHRVLGQYLRDTAALSPPKATVSDTCEEVE | |
| | PSLLEILPKSSERTPLPLLEARDEVEGFLQDTFPQQLEESEKQRLGG | |
| | DVQSPNCPSEDVVITPESFGRDSSLTCLAGNVSACDAPILSSSRSLD | |
| | CRESGKNGPHVYQDLLLSLGTTNSTLPPPFSLQSGILTLNPVAQGQ | |
| | PILTSLGSNQEEAYVTMSSFYQNQSRSDPKPENPACPWTVLPAG | |
| | DLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 163 |
| 166).TPOR/MPLR(478-582) | PHVQKSVNNDMIVTDNNGAVKFPQLCKFCDVRFSTCDNQKSCM | |
| S505N.W515K.IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWISLVTALHLVLGLNAVLGLLLLRKQFPAH | |
| | YRRLRHALWPSLPDLHRVLGQYLRDTAALSPPKATVSDTCEEVEPS | |
| | LLEILPKSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSRSDPKPENPACPWTVLPAGDLP | |
| | THDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 164 |
| 166).TPOR/MPLR(478-582) | PHVQKSVNNDMIVTDNNGAVKFPQLCKFCDVRFSTCDNQKSCM | |
| S505N.W515K.IL2Rbsmall | SNCSITSICEKPQEVCVAVWRKNDENITLETVCHDPKLPYHDFILED | |
| (393-433, 518-551) | AASPKCIMKEKKKPGETFFMCSCSSDECNDNIIFSEEYNTSNPDLLL | |
| Underlined indicates CD8SS | VIFQSDPTRVETATETAWISLVTALHLVLGLNAVLGLLLLRKQFPAH | |
| | YRRLRHALWPSLPDLHRVLGQYLRDTAALSPPKATVSDTCEEVEPS | |
| | LLEILPKSSERTPLPLLEDEGVAGAPTGSSPQPLQPLSGEDDAYCTF | |
| | PSRDDLLLFSPSGQGEFRALNARLPLNTDAYLSLQELQGQDPTHLV | |
| CD8SS-TGFbR2(1- | MALPVTALLLPLALLLHAARPMGRGLLRGLWPLHIVLWTRIASTIP | 165 |
| 166).TPOR/MPLR(478-582) | PHVQKSVNNDMIVTDNNGAVKFPQLCKFCDVRFSTCDNQKSCM | |
| S505N.W515K. | SNCSITSICEKPQEVCVAVWRKNDENITLETVCHDPKLPYHDFILED | |
| K553R.K573R.IL7Ra(316- | AASPKCIMKEKKKPGETFFMCSCSSDECNDNIIFSEEYNTSNPDLLL | |
| 459).IL12Rb2(775-825) | VIFQSDPTRVETATETAWISLVTALHLVLGLNAVLGLLLLRKQFPAH | |
| Underlined indicates CD8SS | YRRLRHALWPSLPDLHRVLGQYLRDTAALSPPRATVSDTCEEVEPS | |
| | LLEILPRSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSRSDPKPENPACPWTVLPAGDLP | |
| | THDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 166 |
| 166).TPOR/MPLR(478-582) | PHVQKSVNNDMIVTDNNGAVKFPQLCKFCDVRFSTCDNQKSCM | |
| S505N.W515K. | SNCSITSICEKPQEVCVAVWRKNDENITLETVCHDPKLPYHDFILED | |
| K553R.K573R. | AASPKCIMKEKKKPGETFFMCSCSSDECNDNIIFSEEYNTSNPDLLL | |
| IL2Rbsmall(393-433, | VIFQSDPTRVETATETAWISLVTALHLVLGLNAVLGLLLLRKQFPAH | |
| 518-551) | YRRLRHALWPSLPDLHRVLGQYLRDTAALSPPRATVSDTCEEVEPS | |
| Underlined indicates CD8SS | LLEILPRSSERTPLPLLEDEGVAGAPTGSSPQPLQPLSGEDDAYCTF | |
| | PSRDDLLLFSPSGQGEFRALNARLPLNTDAYLSLQELQGQDPTHLV | |
| TGFbR2(1- | MGRGLLRGLWPLHIVLWTRIASTIPPHVQKSVNNDMIVTDNNGA | 167 |
| 166).TPOR/MPLR(478-582) | VKFPQLCKFCDVRFSTCDNQKSCMSNCSITSICEKPQEVCVAVWR | |
| S505N.W515K.IL2Rbsmall | KNDENITLETVCHDPKLPYHDFILEDAASPKCIMKEKKKPGETFFM | |
| (393-433, 518-551) | CSCSSDECNDNIIFSEEYNTSNPDLLLVIFQSDPTRVETATETAWISL | |
| | VTALHLVLGLNAVLGLLLLRKQFPAHYRRLRHALWPSLPDLHRVLG | |
| | QYLRDTAALSPPKATVSDTCEEVEPSLLEILPKSSERTPLPLLEDEGV | |
| | AGAPTGSSPQPLQPLSGEDDAYCTFPSRDDLLLFSPSGQGEFRAL | |
| | NARLPLNTDAYLSLQELQGQDPTHLV | |
| TGFbR2(1- | MGRGLLRGLWPLHIVLWTRIASTIPPHVQKSVNNDMIVTDNNGA | 168 |
| 166).TPOR/MPLR(478-582) | VKFPQLCKFCDVRFSTCDNQKSCMSNCSITSICEKPQEVCVAVWR | |
| S505N.W515K. | KNDENITLETVCHDPKLPYHDFILEDAASPKCIMKEKKKPGETFFM | |
| K553R.K573R. | CSCSSDECNDNIIFSEEYNTSNPDLLLVIFQSDPTRVETATETAWISL | |
| IL2Rbsmall(393-433, | VTALHLVLGLNAVLGLLLLRKQFPAHYRRLRHALWPSLPDLHRVLG | |
| 518-551) | QYLRDTAALSPPRATVSDTCEEVEPSLLEILPRSSERTPLPLLEDEGV | |
| | AGAPTGSSPQPLQPLSGEDDAYCTFPSRDDLLLFSPSGQGEFRAL | |
| | NARLPLNTDAYLSLQELQGQDPTHLV | |
| CD8SS-TGFbR2(1- | MALPVTALLLPLALLLHAARPMGRGLLRGLWPLHIVLWTRIASTIP | 169 |
| 166).TPOR/MPLR(478-582) | PHVQKSVNNDMIVTDNNGAVKFPQLCKFCDVRFSTCDNQKSCM | |
| S505N.W515K.IL7Ra(316- | SNCSITSICEKPQEVCVAVWRKNDENITLETVCHDPKLPYHDFILED | |
| 459) | AASPKCIMKEKKKPGETFFMCSCSSDECNDNIIFSEEYNTSNPDLLL | |
| Underlined indicates CD8SS | VIFQSDPTRVETATETAWISLVTALHLVLGLNAVLGLLLLRKQFPAH | |
| | YRRLRHALWPSLPDLHRVLGQYLRDTAALSPPKATVSDTCEEVEPS | |
| | LLEILPKSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQ | |
| CD8SS-TGFbR2(1- | MALPVTALLLPLALLLHAARPMGRGLLRGLWPLHIVLWTRIASTIP | 170 |
| 166).TPOR/MPLR(478-582) | PHVQKSVNNDMIVTDNNGAVKFPQLCKFCDVRFSTCDNQKSCM | |
| S505N.W515K.K553R.K573R. | SNCSITSICEKPQEVCVAVWRKNDENITLETVCHDPKLPYHDFILED | |
| IL7Ra(316-459) | AASPKCIMKEKKKPGETFFMCSCSSDECNDNIIFSEEYNTSNPDLLL | |
| Underlined indicates CD8SS | VIFQSDPTRVETATETAWISLVTALHLVLGLNAVLGLLLLRKQFPAH | |
| | YRRLRHALWPSLPDLHRVLGQYLRDTAALSPPRATVSDTCEEVEPS | |
| | LLEILPRSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQ | |
| CD8SS-TGFbR2(1- | MALPVTALLLPLALLLHAARPMGRGLLRGLWPLHIVLWTRIASTIP | 171 |
| 166).TPOR/MPLR(478-582) | PHVQKSVNNDMIVTDNNGAVKFPQLCKFCDVRFSTCDNQKSCM | |
| S505N.W515K. | SNCSITSICEKPQEVCVAVWRKNDENITLETVCHDPKLPYHDFILED | |
| IL2Rbsmall(339-379, | AASPKCIMKEKKKPGETFFMCSCSSDECNDNIIFSEEYNTSNPDLLL | |
| 393-433, 518-551) | VIFQSDPTRVETATETAWISLVTALLLVLGLNAVLGLLLLRKQFPAH | |
| Underlined indicates CD8SS | YRRLRHALWPSLPDLHRVLGQYLRDTAALSPPKATVSDTCEEVEPS | |
| | LLEILPKSSERTPLPLLEQQDKVPEPASLSSNHSLTSCFTNQGYFFFH | |
| | LPDALEIEACQDEGVAGAPTGSSPQPLQPLSGEDDAYCTFPSRDD | |
| | LLLFSPSGQGEFRALNARLPLNTDAYLSLQELQGQDPTHLV | |
| CD8SS-TGFbR2(1- | MALPVTALLLPLALLLHAARPMGRGLLRGLWPLHIVLWTRIASTIP | 172 |
| 166).TPOR/MPLR(478- | PHVQKSVNNDMIVTDNNGAVKFPQLCKFCDVRFSTCDNQKSCM | |
| 582; N - 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETWISLVTALHLVLGLSAVLGLLLLRWQFPAH | |
| | YRRLRHALWPSLPDLHRVLGQYLRDTAALSPPKATVSDTCEEVEPS | |
| | LLEILPKSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSRSDPKPENPACPWTVLPAGDLP | |
| | THDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 173 |
| 166).TPOR/MPLR(478- | PHVQKSVNNDMIVTDNNGAVKFPQLCKFCDVRFSTCDNQKSCM | |
| 582; N - 2).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETISLVTALHLVLGLSAVLGLLLLRWQFPAHYR | |
| | RLRHALWPSLPDLHRVLGQYLRDTAALSPPKATVSDTCEEVEPSLL | |
| | EILPKSSERTPLPLLEARDEVEGFLQDTFPQQLEESEKQRLGGDVQ | |
| | SPNCPSEDVVITPESFGRDSSLTCLAGNVSACDAPILSSSRSLDCRE | |
| | SGKNGPHVYQDLLLSLGTTNSTLPPPFSLQSGILTLNPVAQGQPILT | |
| | SLGSNQEEAYVTMSSFYQNQSRSDPKPENPACPWTVLPAGDLPT | |
| | HDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 174 |
| 166).TPOR/MPLR(478- | PHVQKSVNNDMIVTDNNGAVKFPQLCKFCDVRFSTCDNQKSCM | |
| 582; N - 2 + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| | VIFQSDPTRVETATETLISLVTALHLVLGLSAVLGLLLLRWQFPAHY | |
| | RRLRHALWPSLPDLHRVLGQYLRDTAALSPPKATVSDTCEEVEPSL | |
| | LEILPKSSERTPLPLLEARDEVEGFLQDTFPQQLEESEKQRLGGDVQ | |
| | SPNCPSEDVVITPESFGRDSSLTCLAGNVSACDAPILSSSRSLDCRE | |
| | SGKNGPHVYQDLLLSLGTTNSTLPPPFSLQSGILTLNPVAQGQPILT | |
| | SLGSNQEEAYVTMSSFYQNQSRSDPKPENPACPWTVLPAGDLPT | |
| | HDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 175 |
| 166).TPOR/MPLR(478- | PHVQKSVNNDMIVTDNNGAVKFPQLCKFCDVRFSTCDNQKSCM | |
| 582; N - 3).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| | VIFQSDPTRVETATETSLVTALHLVLGLSAVLGLLLLRWQFPAHYRR | |
| | LRHALWPSLPDLHRVLGQYLRDTAALSPPKATVSDTCEEVEPSLLEI | |
| | LPKSSERTPLPLLEARDEVEGFLQDTFPQQLEESEKQRLGGDVQSP | |
| | NCPSEDVVITPESFGRDSSLTCLAGNVSACDAPILSSSRSLDCRESG | |
| | KNGPHVYQDLLLSLGTTNSTLPPPFSLQSGILTLNPVAQGQPILTSL | |
| | GSNQEEAYVTMSSFYQNQSRSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 176 |
| 166).TPOR/MPLR(478- | PHVQKSVNNDMIVTDNNGAVKFPQLCKFCDVRFSTCDNQKSCM | |
| 582; N - 4).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLVTALHLVLGLSAVLGLLLLRWQFPAHYRRL | |
| | RHALWPSLPDLHRVLGQYLRDTAALSPPKATVSDTCEEVEPSLLEIL | |
| | PKSSERTPLPLLEARDEVEGFLQDTFPQQLEESEKQRLGGDVQSP | |
| | NCPSEDVVITPESFGRDSSLTCLAGNVSACDAPILSSSRSLDCRESG | |
| | KNGPHVYQDLLLSLGTTNSTLPPPFSLQSGILTLNPVAQGQPILTSL | |
| | GSNQEEAYVTMSSFYQNQSRSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 177 |
| 166).TPOR/MPLR(478- | PHVQKSVNNDMIVTDNNGAVKFPQLCKFCDVRFSTCDNQKSCM | |
| 582; N - 4 + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETILVTALHLVLGLSAVLGLLLLRWQFPAHYRR | |
| | LRHALWPSLPDLHRVLGQYLRDTAALSPPKATVSDTCEEVEPSLLEI | |
| | LPKSSERTPLPLLEARDEVEGFLQDTFPQQLEESEKQRLGGDVQSP | |
| | NCPSEDVVITPESFGRDSSLTCLAGNVSACDAPILSSSRSLDCRESG | |
| | KNGPHVYQDLLLSLGTTNSTLPPPFSLQSGILTLNPVAQGQPILTSL | |
| | GSNQEEAYVTMSSFYQNQSRSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 178 |
| 166).TPOR/MPLR(478- | PHVQKSVNNDMIVTDNNGAVKFPQLCKFCDVRFSTCDNQKSCM | |
| 582; N - 5).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| | VIFQSDPTRVETATETVTALHLVLGLSAVLGLLLLRWQFPAHYRRL | |
| | RHALWPSLPDLHRVLGQYLRDTAALSPPKATVSDTCEEVEPSLLEIL | |
| | PKSSERTPLPLLEARDEVEGFLQDTFPQQLEESEKQRLGGDVQSP | |
| | NCPSEDVVITPESFGRDSSLTCLAGNVSACDAPILSSSRSLDCRESG | |
| | KNGPHVYQDLLLSLGTTNSTLPPPFSLQSGILTLNPVAQGQPILTSL | |
| | GSNQEEAYVTMSSFYQNQSRSDPKPENPACPWTVLPAGDLPTH | |
| | DGYLPSNIDDLPSHEAPLADSLEELEPQ | |
| CD8SS-TGFbR2(1- | MALPVTALLLPLALLLHAARPMGRGLLRGLWPLHIVLWTRIASTIP | 179 |
| 166).TPOR/MPLR(478- | PHVQKSVNNDMIVTDNNGAVKFPQLCKFCDVRFSTCDNQKSCM | |
| 582; N - 6).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETTALHLVLGLSAVLGLLLLRWQFPAHYRRLR | |
| | HALWPSLPDLHRVLGQYLRDTAALSPPKATVSDTCEEVEPSLLEILP | |
| | KSSERTPLPLLEARDEVEGFLQDTFPQQLEESEKQRLGGDVQSPN | |
| | CPSEDVVITPESFGRDSSLTCLAGNVSACDAPILSSSRSLDCRESGK | |
| | NGPHVYQDLLLSLGTTNSTLPPPFSLQSGILTLNPVAQGQPILTSLG | |
| | SNQEEAYVTMSSFYQNQSRSDPKPENPACPWTVLPAGDLPTHD | |
| | GYLPSNIDDLPSHEAPLADSLEELEPQ | |
| CD8SS-TGFbR2(1- | MALPVTALLLPLALLLHAARPMGRGLLRGLWPLHIVLWTRIASTIP | 180 |
| 166).TPOR/MPLR(478- | PHVQKSVNNDMIVTDNNGAVKFPQLCKFCDVRFSTCDNQKSCM | |
| 582; N - 7).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| | VIFQSDPTRVETATETALHLVLGLSAVLGLLLLRWQFPAHYRRLRH | |
| | ALWPSLPDLHRVLGQYLRDTAALSPPKATVSDTCEEVEPSLLEILPK | |
| | SSERTPLPLLEARDEVEGFLQDTFPQQLEESEKQRLGGDVQSPNC | |
| | PSEDVVITPESFGRDSSLTCLAGNVSACDAPILSSSRSLDCRESGKN | |
| | GPHVYQDLLLSLGTTNSTLPPPFSLQSGILTLNPVAQGQPILTSLGS | |
| | NQEEAYVTMSSFYQNQSRSDPKPENPACPWTVLPAGDLPTHDG | |
| | YLPSNIDDLPSHEAPLADSLEELEPQ | |
| CD8SS-TGFbR2(1- | MALPVTALLLPLALLLHAARPMGRGLLRGLWPLHIVLWTRIASTIP | 181 |
| 166).TPOR/MPLR(478- | PHVQKSVNNDMIVTDNNGAVKFPQLCKFCDVRFSTCDNQKSCM | |
| 582; N - 8).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLHLVLGLSAVLGLLLLRWQFPAHYRRLRHA | |
| | LWPSLPDLHRVLGQYLRDTAALSPPKATVSDTCEEVEPSLLEILPKS | |
| | SERTPLPLLEARDEVEGFLQDTFPQQLEESEKQRLGGDVQSPNCP | |
| | SEDVVITPESFGRDSSLTCLAGNVSACDAPILSSSRSLDCRESGKNG | |
| | PHVYQDLLLSLGTTNSTLPPPFSLQSGILTLNPVAQGQPILTSLGSN | |
| | QEEAYVTMSSFYQNQSRSDPKPENPACPWTVLPAGDLPTHDGYL | |
| | PSNIDDLPSHEAPLADSLEELEPQ | |
| CD8SS-TGFbR2(1- | MALPVTALLLPLALLLHAARPMGRGLLRGLWPLHIVLWTRIASTIP | 182 |
| 166).TPOR/MPLR(478- | PHVQKSVNNDMIVTDNNGAVKFPQLCKFCDVRFSTCDNQKSCM | |
| 582; N - 9).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETHLVLGLSAVLGLLLLRWQFPAHYRRLRHAL | |
| | WPSLPDLHRVLGQYLRDTAALSPPKATVSDTCEEVEPSLLEILPKSS | |
| | ERTPLPLLEARDEVEGFLQDTFPQQLEESEKQRLGGDVQSPNCPS | |
| | EDVVITPESFGRDSSLTCLAGNVSACDAPILSSSRSLDCRESGKNGP | |
| | HVYQDLLLSLGTTNSTLPPPFSLQSGILTLNPVAQGQPILTSLGSNQ | |
| | EEAYVTMSSFYQNQSRSDPKPENPACPWTVLPAGDLPTHDGYLP | |
| | SNIDDLPSHEAPLADSLEELEPQ | |
| CD8SS-TGFbR2(1- | MALPVTALLLPLALLLHAARPMGRGLLRGLWPLHIVLWTRIASTIP | 183 |
| 166).TPOR/MPLR(478- | PHVQKSVNNDMIVTDNNGAVKFPQLCKFCDVRFSTCDNQKSCM | |
| 582; N - 10).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLVLGLSAVLGLLLLRWQFPAHYRRLRHALW | |
| | PSLPDLHRVLGQYLRDTAALSPPKATVSDTCEEVEPSLLEILPKSSER | |
| | TPLPLLEARDEVEGFLQDTFPQQLEESEKQRLGGDVQSPNCPSED | |
| | VVITPESFGRDSSLTCLAGNVSACDAPILSSSRSLDCRESGKNGPHV | |
| | YQDLLLSLGTTNSTLPPPFSLQSGILTLNPVAQGQPILTSLGSNQEE | |
| | AYVTMSSFYQNQSRSDPKPENPACPWTVLPAGDLPTHDGYLPSN | |
| | IDDLPSHEAPLADSLEELEPQ | |
| CD8SS-TGFbR2(1- | MALPVTALLLPLALLLHAARPMGRGLLRGLWPLHIVLWTRIASTIP | 184 |
| 166).TPOR/MPLR(478- | PHVQKSVNNDMIVTDNNGAVKFPQLCKFCDVRFSTCDNQKSCM | |
| 582; N - 11).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETVLGLSAVLGLLLLRWQFPAHYRRLRHALW | |
| | PSLPDLHRVLGQYLRDTAALSPPKATVSDTCEEVEPSLLEILPKSSER | |
| | TPLPLLEARDEVEGFLQDTFPQQLEESEKQRLGGDVQSPNCPSED | |
| | VVITPESFGRDSSLTCLAGNVSACDAPILSSSRSLDCRESGKNGPHV | |
| | YQDLLLSLGTTNSTLPPPFSLQSGILTLNPVAQGQPILTSLGSNQEE | |
| | AYVTMSSFYQNQSRSDPKPENPACPWTVLPAGDLPTHDGYLPSN | |
| | IDDLPSHEAPLADSLEELEPQ | |
| CD8SS-TGFbR2(1- | MALPVTALLLPLALLLHAARPMGRGLLRGLWPLHIVLWTRIASTIP | 185 |
| 166).TPOR/MPLR(478- | PHVQKSVNNDMIVTDNNGAVKFPQLCKFCDVRFSTCDNQKSCM | |
| 582; N - 12).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLGLSAVLGLLLLRWQFPAHYRRLRHALWPS | |
| | LPDLHRVLGQYLRDTAALSPPKATVSDTCEEVEPSLLEILPKSSERTP | |
| | LPLLEARDEVEGFLQDTFPQQLEESEKQRLGGDVQSPNCPSEDVV | |
| | ITPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSRSDPKPENPACPWTVLPAGDLPTHDGYLPSNID | |
| | DLPSHEAPLADSLEELEPQ | |
| CD8SS-TGFbR2(1- | MALPVTALLLPLALLLHAARPMGRGLLRGLWPLHIVLWTRIASTIP | 186 |
| 166).TPOR/MPLR(478- | PHVQKSVNNDMIVTDNNGAVKFPQLCKFCDVRFSTCDNQKSCM | |
| 582; N - 13).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETGLSAVLGLLLLRWQFPAHYRRLRHALWPSL | |
| | PDLHRVLGQYLRDTAALSPPKATVSDTCEEVEPSLLEILPKSSERTPL | |
| | PLLEARDEVEGFLQDTFPQQLEESEKQRLGGDVQSPNCPSEDVVI | |
| | TPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSRSDPKPENPACPWTVLPAGDLPTHDGYLPSNID | |
| | DLPSHEAPLADSLEELEPQ | |
| CD8SS-TGFbR2(1- | MALPVTALLLPLALLLHAARPMGRGLLRGLWPLHIVLWTRIASTIP | 187 |
| 166).TPOR/MPLR(478- | PHVQKSVNNDMIVTDNNGAVKFPQLCKFCDVRFSTCDNQKSCM | |
| 582; N - 14).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLSAVLGLLLLRWQFPAHYRRLRHALWPSLP | |
| | DLHRVLGQYLRDTAALSPPKATVSDTCEEVEPSLLEILPKSSERTPLP | |
| | LLEARDEVEGFLQDTFPQQLEESEKQRLGGDVQSPNCPSEDVVIT | |
| | PESFGRDSSLTCLAGNVSACDAPILSSSRSLDCRESGKNGPHVYQD | |
| | LLLSLGTTNSTLPPPFSLQSGILTLNPVAQGQPILTSLGSNQEEAYVT | |
| | MSSFYQNQSRSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 188 |
| 166).TPOR/MPLR(478- | PHVQKSVNNDMIVTDNNGAVKFPQLCKFCDVRFSTCDNQKSCM | |
| 582; N - 15).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETSAVLGLLLLRWQFPAHYRRLRHALWPSLPD | |
| | LHRVLGQYLRDTAALSPPKATVSDTCEEVEPSLLEILPKSSERTPLPL | |
| | LEARDEVEGFLQDTFPQQLEESEKQRLGGDVQSPNCPSEDVVITP | |
| | ESFGRDSSLTCLAGNVSACDAPILSSSRSLDCRESGKNGPHVYQDL | |
| | LLSLGTTNSTLPPPFSLQSGILTLNPVAQGQPILTSLGSNQEEAYVT | |
| | MSSFYQNQSRSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 189 |
| 166).TPOR/MPLR(478- | PHVQKSVNNDMIVTDNNGAVKFPQLCKFCDVRFSTCDNQKSCM | |
| 582; N - 16).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAVLGLLLLRWQFPAHYRRLRHALWPSLPDL | |
| | HRVLGQYLRDTAALSPPKATVSDTCEEVEPSLLEILPKSSERTPLPLL | |
| | EARDEVEGFLQDTFPQQLEESEKQRLGGDVQSPNCPSEDVVITPE | |
| | SFGRDSSLTCLAGNVSACDAPILSSSRSLDCRESGKNGPHVYQDLL | |
| | LSLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 190 |
| 166).TPOR/MPLR(478- | PHVQKSVNNDMIVTDNNGAVKFPQLCKFCDVRFSTCDNQKSCM | |
| 582; N - 17).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETVLGLLLLRWQFPAHYRRLRHALWPSLPDLH | |
| | RVLGQYLRDTAALSPPKATVSDTCEEVEPSLLEILPKSSERTPLPLLE | |
| | ARDEVEGFLQDTFPQQLEESEKQRLGGDVQSPNCPSEDVVITPES | |
| | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 191 |
| 166).TPOR/MPLR(478- | PHVQKSVNNDMIVTDNNGAVKFPQLCKFCDVRFSTCDNQKSCM | |
| 582; N - 18).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETLGLLLLRWQFPAHYRRLRHALWPSLPDLHR | |
| | VLGQYLRDTAALSPPKATVSDTCEEVEPSLLEILPKSSERTPLPLLEA | |
| | RDEVEGFLQDTFPQQLEESEKQRLGGDVQSPNCPSEDVVITPESF | |
| | GRDSSLTCLAGNVSACDAPILSSSRSLDCRESGKNGPHVYQDLLLS | |
| | LGTTNSTLPPPFSLQSGILTLNPVAQGQPILTSLGSNQEEAYVTMS | |
| | SFYQNQSRSDPKPENPACPWTVLPAGDLPTHDGYLPSNIDDLPSH | |
| | EAPLADSLEELEPQ | |
| CD8SS-TGFbR2(1- | MALPVTALLLPLALLLHAARPMGRGLLRGLWPLHIVLWTRIASTIP | 192 |
| 166).TPOR/MPLR(478- | PHVQKSVNNDMIVTDNNGAVKFPQLCKFCDVRFSTCDNQKSCM | |
| 582; N + 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLISLVTALHLVLGLSAVLGLLLLRWQFPA | |
| | HYRRLRHALWPSLPDLHRVLGQYLRDTAALSPPKATVSDTCEEVE | |
| | PSLLEILPKSSERTPLPLLEARDEVEGFLQDTFPQQLEESEKQRLGG | |
| | DVQSPNCPSEDVVITPESFGRDSSLTCLAGNVSACDAPILSSSRSLD | |
| | CRESGKNGPHVYQDLLLSLGTTNSTLPPPFSLQSGILTLNPVAQGQ | |
| | PILTSLGSNQEEAYVTMSSFYQNQSRSDPKPENPACPWTVLPAGD | |
| | LPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 193 |
| 166).TPOR/MPLR(478- | PHVQKSVNNDMIVTDNNGAVKFPQLCKFCDVRFSTCDNQKSCM | |
| 582; N + 2).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWVLISLVTALHLVLGLSAVLGLLLLRWQFP | |
| | AHYRRLRHALWPSLPDLHRVLGQYLRDTAALSPPKATVSDTCEEV | |
| | EPSLLEILPKSSERTPLPLLEARDEVEGFLQDTFPQQLEESEKQRLGG | |
| | DVQSPNCPSEDVVITPESFGRDSSLTCLAGNVSACDAPILSSSRSLD | |
| | CRESGKNGPHVYQDLLLSLGTTNSTLPPPFSLQSGILTLNPVAQGQ | |
| | PILTSLGSNQEEAYVTMSSFYQNQSRSDPKPENPACPWTVLPAGD | |
| | LPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 194 |
| 166).TPOR/MPLR(478- | PHVQKSVNNDMIVTDNNGAVKFPQLCKFCDVRFSTCDNQKSCM | |
| 582; N + 3).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLVLISLVTALHLVLGLSAVLGLLLLRWQF | |
| | PAHYRRLRHALWPSLPDLHRVLGQYLRDTAALSPPKATVSDTCEE | |
| | VEPSLLEILPKSSERTPLPLLEARDEVEGFLQDTFPQQLEESEKQRLG | |
| | GDVQSPNCPSEDVVITPESFGRDSSLTCLAGNVSACDAPILSSSRSL | |
| | DCRESGKNGPHVYQDLLLSLGTTNSTLPPPFSLQSGILTLNPVAQG | |
| | QPILTSLGSNQEEAYVTMSSFYQNQSRSDPKPENPACPWTVLPA | |
| | GDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 195 |
| 166).TPOR/MPLR(478- | PHVQKSVNNDMIVTDNNGAVKFPQLCKFCDVRFSTCDNQKSCM | |
| 582; N + 4).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWILVLISLVTALHLVLGLSAVLGLLLLRWQ | |
| | FPAHYRRLRHALWPSLPDLHRVLGQYLRDTAALSPPKATVSDTCE | |
| | EVEPSLLEILPKSSERTPLPLLEARDEVEGFLQDTFPQQLEESEKQRL | |
| | GGDVQSPNCPSEDVVITPESFGRDSSLTCLAGNVSACDAPILSSSR | |
| | SLDCRESGKNGPHVYQDLLLSLGTTNSTLPPPFSLQSGILTLNPVA | |
| | QGQPILTSLGSNQEEAYVTMSSFYQNQSRSDPKPENPACPWTVL | |
| | PAGDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 196 |
| 166).TPOR/MPLR(478- | PHVQKSVNNDMIVTDNNGAVKFPQLCKFCDVRFSTCDNQKSCM | |
| 582; N + 5).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLILVLISLVTALHLVLGLSAVLGLLLLRW | |
| | QFPAHYRRLRHALWPSLPDLHRVLGQYLRDTAALSPPKATVSDTC | |
| | EEVEPSLLEILPKSSERTPLPLLEARDEVEGFLQDTFPQQLEESEKQR | |
| | LGGDVQSPNCPSEDVVITPESFGRDSSLTCLAGNVSACDAPILSSS | |
| | RSLDCRESGKNGPHVYQDLLLSLGTTNSTLPPPFSLQSGILTLNPVA | |
| | QGQPILTSLGSNQEEAYVTMSSFYQNQSRSDPKPENPACPWTVL | |
| | PAGDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 197 |
| 166).TPOR/MPLR(478- | PHVQKSVNNDMIVTDNNGAVKFPQLCKFCDVRFSTCDNQKSCM | |
| 582; N + 6).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLLILVLISLVTALHLVLGLSAVLGLLLLRW | |
| | QFPAHYRRLRHALWPSLPDLHRVLGQYLRDTAALSPPKAWSDTC | |
| | EEVEPSLLEILPKSSERTPLPLLEARDEVEGFLQDTFPQQLEESEKQR | |
| | LGGDVQSPNCPSEDVVITPESFGRDSSLTCLAGNVSACDAPILSSS | |
| | RSLDCRESGKNGPHVYQDLLLSLGTTNSTLPPPFSLQSGILTLNPVA | |
| | QGQPILTSLGSNQEEAYVTMSSFYQNQSRSDPKPENPACPWTVL | |
| | PAGDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 198 |
| 166).TPOR/MPLR(478- | PHVQKSVNNDMIVTDNNGAVKFPQLCKFCDVRFSTCDNQKSCM | |
| 582; N + 7).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWVLLILVLISLVTALHLVLGLSAVLGLLLLR | |
| | WQFPAHYRRLRHALWPSLPDLHRVLGQYLRDTAALSPPKATVSD | |
| | TCEEVEPSLLEILPKSSERTPLPLLEARDEVEGFLQDTFPQQLEESEK | |
| | QRLGGDVQSPNCPSEDVVITPESFGRDSSLTCLAGNVSACDAPILS | |
| | SSRSLDCRESGKNGPHVYQDLLLSLGTTNSTLPPPFSLQSGILTLNP | |
| | VAQGQPILTSLGSNQEEAYVTMSSFYQNQSRSDPKPENPACPWT | |
| | VLPAGDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 199 |
| 166).TPOR/MPLR(478- | PHVQKSVNNDMIVTDNNGAVKFPQLCKFCDVRFSTCDNQKSCM | |
| 582; N + 8).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATETAWLVLLILVLISLVTALHLVLGLSAVLGLLLLR | |
| | WQFPAHYRRLRHALWPSLPDLHRVLGQYLRDTAALSPPKATVSD | |
| | TCEEVEPSLLEILPKSSERTPLPLLEARDEVEGFLQDTFPQQLEESEK | |
| | QRLGGDVQSPNCPSEDVVITPESFGRDSSLTCLAGNVSACDAPILS | |
| | SSRSLDCRESGKNGPHVYQDLLLSLGTTNSTLPPPFSLQSGILTLNP | |
| | VAQGQPILTSLGSNQEEAYVTMSSFYQNQSRSDPKPENPACPWT | |
| | VLPAGDLPTHDGYLPSNIDDLPSHEAPLADSLEELEPQ | |
| CD8SS-TGFbR2(1- | MALPVTALLLPLALLLHAARPMGRGLLRGLWPLHIVLWTRIASTIP | 200 |
| 166).TPOR/MPLR(478- | PHVQKSVNNDMIVTDNNGAVKFPQLCKFCDVRFSTCDNQKSCM | |
| 582; N - 9 - 1).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATEHLVLGLSAVLGLLLLRWQFPAHYRRLRHAL | |
| | WPSLPDLHRVLGQYLRDTAALSPPKATVSDTCEEVEPSLLEILPKSS | |
| | ERTPLPLLEARDEVEGFLQDTFPQQLEESEKQRLGGDVQSPNCPS | |
| | EDVVITPESFGRDSSLTCLAGNVSACDAPILSSSRSLDCRESGKNGP | |
| | HVYQDLLLSLGTTNSTLPPPFSLQSGILTLNPVAQGQPILTSLGSNQ | |
| | EEAYVTMSSFYQNQSRSDPKPENPACPWTVLPAGDLPTHDGYLP | |
| | SNIDDLPSHEAPLADSLEELEPQ | |
| CD8SS-TGFbR2(1- | MALPVTALLLPLALLLHAARPMGRGLLRGLWPLHIVLWTRIASTIP | 201 |
| 166).TPOR/MPLR(478- | PHVQKSVNNDMIVTDNNGAVKFPQLCKFCDVRFSTCDNQKSCM | |
| 582; N - 9 - 2).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETATHLVLGLSAVLGLLLLRWQFPAHYRRLRHALW | |
| | PSLPDLHRVLGQYLRDTAALSPPKATVSDTCEEVEPSLLEILPKSSER | |
| | TPLPLLEARDEVEGFLQDTFPQQLEESEKQRLGGDVQSPNCPSED | |
| | VVITPESFGRDSSLTCLAGNVSACDAPILSSSRSLDCRESGKNGPHV | |
| | YQDLLLSLGTTNSTLPPPFSLQSGILTLNPVAQGQPILTSLGSNQEE | |
| | AYVTMSSFYQNQSRSDPKPENPACPWTVLPAGDLPTHDGYLPSN | |
| | IDDLPSHEAPLADSLEELEPQ | |
| CD8SS-TGFbR2(1- | MALPVTALLLPLALLLHAARPMGRGLLRGLWPLHIVLWTRIASTIP | 202 |
| 166).TPOR/MPLR(478- | PHVQKSVNNDMIVTDNNGAVKFPQLCKFCDVRFSTCDNQKSCM | |
| 582; N - 9 - 3).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETAHLVLGLSAVLGLLLLRWQFPAHYRRLRHALWP | |
| | SLPDLHRVLGQYLRDTAALSPPKATVSDTCEEVEPSLLEILPKSSERT | |
| | PLPLLEARDEVEGFLQDTFPQQLEESEKQRLGGDVQSPNCPSEDV | |
| | VITPESFGRDSSLTCLAGNVSACDAPILSSSRSLDCRESGKNGPHVY | |
| | QDLLLSLGTTNSTLPPPFSLQSGILTLNPVAQGQPILTSLGSNQEEA | |
| | YVTMSSFYQNQSRSDPKPENPACPWTVLPAGDLPTHDGYLPSNI | |
| | DDLPSHEAPLADSLEELEPQ | |
| CD8SS-TGFbR2(1- | MALPVTALLLPLALLLHAARPMGRGLLRGLWPLHIVLWTRIASTIP | 203 |
| 166).TPOR/MPLR(478- | PHVQKSVNNDMIVTDNNGAVKFPQLCKFCDVRFSTCDNQKSCM | |
| 582; N - 9 - 4).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVETHLVLGLSAVLGLLLLRWQFPAHYRRLRHALWPSL | |
| | PDLHRVLGQYLRDTAALSPPKATVSDTCEEVEPSLLEILPKSSERTPL | |
| | PLLEARDEVEGFLQDTFPQQLEESEKQRLGGDVQSPNCPSEDVVI | |
| | TPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSRSDPKPENPACPWTVLPAGDLPTHDGYLPSNID | |
| | DLPSHEAPLADSLEELEPQ | |
| CD8SS-TGFbR2(1- | MALPVTALLLPLALLLHAARPMGRGLLRGLWPLHIVLWTRIASTIP | 204 |
| 166).TPOR/MPLR(478- | PHVQKSVNNDMIVTDNNGAVKFPQLCKFCDVRFSTCDNQKSCM | |
| 582; N - 9 - 5).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVEHLVLGLSAVLGLLLLRWQFPAHYRRLRHALWPSLP | |
| | DLHRVLGQYLRDTAALSPPKATVSDTCEEVEPSLLEILPKSSERTPLP | |
| | LLEARDEVEGFLQDTFPQQLEESEKQRLGGDVQSPNCPSEDVVIT | |
| | PESFGRDSSLTCLAGNVSACDAPILSSSRSLDCRESGKNGPHVYQD | |
| | LLLSLGTTNSTLPPPFSLQSGILTLNPVAQGQPILTSLGSNQEEAYVT | |
| | MSSFYQNQSRSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 205 |
| 166).TPOR/MPLR(478- | PHVQKSVNNDMIVTDNNGAVKFPQLCKFCDVRFSTCDNQKSCM | |
| 582; N - 9 - 6).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRVHLVLGLSAVLGLLLLRWQFPAHYRRLRHALWPSLP | |
| | DLHRVLGQYLRDTAALSPPKATVSDTCEEVEPSLLEILPKSSERTPLP | |
| | LLEARDEVEGFLQDTFPQQLEESEKQRLGGDVQSPNCPSEDVVIT | |
| | PESFGRDSSLTCLAGNVSACDAPILSSSRSLDCRESGKNGPHVYQD | |
| | LLLSLGTTNSTLPPPFSLQSGILTLNPVAQGQPILTSLGSNQEEAYVT | |
| | MSSFYQNQSRSDPKPENPACPWTVLPAGDLPTHDGYLPSNIDDL | |
| | PSHEAPLADSLEELEPQ | |
| CD8SS-TGFbR2(1- | MALPVTALLLPLALLLHAARPMGRGLLRGLWPLHIVLWTRIASTIP | 206 |
| 166).TPOR/MPLR(478- | PHVQKSVNNDMIVTDNNGAVKFPQLCKFCDVRFSTCDNQKSCM | |
| 582; N - 9 - 7).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTRHLVLGLSAVLGLLLLRWQFPAHYRRLRHALWPSLPDL | |
| | HRVLGQYLRDTAALSPPKATVSDTCEEVEPSLLEILPKSSERTPLPLL | |
| | EARDEVEGFLQDTFPQQLEESEKQRLGGDVQSPNCPSEDVVITPE | |
| | SFGRDSSLTCLAGNVSACDAPILSSSRSLDCRESGKNGPHVYQDLL | |
| | LSLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 207 |
| 166).TPOR/MPLR(478- | PHVQKSVNNDMIVTDNNGAVKFPQLCKFCDVRFSTCDNQKSCM | |
| 582; N - 9 - 8).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPTHLVLGLSAVLGLLLLRWQFPAHYRRLRHALWPSLPDLH | |
| | RVLGQYLRDTAALSPPKATVSDTCEEVEPSLLEILPKSSERTPLPLLE | |
| | ARDEVEGFLQDTFPQQLEESEKQRLGGDVQSPNCPSEDVVITPES | |
| | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 208 |
| 166).TPOR/MPLR(478- | PHVQKSVNNDMIVTDNNGAVKFPQLCKFCDVRFSTCDNQKSCM | |
| 582; N - 9 - 9).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDPHLVLGLSAVLGLLLLRWQFPAHYRRLRHALWPSLPDLH | |
| | RVLGQYLRDTAALSPPKATVSDTCEEVEPSLLEILPKSSERTPLPLLE | |
| | ARDEVEGFLQDTFPQQLEESEKQRLGGDVQSPNCPSEDVVITPES | |
| | FGRDSSLTCLAGNVSACDAPILSSSRSLDCRESGKNGPHVYQDLLL | |
| | SLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS-TGFbR2(1- | MALPVTALLLPLALLLHAARPMGRGLLRGLWPLHIVLWTRIASTIP | 209 |
| 166).TPOR/MPLR(478- | PHVQKSVNNDMIVTDNNGAVKFPQLCKFCDVRFSTCDNQKSCM | |
| 582; N - 9 - 10).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSDHLVLGLSAVLGLLLLRWQFPAHYRRLRHALWPSLPDLHR | |
| | VLGQYLRDTAALSPPKATVSDTCEEVEPSLLEILPKSSERTPLPLLEA | |
| | RDEVEGFLQDTFPQQLEESEKQRLGGDVQSPNCPSEDVVITPESF | |
| | GRDSSLTCLAGNVSACDAPILSSSRSLDCRESGKNGPHVYQDLLLS | |
| | LGTTNSTLPPPFSLQSGILTLNPVAQGQPILTSLGSNQEEAYVTMS | |
| | SFYQNQSRSDPKPENPACPWTVLPAGDLPTHDGYLPSNIDDLPSH | |
| | EAPLADSLEELEPQ | |
| CD8SS-TGFbR2(1- | MALPVTALLLPLALLLHAARPMGRGLLRGLWPLHIVLWTRIASTIP | 210 |
| 166).TPOR/MPLR(478- | PHVQKSVNNDMIVTDNNGAVKFPQLCKFCDVRFSTCDNQKSCM | |
| 582; N - 9 - 11).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQSHLVLGLSAVLGLLLLRWQFPAHYRRLRHALWPSLPDLHRVL | |
| | GQYLRDTAALSPPKATVSDTCEEVEPSLLEILPKSSERTPLPLLEARD | |
| | EVEGFLQDTFPQQLEESEKQRLGGDVQSPNCPSEDVVITPESFGR | |
| | DSSLTCLAGNVSACDAPILSSSRSLDCRESGKNGPHVYQDLLLSLGT | |
| | TNSTLPPPFSLQSGILTLNPVAQGQPILTSLGSNQEEAYVTMSSFY | |
| | QNQSRSDPKPENPACPWTVLPAGDLPTHDGYLPSNIDDLPSHEA | |
| | PLADSLEELEPQ | |
| CD8SS-TGFbR2(1- | MALPVTALLLPLALLLHAARPMGRGLLRGLWPLHIVLWTRIASTIP | 211 |
| 166).TPOR/MPLR(478- | PHVQKSVNNDMIVTDNNGAVKFPQLCKFCDVRFSTCDNQKSCM | |
| 582; N - 9 - 12).IL7Ra(316- | SNCSITSICEKPQEVCVAVWRKNDENITLETVCHDPKLPYHDFILED | |
| 459).IL12Rb2(775-825) | AASPKCIMKEKKKPGETFFMCSCSSDECNDNIIFSEEYNTSNPDLLL | |
| Underlined indicates CD8SS | VIFQHLVLGLSAVLGLLLLRWQFPAHYRRLRHALWPSLPDLHRVL | |
| | GQYLRDTAALSPPKATVSDTCEEVEPSLLEILPKSSERTPLPLLEARD | |
| | EVEGFLQDTFPQQLEESEKQRLGGDVQSPNCPSEDVVITPESFGR | |
| | DSSLTCLAGNVSACDAPILSSSRSLDCRESGKNGPHVYQDLLLSLGT | |
| | TNSTLPPPFSLQSGILTLNPVAQGQPILTSLGSNQEEAYVTMSSFY | |
| | QNQSRSDPKPENPACPWTVLPAGDLPTHDGYLPSNIDDLPSHEA | |
| | PLADSLEELEPQ | |
| CD8SS- | MALPVTALLLPLALLLHAARPQLCKFCDVRFSTCDNQKSCMSNCSI | 212 |
| TGFbR2▴N25.TPOR/MPLR(478- | TSICEKPQEVCVAVWRKNDENITLETVCHDPKLPYHDFILEDAASP | |
| 582; N - 9).IL7Ra(316- | KCIMKEKKKPGETFFMCSCSSDECNDNIIFSEEYNTSNPDLLLVIFQ | |
| 459).IL12Rb2(775-825) | SDPTRVETATETHLVLGLSAVLGLLLLRWQFPAHYRRLRHALWPSL | |
| Underlined indicates CD8SS | PDLHRVLGQYLRDTAALSPPKATVSDTCEEVEPSLLEILPKSSERTPL | |
| | PLLEARDEVEGFLQDTFPQQLEESEKQRLGGDVQSPNCPSEDVVI | |
| | TPESFGRDSSLTCLAGNVSACDAPILSSSRSLDCRESGKNGPHVYQ | |
| | DLLLSLGTTNSTLPPPFSLQSGILTLNPVAQGQPILTSLGSNQEEAY | |
| | VTMSSFYQNQSRSDPKPENPACPWTVLPAGDLPTHDGYLPSNID | |
| | DLPSHEAPLADSLEELEPQ | |
| CD8SS- | MALPVTALLLPLALLLHAARPQLCKFCDVRFSTCDNQKSCMSNCSI | 213 |
| TGFbR2▴N25.TPOR/MPLR(478- | TSICEKPQEVCVAVWRKNDENITLETVCHDPKLPYHDFILEDAASP | |
| 582; N + 4).IL7Ra(316- | KCIMKEKKKPGETFFMCSCSSDECNDNIIFSEEYNTSNPDLLLVIFQ | |
| 459).IL12Rb2(775-825) | SDPTRVETATETAWILVLISLVTALHLVLGLSAVLGLLLLRWQFPAH | |
| Underlined indicates CD8SS | YRRLRHALWPSLPDLHRVLGQYLRDTAALSPPKATVSDTCEEVEPS | |
| | LLEILPKSSERTPLPLLEARDEVEGFLQDTFPQQLEESEKQRLGGDV | |
| | QSPNCPSEDVVITPESFGRDSSLTCLAGNVSACDAPILSSSRSLDCR | |
| | ESGKNGPHVYQDLLLSLGTTNSTLPPPFSLQSGILTLNPVAQGQPIL | |
| | TSLGSNQEEAYVTMSSFYQNQSRSDPKPENPACPWTVLPAGDLP | |
| | THDGYLPSNIDDLPSHEAPLADSLEELEPQ | |
| CD8SS- | MALPVTALLLPLALLLHAARPQLCKFCDVRFSTCDNQKSCMSNCSI | 214 |
| TGFbR2▴N25.TPOR/MPLR(478- | TSICEKPQEVCVAVWRKNDENITLETVCHDPKLPYHDFILEDAASP | |
| 582; N - 9 - 4).IL7Ra(316- | KCIMKEKKKPGETFFMCSCSSDECNDNIIFSEEYNTSNPDLLLVIFQ | |
| 459).IL12Rb2(775-825) | SDPTRVETHLVLGLSAVLGLLLLRWQFPAHYRRLRHALWPSLPDL | |
| Underlined indicates CD8SS | HRVLGQYLRDTAALSPPKATVSDTCEEVEPSLLEILPKSSERTPLPLL | |
| | EARDEVEGFLQDTFPQQLEESEKQRLGGDVQSPNCPSEDVVITPE | |
| | SFGRDSSLTCLAGNVSACDAPILSSSRSLDCRESGKNGPHVYQDLL | |
| | LSLGTTNSTLPPPFSLQSGILTLNPVAQGQPILTSLGSNQEEAYVTM | |
| | SSFYQNQSRSDPKPENPACPWTVLPAGDLPTHDGYLPSNIDDLPS | |
| | HEAPLADSLEELEPQ | |
| CD8SS- | MALPVTALLLPLALLLHAARPQLCKFCDVRFSTCDNQKSCMSNCSI | 215 |
| TGFbR2▴N25.TPOR/MPLR(478- | TSICEKPQEVCVAVWRKNDENITLETVCHDPKLPYHDFILEDAASP | |
| 582; N - 9 - 9).IL7Ra(316- | KCIMKEKKKPGETFFMCSCSSDECNDNIIFSEEYNTSNPDLLLVIFQ | |
| 459).IL12Rb2(775-825) | SDPHLVLGLSAVLGLLLLRWQFPAHYRRLRHALWPSLPDLHRVLG | |
| Underlined indicates CD8SS | QYLRDTAALSPPKATVSDTCEEVEPSLLEILPKSSERTPLPLLEARDE | |
| | VEGFLQDTFPQQLEESEKQRLGGDVQSPNCPSEDVVITPESFGRD | |
| | SSLTCLAGNVSACDAPILSSSRSLDCRESGKNGPHVYQDLLLSLGTT | |
| | NSTLPPPFSLQSGILTLNPVAQGQPILTSLGSNQEEAYVTMSSFYQ | |
| | NQSRSDPKPENPACPWTVLPAGDLPTHDGYLPSNIDDLPSHEAPL | |
| | ADSLEELEPQ | |
| CD8SS- | MALPVTALLLPLALLLHAARPQLCKFCDVRFSTCDNQKSCMSNCSI | 216 |
| TGFbR2▴N25.TPOR/MPLR | TSICEKPQEVCVAVWRKNDENITLETVCHDPKLPYHDFILEDAASP | |
| (478-582; N - 9 - 10).IL7Ra | KCIMKEKKKPGETFFMCSCSSDECNDNIIFSEEYNTSNPDLLLVIFQ | |
| (316-459).IL12Rb2(775-825) | SDHLVLGLSAVLGLLLLRWQFPAHYRRLRHALWPSLPDLHRVLGQ | |
| Underlined indicates CD8SS | YLRDTAALSPPKATVSDTCEEVEPSLLEILPKSSERTPLPLLEARDEV | |
| | EGFLQDTFPQQLEESEKQRLGGDVQSPNCPSEDVVITPESFGRDS | |
| | SLTCLAGNVSACDAPILSSSRSLDCRESGKNGPHVYQDLLLSLGTT | |
| | NSTLPPPFSLQSGILTLNPVAQGQPILTSLGSNQEEAYVTMSSFYQ | |
| | NQSRSDPKPENPACPWTVLPAGDLPTHDGYLPSNIDDLPSHEAPL | |
| | ADSLEELEPQ | |
| CD8SS-TPOR/MPLR(478- | MALPVTALLLPLALLLHAARPSDPTRVETATETAWISLVTALLLVLG | 272 |
| 582; H499L, S505N, W515K, | LNAVLGLLLLRKQFPAHYRRLRHALWPSLPDLHRVLGQYLRDTAA | |
| K553R, K573R).IL2Rb(339- | LSPPRATVSDTCEEVEPSLLEILPRSSERTPLPLLEQQDKVPEPASLS | |
| 379, 393-433, 518-551) | SNHSLTSCFTNQGYFFFHLPDALEIEACQDEGVAGAPTGSSPQPL | |
| Underlined indicates CD8SS | QPLSGEDDAYCTFPSRDDLLLFSPSGQGEFRALNARLPLNTDAYLS | |
| | LQELQGQDPTHLV | |
| CD8SS-TPOR/MPLR(478- | MALPVTALLLPLALLLHAARPSDPTRVETATETAWISLVTALHLVL | 273 |
| 582; S505N, W515K, K553R, | GLNAVLGLLLLRKQFPAHYRRLRHALWPSLPDLHRVLGQYLRDTA | |
| K573R).IL2Rb(393-433, 518- | ALSPPRATVSDTCEEVEPSLLEILPRSSERTPLPLLEDEGVAGAPTGS | |
| 551) | SPQPLQPLSGEDDAYCTFPSRDDLLLFSPSGQGEFRALNARLPLNT | |
| Underlined indicates CD8SS | DAYLSLQELQGQDPTHLV | |
| CD8SS- | MALPVTALLLPLALLLHAARPPGWFLDSPDRPWNPPTFSPALLVV | 274 |
| HAPD1.TPOR/MPLR(478- | TEGDNATFTCSFSNTSESFHVIWHRESPSGQTDTLAAFPEDRSQP | |
| 582; H499L, S505N, W515K, | GQDCRFRVTQLPNGRDFHMSVVRARRNDSGTYVCGVISLAPKIQI | |
| K553R, K573R).IL2Rb(339- | KESLRAELRVTERRAEVPTAHPSPSPRPAGQFQTLVSDPTRVETAT | |
| 379, 393-433, 518-551) | ETAWISLVTALLLVLGLNAVLGLLLLRKQFPAHYRRLRHALWPSLP | |
| Underlined indicates CD8SS | DLHRVLGQYLRDTAALSPPRATVSDTCEEVEPSLLEILPRSSERTPL | |
| Italicized marks high-affinity | PLLEQQDKVPEPASLSSNHSLTSCFTNQGYFFFHLPDALEIEACQD | |
| PD-1 ectodomain | EGVAGAPTGSSPQPLQPLSGEDDAYCTFPSRDDLLLFSPSGQGEF | |
| | RALNARLPLNTDAYLSLQELQGQDPTHLV | |
| CD8SS- | MALPVTALLLPLALLLHAARPPGWFLDSPDRPWNPPTFSPALLVV | 275 |
| HAPD1.TPOR/MPLR(478- | TEGDNATFTCSFSNTSESFHVIWHRESPSGQTDTLAAFPEDRSQP | |
| 582; S505N, W515K, K553R, | GQDCRFRVTQLPNGRDFHMSVVRARRNDSGTYVCGVISLAPKIQI | |
| K573R).IL2Rb(393-433, | KESLRAELRVTERRAEVPTAHPSPSPRPAGQFQTLVSDPTRVETAT | |
| 518-551) | ETAWISLVTALHLVLGLNAVLGLLLLRKQFPAHYRRLRHALWPSLP | |
| Underlined indicates | DLHRVLGQYLRDTAALSPPRATVSDTCEEVEPSLLEILPRSSERTPL | |
| CD8SSItalicized marks high- | PLLEDEGVAGAPTGSSPQPLQPLSGEDDAYCTFPSRDDLLLFSPSG | |
| affinity PD-1 ectodomain | QGEFRALNARLPLNTDAYLSLQELQGQDPTHLV | |

*The underlined LE and SR indicate exemplary peptide linkers.

### F. Expression of Chimeric Cytokine Receptors

Provided herein are polynucleotides encoding any one of the chimeric cytokine receptors provided herein. Likewise, provided herein are expression vectors comprising such polynucleotides. In some embodiments, the vector is a viral vector. In some embodiments, the vector is not a viral vector.

In some embodiments, the vector comprises a polynucleotide encoding a chimeric cytokine receptor, and a polynucleotide expressing a chimeric antigen receptor (CAR).

In some embodiments, expression of the chimeric cytokine receptor and the CAR are expressed as a single polypeptide chain, separated by a linker. FIGS. 2A, 3, 4A, 5A, 6A, 8A, and 11A show schematics of a vector that can be used to co-express the chimeric cytokine receptor and CAR of the disclosure. One or more STAT-recruiting domains may be joined in tandem to mimic signaling from one or more cytokines.

### II. CAR-Bearing Immune Cells

Provided herein are engineered immune cells comprising a polynucleotide encoding a chimeric antigen receptor and a chimeric cytokine receptor of the disclosure; and provided herein are engineered immune cells expressing a chimeric antigen receptor (CAR-I cell) and a chimeric cytokine receptor of the disclosure. Examples of immune cells include T-cells, e.g., alpha/beta T-cells and gamma/delta T-cells, B cells, natural killer (NK) cells, natural killer T (NKT) cells, invariant NKT cells, mast cells, myeloid-derived phagocytes, dendritic cells, killer dendritic cells, macrophages, and monocytes. Immune cells also refer to cells derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells.

Accordingly in some embodiments, provided herein are CAR-T cells comprising a chimeric cytokine receptor of the disclosure.

In some embodiments, a CAR can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain of a CAR specifically binds to a target of interest. The target of interest can be any molecule of interest, including, for example, without limitation any one or more of those presented in Table 8.

TABLE 8
List of targets of interest

BCMA
EGFRvIII
Flt-3
WT-1
CD20
CD23
CD30
CD38
CD70
CD33
CD133
MHC- WT1
TSPAN10
MHC-PRAME
Liv1
ADAM10
CHRNA2
LeY
NKG2D
CS1
CD44v6
ROR1
CD19
Claudin-18.2 (Claudin-18A2 or Claudin18 isoform 2)
DLL3 (Delta-like protein 3, Drosophila Delta homolog 3, Delta3)
Muc17 (Mucin17, Muc3, Muc3)
FAP alpha (Fibroblast Activation Protein alpha)
Ly6G6D (Lymphocyte antigen 6 complex locus protein G6d
c6orf23
G6D
MEGT1
NG25)
RNF43 (E3 ubiquitin-protein ligase RNF43, RING finger protein 43)
ErbB2 (HER2/neu)
carcinoembryonic antigen (CEA)
epithelial cell adhesion molecule (EpCAM)
epidermal growth factor receptor (EGFR)
CD40
disialoganglioside GD2
GD3
C-type lectin-like molecule-1 (CLL-1)
ductal-epithelial mucine
gp36
TAG-72
glycosphingolipids
glioma-associated antigen
β-human chorionic gonadotropin
alphafetoprotein (AFP)
lectin-reactive AFP
thyroglobulin
RAGE-1
MN-CA IX
human telomerase reverse transcriptase
RU1
RU2 (AS)
intestinal carboxyl esterase
mut hsp70-2
M-CSF
prostase
prostase specific antigen (PSA)
PAP
NY-ESO-1
LAGA-la
p53
prostein
PSMA
survivin and telomerase
prostate-carcinoma tumor antigen-1 (PCTA-1)
MAGE
ELF2M
neutrophil elastase
ephrin B2
CD22
insulin growth factor (IGFl)-l
IGF-II
IGFI receptor
mesothelin
a major histocompatibility complex (MHC) molecule presenting a
tumor-specific peptide epitope
5T4
O 1
Nkp30
tumor stromal antigens
the extra domain A (EDA) and extra domain B (EDB) of fibronectin
and the AI domain of tenascin-C (TnC AI) and fibroblast associated
protein (fap)
LRP6
melamona-associated Chondroitin Sulfate Proteoglycan (MCSP)
MARTI
MUC1
LMP2
Idiotype
NY-ESO-1
Ras mutant
gp100
proteinase 3
bcr-abl
tyrosinase
hTERT
EphA2
ML-TAP
ERG
NA17
PAX3
ALK
Androgen receptor
a lineage-specific or tissue specific antigen such as CD3
CD4
CD8
CD24
CD25
CD34
CD79
CD116
CD117
CD135
CD123
CD138
CTLA-4
B7-1 (CD80)
B7-2 (CD86)
endoglin
a major histocompatibility complex (MHC) molecule
MUC16
PSCA
Trop2
CD171 (L1CAM)
CA9
STEAP1
VEGFR2

In some embodiments, the extracellular ligand-binding domain of a CAR comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988) (e.g. glycine-serine containing linkers). In general, linkers can be short, flexible polypeptides and are generally comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the invention is responsible for intracellular signaling following the binding of extracellular ligand-binding domain to the target resulting in the activation of the immune cell and immune response (Signals 1 and/or 2). The intracellular signaling domain has the ability to activate at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain. In some embodiments the intracellular signaling domain of the CAR of the invention comprises a domain of a co-stimulatory molecule.

In some embodiments, the intracellular signaling domain of a CAR of the invention comprises a part of co-stimulatory molecule selected from the group consisting of fragment of 41BB (GenBank: AAA53133.) and CD28 (NP_006130.1).

CARs are expressed on the surface membrane of the cell. Thus, the CAR comprises a transmembrane domain. Suitable transmembrane domains for a CAR disclosed herein have the ability to (a) be expressed at the surface of a cell, preferably an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8a chain (e.g., NP_001139345.1). The transmembrane domain can further comprise a stalk domain between the extracellular ligand-binding domain and said transmembrane domain. A stalk domain may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, said transmembrane and hinge domains comprise a part of human CD8α chain. In some embodiments, CARs disclosed herein can comprise an extracellular ligand-binding domain that specifically binds BCMA, CD8α human hinge and transmembrane domains, the CD3ζ signaling domain, and 4-1BB signaling domain.

In some embodiments, a CAR can be introduced into an immune cell as a transgene via a plasmid vector. In some embodiments, the plasmid vector can also contain, for example, a selection marker which provides for identification and/or selection of cells which received the vector.

Table 7 provides exemplary sequences of CAR components that can be used in the CARs disclosed herein.

TABLE 7
Exemplary Sequences

| | | SEQ |
|---|---|---|
| Domain | Amino acid sequence | ID NO: |
| V5 epitope tag | KPIPNPLLGLDST | 152 |
| 2173 scFv | EIQLVQSGAEVKKPGESLRISCKGSGFNIEDYYIR | 153 |
| | WVRQMPGKGLEWMGRIDPENDETKYGPIFQGH | |
| | VTISADTSINTVYLQWSSLKASDTAMYYCAFRG | |
| | GVYWGQGTTVTVSSGGGGSGGGGSGGGGSGGG | |
| | GSDVVMTQSPDSLAVSLGERATINCKSSQSLLDS | |
| | DGKTYLNWLQQKPGQPPKRLISLVSKLDSGVPD | |
| | RFSGSGSGTDFTLTISSLQAEDVAVYYCWQGTHF | |
| | PGTFGGGTKVEIK | |
| CD8 hinge and | TTTPAPRPPTPAPTIASQPLSLRPEACRPAAGGAV | 154 |
| transmembrane | HTRGLDFACDIYIWAPLAGTCGVLLLSLVITLYC | |
| 4-1BB intracellular | KRGRKKLLYIFKQPFMRPVQTTQEEDGCSCRFPE | 155 |
| signaling | EEEGGCEL | |
| CD3z intracellular | RVKFSRSADAPAYQQGQNQLYNELNLGRREEYD | 156 |
| signaling | VLDKRRGRDPEMGGKPRRKNPQEGLYNELQKD | |
| | KMAEAYSEIGMKGERRRGKGHDGLYQGLSTAT | |
| | KDTYDALHMQALPPR | |
| BFP (blue fluorescent | MSELIKENMHMKLYMEGTVDNHHFKCTSEGEG | 157 |
| protein) | KPYEGTQTMRIKVVEGGPLPFAFDILATSFLYGS | |
| | KTFINHTQGIPDFFKQSFPEGFTWERVTTYEDGG | |
| | VLTATQDTSLQDGCLIYNVKIRGVNFTSNGPVM | |
| | QKKTLGWEAFTETLYPADGGLEGRNDMALKLV | |
| | GGSHLIANIKTTYRSKKPAKNLKMPGVYYVDYR | |
| | LERIKEANNETYVEQHEVAVARYCDLPSKLGHK | |
| | LN | |
| P2A | GSGATNFSLLKQAGDVEENPGP | 158 |

In some embodiments, the CAR-immune cell (e.g., CAR-T cell) of the disclosure comprises a polynucleotide encoding a suicide polypeptide, such as for example RQR8. See, e.g., WO2013153391A, which is hereby incorporated by reference in its entirety. In some embodiments, a suicide polypeptide is expressed on the surface of the cell. In some embodiments, a suicide polypeptide is included in the CAR construct. In some embodiments, a suicide polypeptide is not part of the CAR construct.

In some embodiments, the extracellular domain of any one of CARs disclosed herein may comprise one or more epitopes specific for (specifically recognized by) a monoclonal antibody. These epitopes are also referred to herein as mAb-specific epitopes. Exemplary mAb-specific epitopes are disclosed in International Patent Publication No. WO 2016/120216, which is incorporated herein in its entirety. In these embodiments, the extracellular domains of the CARs comprise antigen binding domains that specifically bind to a target of interest and one or more epitopes that bind to one or more monoclonal antibodies (mAbs). CARs comprising the mAb-specific epitopes can be single-chain or multi-chain.

The inclusion of epitopes specific for monoclonal antibodies in the extracellular domain of the CARs described herein allows sorting and depletion of engineered immune cells expressing the CARs. In some embodiments, allowing for depletion provides a safety switch in case of deleterious effects, e.g., upon administration to a subject.

Methods of preparing immune cells for use in immunotherapy are also provided herein. In some embodiments, the methods comprise introducing a chimeric cytokine receptor and a CAR into immune cells, and expanding the cells. In some embodiments, the invention relates to a method of engineering an immune cell comprising: providing a cell and expressing a chimeric cytokine receptor, and expressing at the surface of the cell at least one CAR. In some embodiments, the method comprises: transfecting the cell with at least one polynucleotide encoding a chimeric cytokine receptor, and at least one polynucleotide encoding a CAR, and expressing the polynucleotides in the cell. In some embodiments, the method comprises: transfecting the cell with at least one polynucleotide encoding a chimeric cytokine receptor, at least one polynucleotide encoding a CAR, and expressing the polynucleotides in the cell. In some embodiments, the chimeric cytokine receptor and the CAR reside on one polynucleotide.

In some embodiments, the one or more polynucleotides encoding the chimeric cytokine receptor and CAR are present in one or more expression vectors for stable expression in the cells. In some embodiments, the polynucleotides are present in viral vectors for stable expression in the cells. In some embodiments, the one or more polynucleotides are inserted into the cellular genome by random integration, and in other embodiments, inserted into specific locations of the cellular genome by site-specific integration. In some embodiments, the viral vectors may be for example, lentiviral vectors or adenoviral vectors. In some embodiments, the one or more polynucleotides are present in non-viral vectors.

In some embodiments, polynucleotides encoding polypeptides according to the present disclosure can be mRNA which is introduced directly into the cells, for example by electroporation. In some embodiments, CytoPulse electroporation technology, such as PulseAgile, can be used to transiently permeabilize living cells for delivery of material into the cells (e.g. U.S. Pat. No. 6,078,490; PCT/US2011/000827; and PCT/US2004/005237). Parameters can be modified in order to determine conditions for high transfection efficiency with minimal mortality.

Also provided herein are methods of transfecting an immune cell, e.g. a T-cell. In some embodiments, the method comprises: contacting a T-cell with RNA and applying to the T-cell an agile pulse sequence. In some embodiments, a method of transfecting an immune cell (e.g. T-cell) comprising contacting the immune cell with RNA and applying to the cell an agile pulse sequence.

In some embodiments, the method can further comprise a step of genetically modifying a cell by inactivating at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, CD52, GR, deoxycytidine kinase (DCK), TGF-B, and CTLA-4. In some embodiments the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or CRISPR-based endonuclease (e.g. Cas-9 or Cas12a).

In another aspect, a step of genetically modifying cells can comprise: modifying immune cells (e.g. T-cells) by inactivating at least one gene expressing a target for an immunosuppressive agent, and; expanding the cells, optionally in presence of the immunosuppressive agent.

In some embodiments, the engineered immune cells (e.g. T-cells) provided herein exhibit improved cytotoxicity, increased expansion, and/or increased levels of memory phenotype markers upon contact with a TGF-β ligand or anti-TGF-β-receptor antibody that binds to the binding domain of the chimeric cytokine receptor relative to engineered immune cells that do not express the chimeric cytokine receptor.

In some embodiments, the engineered immune cells (e.g. T-cells) provided herein exhibit (i) increased in vivo persistence, (ii) increased STAT activation, (iii) increased cytotoxicity, (iv) increased levels of memory phenotype markers, (v) increased expansion (proliferation), or combinations of these functional features, upon contact with a TGF-β ligand or anti-TGF-β-receptor antibody that binds to the binding domain of the chimeric cytokine receptor relative to engineered immune cells that do not express the chimeric cytokine receptor. In some embodiments, the improvement in the one or more functional features described herein is dose-dependent, i.e., the functional activity of the immune cell comprising the chimeric cytokine receptors increases upon contact with increasing doses of the PD-L1/PD-L2/TGF-B or an antibody to the respective receptor. In some embodiments, STATs activated by the engineered immune cell comprising one or more chimeric cytokine receptors disclosed are STAT1, STAT2, STAT3, STAT4, STAT5, STAT6, or combinations thereof. In one embodiment, memory phenotype markers that are increased or maintained by the immune cell comprising the chimeric cytokine receptor of the disclosure include stem cell memory (Tscm) markers and central memory (Tcm) markers.

In some embodiments, the improvement in one or more functional features exhibited by an engineered immune cell comprising a chimeric cytokine receptor provided herein is at least about 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 25 fold, 30 fold, 40 fold, 50 fold, 60 fold, 70 fold, 80 fold, 90 fold, 100 fold, 125 fold, 150 fold, 200 fold, 250 fold, 300 fold, 350 fold, 400 fold, 450 fold, or even about 10-500 fold, including values and ranges therebetween, compared to an immune cell that does not express the chimeric cytokine receptor.

In some embodiments, the improvement in one or more functional features exhibited by an engineered immune cell comprising a chimeric cytokine receptor provided herein is at least about 10%, 20%, 25%, 30%, 35%, 40%, 45%, 50%, 55% 60%, 70%, 75%, 80%, 90%, 100%, 125%, 150%, 200%, 250%, 300%, 350%, 400%, or even about 80%-500%, including values and ranges therebetween, compared to an engineered immune cell that does not express the chimeric cytokine receptor.

### III. Therapeutic Methods

Provided herein are pharmaceutical compositions comprising cells bearing the chimeric cytokine receptors and CARs of the disclosure.

Engineered chimeric cytokine receptor-bearing and CAR-bearing immune cells (e.g. T-cells) obtained by the methods described above, or cell lines derived from such engineered immune cells, can be used as a medicament. In some embodiments, such a medicament can be used for treating a disorder such as for example a viral disease, a bacterial disease, a cancer, an inflammatory disease, an immune disease, or an aging-associated disease. In some embodiments, the cancer is a solid cancer. In some embodiments the cancer is a liquid cancer. The cancer can be selected from the group consisting of gastric cancer, sarcoma, lymphoma, leukemia, head and neck cancer, thymic cancer, epithelial cancer, salivary cancer, liver cancer, stomach cancer, thyroid cancer, lung cancer, small cell lung cancer, ovarian cancer, breast cancer, prostate cancer, esophageal cancer, pancreatic cancer, glioma, glioblastoma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, engineered immune cells, or cell line derived from the engineered immune cells, can be used in the manufacture of a medicament for treatment of a disorder in a subject in need thereof. In some embodiments, the disorder can be, for example, a cancer, an autoimmune disorder, or an infection.

Also provided herein are methods for treating subjects in need of such treatment.

As used herein, the term “subject” refers to any vertebrate including, without limitation, humans and other primates (e.g., chimpanzees, cynomologous monkeys, and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In exemplary embodiments, the subject is a human.

In some embodiments the method comprises providing immune cells of the disclosure, bearing the chimeric cytokine receptors and CARs described herein to a subject in need thereof.

In some embodiments, chimeric cytokine receptor and CAR-bearing T-cells of the invention can undergo robust in vivo T-cell expansion and can persist for an extended amount of time.

Methods of treatment of the invention can be ameliorating, curative or prophylactic. The method of the invention may be either part of an autologous immunotherapy or part of an allogenic immunotherapy treatment.

In another aspect, the invention provides a method of inhibiting tumor growth or progression in a subject who has a tumor, comprising administering to the subject an effective amount of chimeric cytokine receptor-expressing and CAR-expressing immune cells as described herein. In another aspect, the invention provides a method of inhibiting or preventing metastasis of cancer cells in a subject, comprising administering to the subject in need thereof an effective amount of engineered immune cells as described herein. In another aspect, the invention provides a method of inducing tumor regression in a subject who has a tumor, comprising administering to the subject an effective amount of engineered immune cells as described herein. In some embodiments, the subject is further administered with an anti-TGF-βR antibody, in particular, an anti-TGF-βR2 antibody.

In some embodiments, the engineered T-cells herein can be administered parenterally in a subject. In some embodiments, the engineered T-cells disclosed herein can be administered intravenously in a subject.

Also provided is the use of any of the engineered T-cells provided herein in the manufacture of a medicament for the treatment of cancer or for inhibiting tumor growth or progression in a subject in need thereof.

In some embodiments, treatment can be administrated into subjects undergoing an immunosuppressive treatment. Indeed, the invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the subject. The administration of the cells or population of cells according to the invention may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous or intralymphatic injection, or intraperitoneally. Cells bearing the chimeric cytokine receptors and/or CARs of the disclosure or the pharmaceutical compositions thereof may be administered via one or more of the following routes of administration: intravenous, intraocular, intravitreal, intramuscular, subcutaneous, topical, oral, transdermal, intraperitoneal, intraorbital, by implantation, by inhalation, intrathecal, intraventricular, via the ear, or intranasal.

In some embodiments the administration of the cells or population of cells (bearing the chimeric cytokine receptors and CARs of the disclosure) can comprise administration of, for example, about 10⁴ to about 10⁹ cells per kg body weight including all integer values of cell numbers within those ranges. In some embodiments the administration of the cells or population of cells can comprise administration of about 10⁴ to 10⁵ cells per kg body weight, 10⁵ to 10⁶ cells per kg body weight, 10⁶ to 10⁷ cells per kg body weight, 10⁷ to 10⁸ cells per kg body weight, or 10⁸ to 10⁹ cells per kg body weight. The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or condition is within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

The methods can further comprise administering one or more agents to a subject prior to administering the engineered immune cells bearing a CAR and a chimeric cytokine receptor provided herein. In certain embodiments, the agent is a lymphodepleting (preconditioning) regimen. For example, methods of lymphodepleting a subject in need of such therapy comprise administering to the subject specified beneficial doses of cyclophosphamide (between 200 mg/m²/day and 2000 mg/m²/day, about 100 mg/m²/day and about 2000 mg/m²/day; e.g., about 100 mg/m²/day, about 200 mg/m²/day, about 300 mg/m²/day, about 400 mg/m²/day, about 500 mg/m²/day, about 600 mg/m²/day, about 700 mg/m²/day, about 800 mg/m²/day, about 900 mg/m²/day, about 1000 mg/m²/day, about 1500 mg/m²/day or about 2000 mg/m²/day) and specified doses of fludarabine (between 20 mg/m²/day and 900 mg/m²/day, between about 10 mg/m²/day and about 900 mg/m²/day; e.g., about 10 mg/m²/day, about 20 mg/m²/day, about 30 mg/m²/day, about 40 mg/m²/day, about 40 mg/m²/day, about 50 mg/m²/day, about 60 mg/m²/day, about 70 mg/m²/day, about 80 mg/m²/day, about 90 mg/m²/day, about 100 mg/m²/day, about 500 mg/m²/day or about 900 mg/m²/day). An exemplary dosing regimen involves treating a subject comprising administering daily to the patient about 300 mg/m²/day of cyclophosphamide in combination or before or after administering about 30 mg/m²/day of fludarabine for three days prior to administration of a therapeutically effective amount of engineered immune cells to the patient.

In some embodiments, notably in the case when the engineered cells provided herein have been gene edited to eliminate or minimize surface expression of CD52, lymphodepletion further comprises administration of an anti-CD52 antibody, such as alemtuzumab. In some embodiments, the CD52 antibody is administered at a dose of about 1-20 mg/day IV, e.g., about 13 mg/day IV for 1, 2, 3 or more days. The antibody can be administered in combination with, before, or after administration of other elements of a lymphodepletion regime (e.g., cyclophosphamide and/or fludarabine).

In certain embodiments, compositions comprising CAR-expressing immune effector cells disclosed herein may be administered in conjunction with any number of chemotherapeutic agents.

### IV. Kits and Articles of Manufacture

The present disclosure provides kits comprising any one or more of the chimeric cytokine receptors and chimeric cytokine receptor-bearing cells described herein, and pharmaceutical compositions thereof. The present disclosure also provides articles of manufacture comprising any one or more of the chimeric cytokine receptors and chimeric cytokine receptors-bearing CAR-I-cells described herein, pharmaceutical compositions thereof, and kits described herein.

The following examples are included for illustrative purposes and are not intended to limit the scope of the disclosure.

All patent and non-patent documents referenced throughout this disclosure are incorporated by reference herein in their entirety for all purposes.

### EXAMPLES

### Example 1: Construction and Testing of Chimeric Cytokine Receptor-CAR Constructs Having a TGFβR1 or TGFβR2 Dominant Negative Truncation

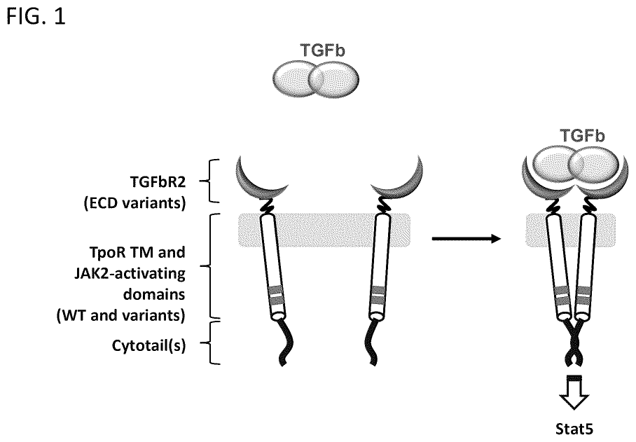

FIG. 1 shows a schematic of the inducible chimeric cytokine receptor of the disclosure. To couple simultaneous TGF-β engagement with cytokine signaling, a chimeric cytokine receptor was constructed, composed of the following modules: (i) a binding domain comprising an extracellular portion of a TGF-β receptor, or a TGF-β antigen binding domain; (ii) a transmembrane domain with an intracellular portion having a JAK2-activating domain and (iii) STAT-recruiting domains comprising STAT-recruiting (STAT-activating) domains from cytokine receptor tails (cytotails). As shown as an example in FIG. 1, the binding domain comprises the extracellular domain of TGFβR2.

A HEK293T cell reporter assay was used to test the inducibility and magnitude of cytokine signaling using chimeric cytokine receptors for either neutralizing the TGF-β signaling or activating the STAT response, which can be used as a surrogate measurement for the cytokine ICD activation and cytokine signaling. Briefly, 20,000 HEK293T-cells were plated into each well of a poly-L-lysine-coated 96-well flat-bottom plate and cultured overnight at 37° C. with 5% CO₂. A chimeric cytokine receptor-CAR construct (2.5 ng), a TGF-β or STAT-response element that drives Firefly Luciferase (100 ng; Promega), and Renilla Luciferase control reporter vector (1 ng; Promega) were mixed to a final volume of 5 μl in Opti-MEM (Gibco) (“DNA mix”).

Cells were transfected with a BFP-EGFRvIII CAR construct where a BFP gene is in place of the chimeric cytokine receptor as a negative control. A dominant negative truncation of TGFβR2 (“TGFβR2 DN”) and a dominant negative truncation of TGFβ1 (“TGFβR1 DN”) were also constructed as additional controls to examine dominant negative effects in the absence of an intracellular cytokine signal. After incubating the DNA mixes with premixed 0.3 μl Lipofectamine 2000 (Invitrogen) and 5 μl Opti-MEM at room temperature for 20 minutes, the mixture having a total volume of 10 μl was added to each well containing HEK293T cells. One day after transfection, a commercially available TGF-β1 ligand (BioLegend, hereinafter in Examples 1-4 referred to as “TGF-β”) was added to the culture for stimulation, to various final concentrations. After 20-24 hours of stimulation, TGF-β or STAT5 reporter activity was evaluated using the Dual-Glo Luciferase Assay System (Promega). Fold induction of TGF-β or STAT5 reporter activity was normalized to that of HEK293T cells that were transfected with only a reporter vector, and left untreated.

FIG. 2A shows a schematic of the lentiviral vector used to co-express the dominant negative truncations of the TGFβR1 or TGFβR2 chimeric cytokine receptor with the 2^{nd} generation EGFRvIII CAR.

FIG. 2B shows the inhibition of the TGF-β signaling by expression of either the TGFβR1 DN or TGFβR2 DN (shown by FIG. 2A, and comprising the amino acid sequences of SEQ ID NOs 2 and 3, respectively). The data shown indicates that the TGFβR2 DN chimeric cytokine receptor inhibits TGF-β signaling induced by the TGF-β ligand (up to 100 ng/ml), with higher efficacy than the TGFβR1 DN. This is likely due to the higher affinity of TGFβR2 binding to the TGF-β ligand in comparison to the binding affinity of TGFβR1 binding to the TGF-β ligand (described in Groppe et al., 2008, Mol. Cell, 29(2):157-68). Consequentially, the designs of the following examples focus on the chimeric cytokine receptors having the binding domain of TGFβR2.

### Example 2: Designs and Testing of Inducible Chimeric Cytokine Receptors Using TGFbR2

A chimeric cytokine receptor was constructed, as briefly described when referring to FIG. 1, having a binding domain derived from TGFβR2 (“TGFβR2 chimeric cytokine receptor”). To investigate the utility of the TGFβR2 chimeric cytokine receptor in the context of CAR-T cells, variants of TGFβR2 extracellular domains (ECD) and variants of TPOR transmembrane (TM) domains were constructed. Fusions of each TGFβR2 ECD variant, each TPOR TM domain variant, and the intracellular domains (ICD) of desired cytokine receptors were cloned into a lentiviral vector encoding a 2^{nd} generation EGFRvIII-specific CAR (2173scFv; described in Sci Transl Med. 2015 Feb. 18; 7(275): 275ra22), and the activity of these receptor variants was tested. To permit stoichiometric co-expression of the chimeric cytokine receptor and the CAR, both genes were linked via a P2A peptide (“chimeric cytokine receptor-CAR construct”). To facilitate the detection of transduced cells, a v5 epitope tag (SEQ ID NO: 152) was inserted between the scFv and CD8 hinge domain.

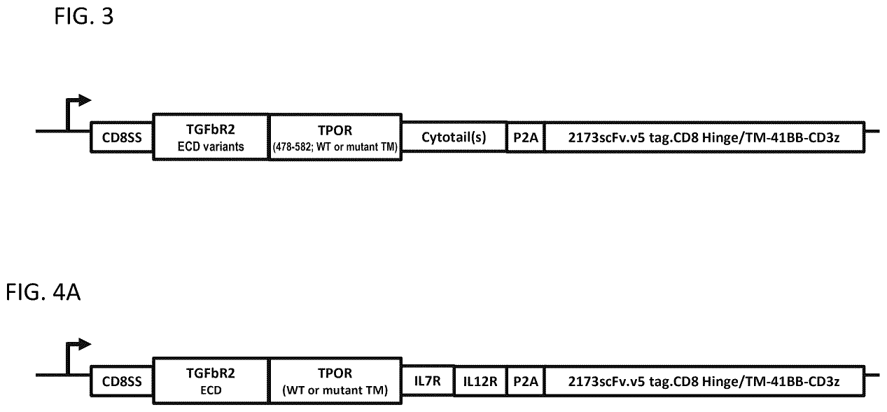

FIG. 3 shows a general schematic of the lentiviral vector used to co-express the TGFβR2 chimeric cytokine receptor with the 2^{nd} generation EGFRvIII CAR. One or more cytotails or recruiting domains may be joined in tandem to mimic signaling from one or more cytokines.

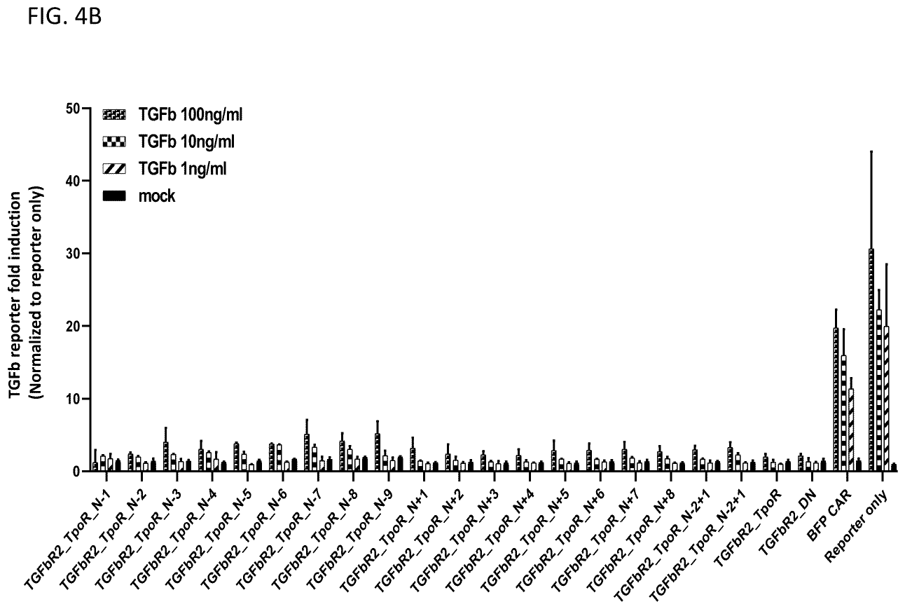

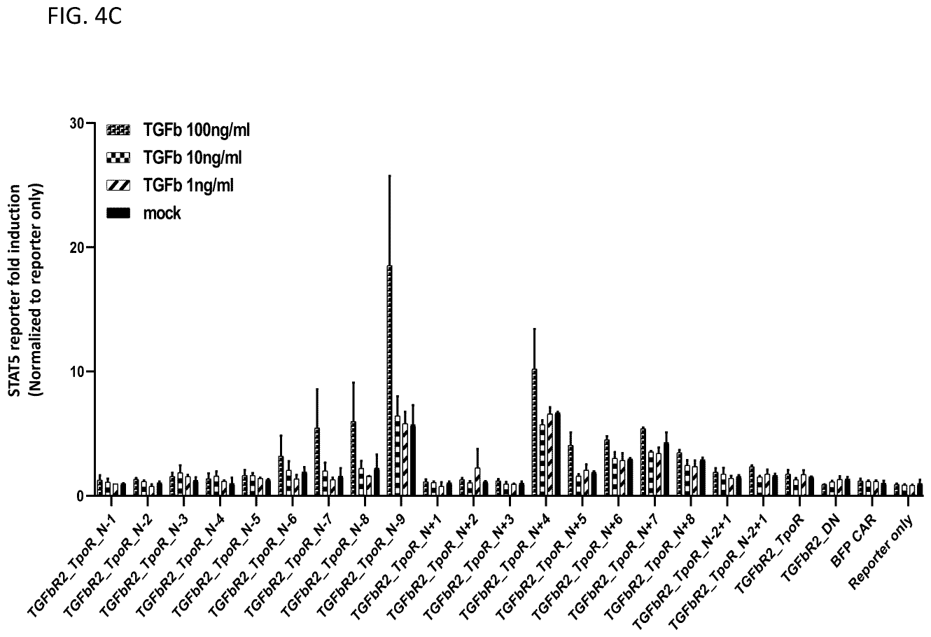

FIGS. 4A-4C show the inhibition of TGF-β signaling by the overexpression of chimeric cytokine receptors constructed using TGFβR2. The lentiviral vectors used were constructed similarly as described in Example 1. FIG. 4A shows a schematic of the prototypic lentiviral vector used, bearing the IL7R(316-459) and the IL12Rb2(775-825) cytotail or recruiting domains to mimic IL7 and IL12 signaling in CAR-T-cells. A variety of truncations in the transmembrane domain of the TpoR cassette were designed (as shown in Table 3). The capacity of these truncations to regulate cytokine signaling was determined. FIG. 4B shows TGF-β signaling activity determined by a luciferase reporter assay. All the tested chimeric cytokine receptors constructed using TGFβR2 were shown to be able to inhibit TGF-β signaling as they compete for binding via the extracellular domain of TGFβR2 in these engineered chimeric receptors. FIG. 4C shows the activation of the chimeric cytokine receptors of FIG. 4A in the presence of TGF-β. The activation of the chimeric cytokine receptor is measured by STAT reporter activity. Several variants were identified to have the ability to induce cytokine signaling by a TGF-β ligand. The amino acid sequences of the transmembrane domains listed in the X-axes of FIGS. 4B-4C are SEQ ID NO: 29 to SEQ ID NO: 40 and SEQ ID NO: 50 to SEQ ID NO: 57, presented in Table 3.

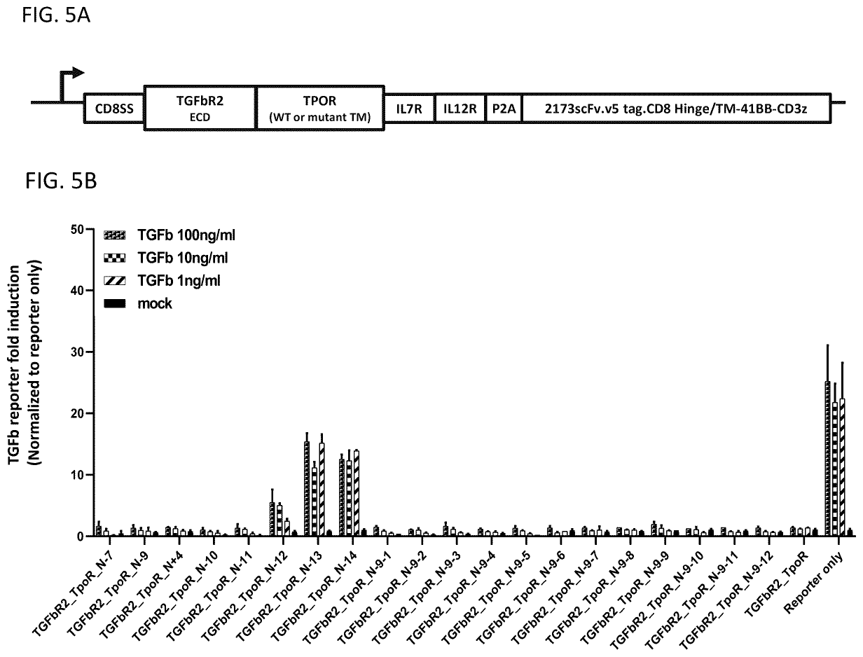

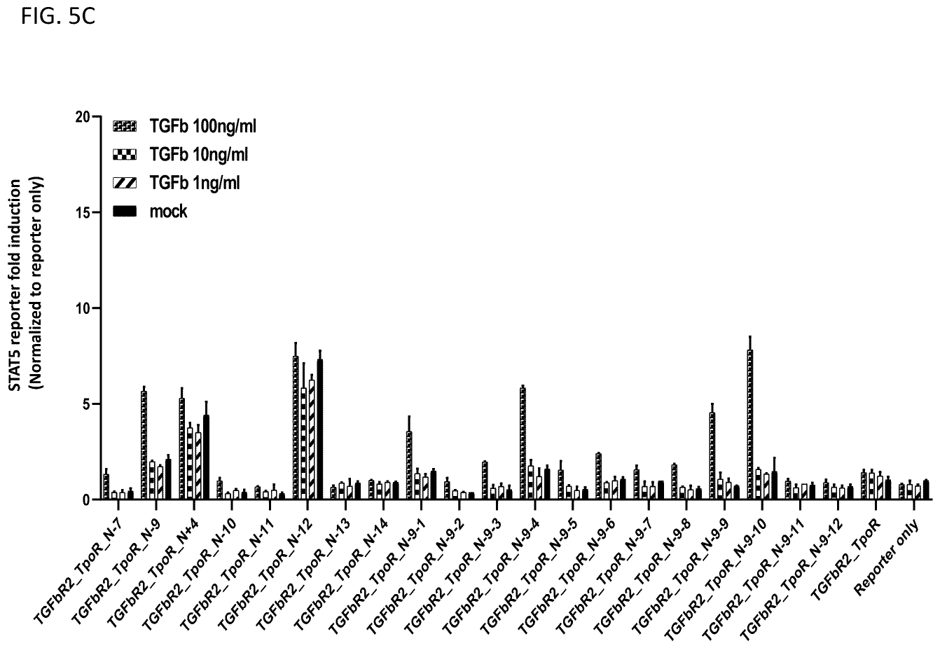

FIGS. 5A-5C show the inhibition of TGF-β signaling by the overexpression of additional chimeric cytokine receptors constructed using TGFβR2. The lentiviral vectors used were constructed similarly as described when referring to FIGS. 4A-4C, and Example 1. FIG. 5A shows a schematic of the prototypic lentiviral vector used, having truncations in the binding domain.

Again, additional truncations (N−10, N-11, N-12, etc.) in the TM domain of TpoR cassette were designed (as shown in Table 3), and their capacity to regulate cytokine signaling was determined. FIG. 5B shows TGF-β signaling activity determined by a luciferase reporter assay. Most of the chimeric cytokine receptors tested were shown to be able to inhibit TGF-β signaling (other than the N−12, N-13, N-14 TM truncations, which show less extent of inhibition). FIG. 5C shows the activation of the chimeric cytokine receptors of FIG. 5A in the presence of TGF-β. The activation of the chimeric cytokine receptors is measured by the STAT reporter activity. Several variants were identified to have the ability to induce cytokine signaling by a TGF-β ligand. The amino acid sequences of the transmembrane domains listed in the X-axes of FIGS. 5B-5C are SEQ ID NOs: 29, 38, 53, SEQ ID NO: 40 to SEQ ID NO: 44, and SEQ ID NO: 61 to SEQ ID NO: 72, presented in Table 3.

### Example 3: Modifications of the Chimeric Cytokine Receptor Binding Domain and Testing of the Constructed Chimeric Cytokine Receptors

In the absence of TGFβR2, TGFβR1 interacts with the TGF-β ligand with very low affinity. Once the ECD of TGFβR2 binds to the TGF-β ligand, the binary complex has an extended interface to efficiently recruit TGFβR1 to form the ternary complex. The engineered TGFβR2 chimeric cytokine receptor can also engage endogenous TGFβR1, which may sterically intervene the intended signaling though the cytokine receptor ICDs. To abrogate interaction between the TGFβR2 chimeric cytokine receptors and TGFβR1, several variants for the TGFβR1 cassette were designed, and modifications that can enhance cytokine signaling while inhibiting the TGF-β signaling were identified.

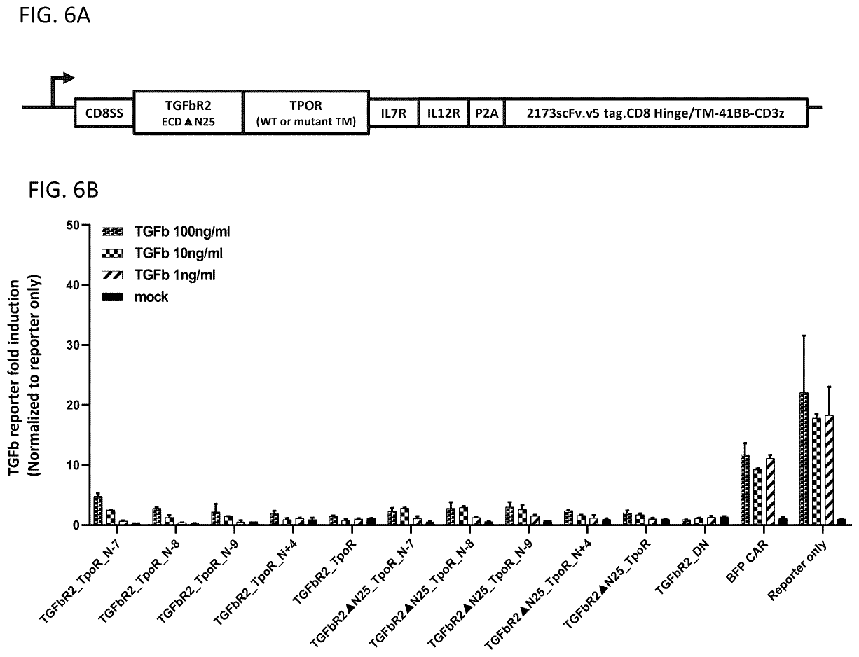

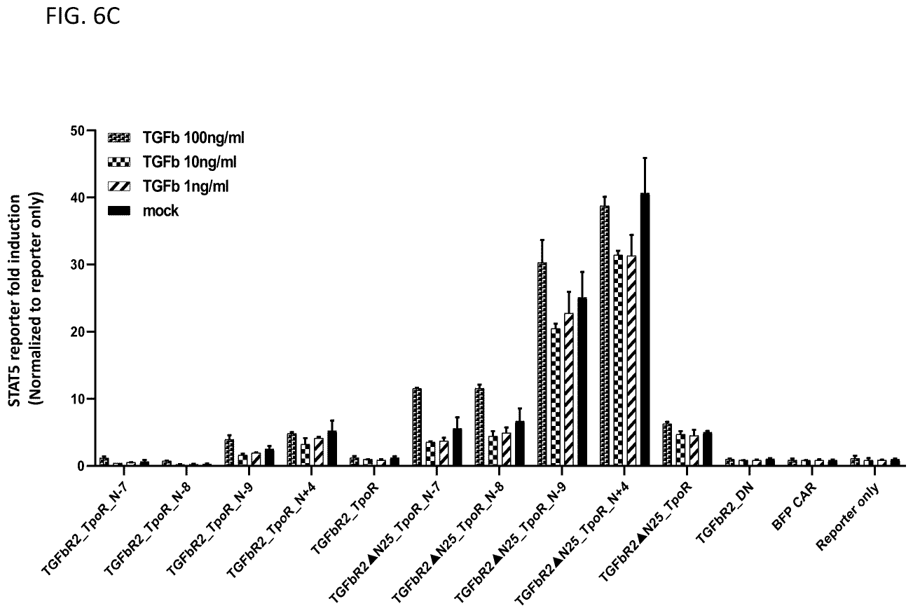

FIGS. 6A-6C show the inhibition of TGF-β signaling by the expression of chimeric cytokine receptors constructed with TGFβR2 having modifications. FIG. 6A shows a schematic of the modification introduced into the TGFβR2 cassette. The lentiviral vectors used were constructed similarly as described in Example 1. To abolish the engagement of TGFβR1, a truncation (ΔN25) was introduced into the TGFβR2 binding domain based on the previous constructs described when referring to FIGS. 6A-6C (“TGFβR2ΔN25”), and the inhibition of TGF-β signaling was tested by a TGF-β reporter assay. FIG. 6B shows that the TGFβR2ΔN25 chimeric cytokine receptors still retained the ability to inhibit TGF-β signaling. FIG. 6C shows the activation of the chimeric cytokine receptors of FIG. 6A, evaluated by the STAT reporter activity. The amino acid sequences of the transmembrane domains listed in the X-axes of FIGS. 6B-6C are SEQ ID NOs: 29, 38, 39, 40, and 53, with a binding domain sequence of SEQ ID NO: 4 or SEQ ID NO: 3, presented in Tables 1 and 3.

It was determined that the truncation in the TGFβR2 binding domain enhances the cytokine signaling by 5~10 fold, even in the absence of a TGF-β ligand. Interestingly, the ΔN25 truncation was able to enhance the signaling synergistically with the TpoR TM truncations (e.g. N−7, N−8, N−9 and N+4). This combinatorial use of the TGFβR2 binding domain and TpoR TM truncation mutants represents a novel approach for simultaneously inhibiting immunosuppressive TGF-β signaling while transmitting immune-potentiating cytokine signaling.

### Example 4: Design and Testing of the Constitutively Active Chimeric Cytokine Receptor

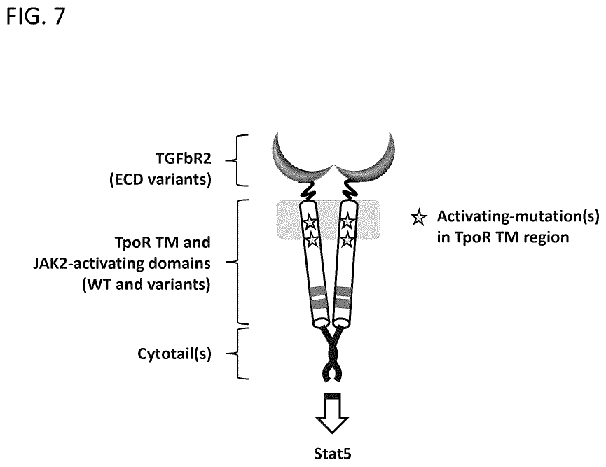

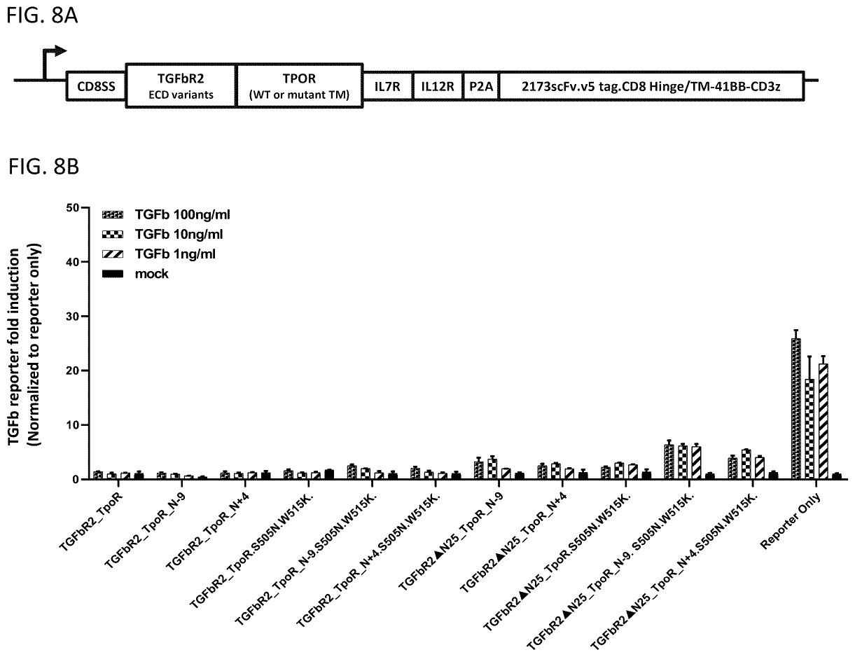

FIG. 7 shows a schematic of the constitutively active chimeric cytokine receptor. To enable constitutively active signaling of the cytokine intracellular domains, double mutations that inherently dimerize the TPOR transmembrane domain and activate the JAK-STAT pathway were introduced in the TPOR transmembrane domain. As shown as an exemplary construct in FIG. 7, the binding domain comprises the extracellular portion of TGFβR2; exemplary transmembrane domains may comprise the SEQ ID NOs 29, 40, 53 or 60.

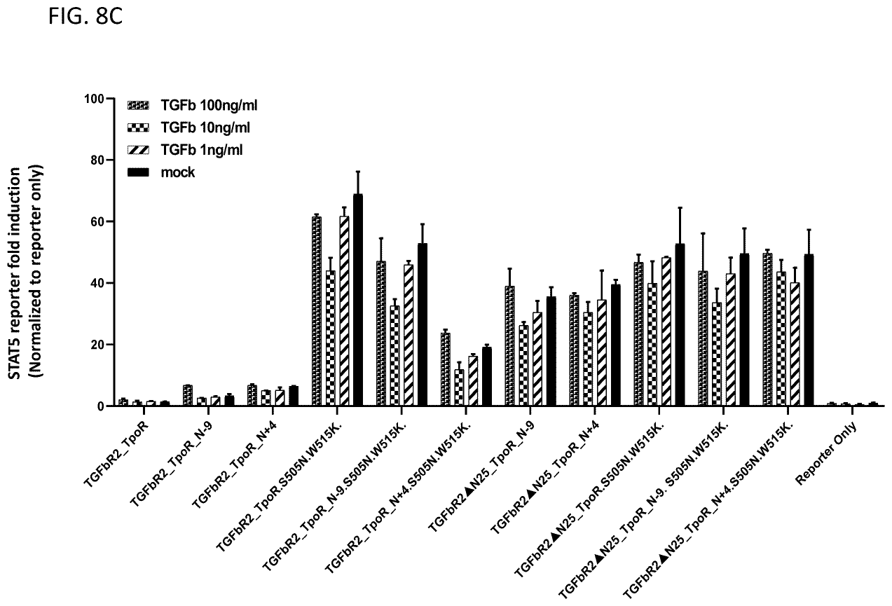

FIGS. 8A-8C show the design and tested function of the constitutively active chimeric cytokine receptors. The lentiviral vectors used were constructed similarly as described in Example 1. A double mutant (S505N, W515K) was introduced into the TPOR cassette to enforce the dimerization and activation of the receptors, in combination with variants that had shown the most promising functionality (e.g. TGFβR2ΔN25, N−9 and N+4 truncations in the TPOR cassette). FIG. 8A shows a schematic of the modification introduced into the TGFβR2 cassette. FIG. 8B shows the inhibition of TGF-β signaling by the expression of the chimeric cytokine receptors of FIG. 8A. FIG. 8C shows the activation of cytokine signaling via the chimeric cytokine receptors of FIG. 8A, measured by the STAT5 reporter activity in 293 cells. Compared to the parental designs (TGFβR2_TpoR, TGFβR2_TpoR_N−9, and TGFβR2_TpoR_N+4), receptors with the S505N and W515K mutations display substantial cytokine signaling (e.g. TGFβR2_TpoR.S505.W515K, TGFβR2_TpoR_N−9.S505N.W515K). The sequences of the transmembrane domains listed in the X-axes of FIGS. 8B-8C comprise SEQ ID NOs: 40, 53, 59, and 60 with a binding domain sequence of SEQ ID NO: 4 or SEQ ID NO: 3, presented in Tables 1 and 3.

### Example 5 Testing of Additional Chimeric Cytokine Receptors

FIG. 10A shows additional design of inducible TGF-β-driven chimeric cytokine receptors. Constructs with further truncations in the transmembrane domain to decrease the flexibility between the ECD and intracellular signaling domain were made. FIG. 10B shows the inhibition of endogenous TGF-β signaling as determined by luciferase assay by the overexpression of TGF-β-driven chimeric cytokine receptors shown in FIG. 10A in 293 cells in the presence of different concentrations of TGF-β. Some of the constructs also carry the ΔN25 deletion in the ECD domain (“TGF-βR2ΔN25”). All TGF-β-driven chimeric cytokine receptors in FIG. 10B inhibited the endogenous TGF-β signaling, although clones with the ΔN25 deletion showed slightly decreased inhibition of the TGF-β signaling. The data in FIG. 10C show the activation of chimeric cytokine receptors in the presence of TGF-β at various concentrations. Chimeric receptors with the deletion in the transmembrane domain and ΔN25 deletion induced STAT5 signaling in the presence of TGF-β.

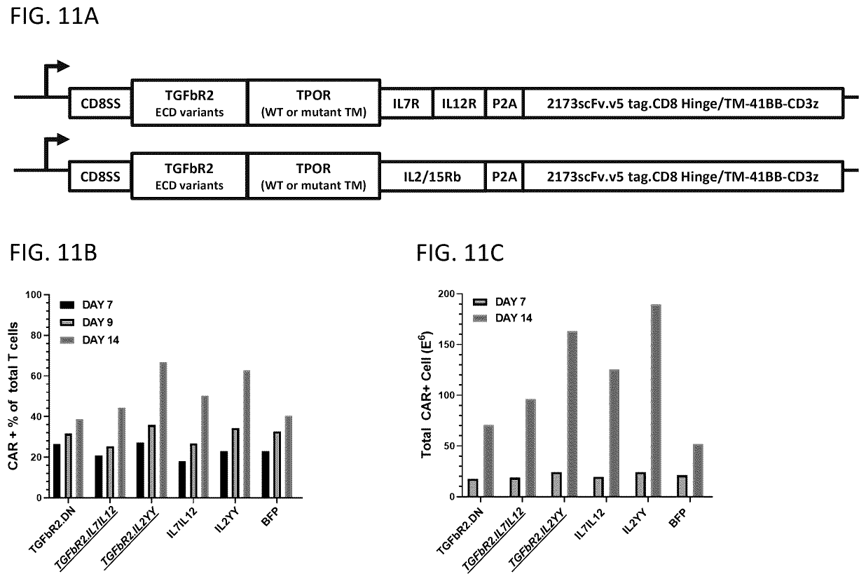

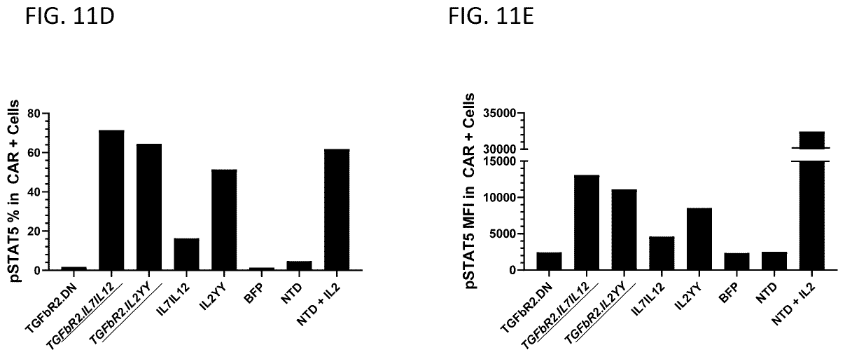

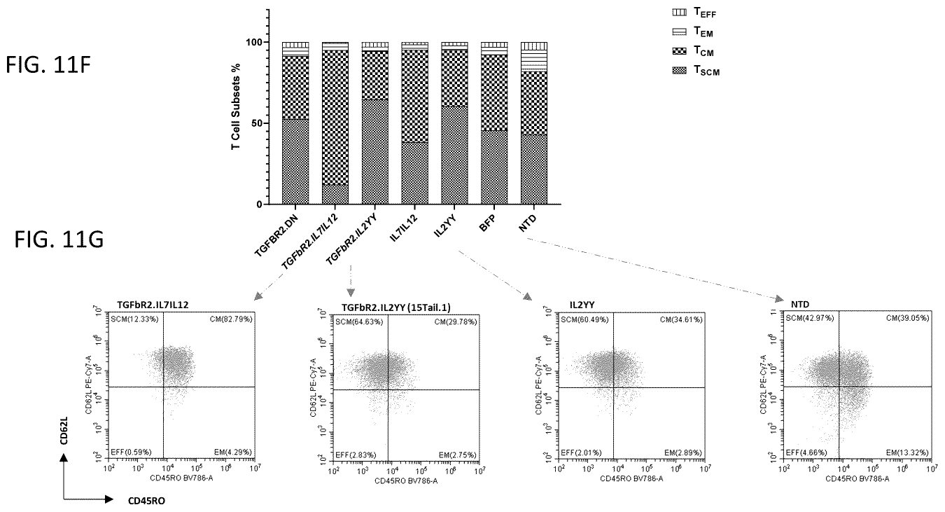

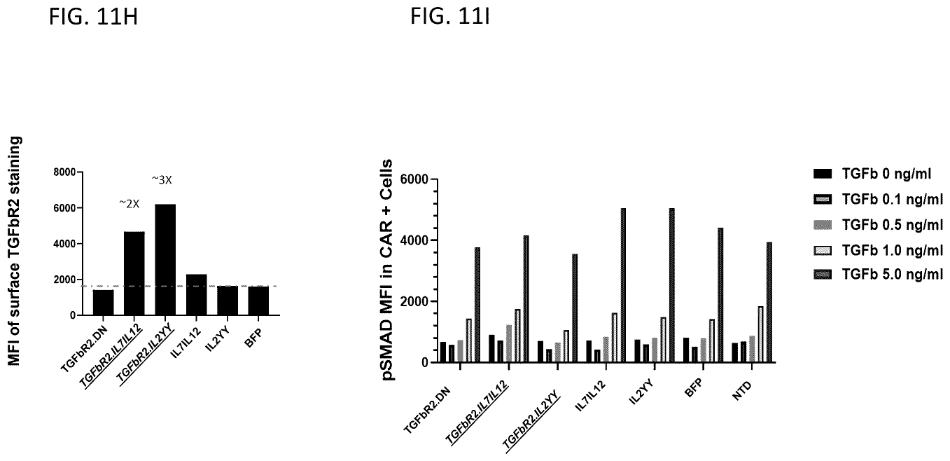

We next tested the constitutive chimeric receptors in CAR T cells. All constructs tested in FIGS. 11-16 contain the S505N and W515K substitutions in the TPOR/MPLR transmembrane domain. FIG. 11A shows schematics of the lentiviral vectors used to co-express in CAR T cells the TGF-βR2 chimeric cytokine receptors with the EGFRvIII-specific CAR (2173scFv). All chimeric cytokine receptors carry dimerization mutations in the transmembrane domain of TPOR/MPLR (S505N, W515K, see SEQ ID NO:60), and one or more cytokine receptor recruiting domains joined in tandem to mimic signaling from one or more cytokines. “IL2YY” refers to IL2Rb (393-433, 518-551), and “IL7IL12” refers to IL7R(316-459) and IL12Rb2(775-825) joined in tandem. As controls, CAR T cells were also produced, expressing a dominant negative truncation of TGF-βR2 (“TGF-βR2.DN”), constitutive chimeric cytokine receptor without the TGF-βR2 ECD (“IL7IL12”, “IL2YY”) or a BFP protein. The method to produced CAR T cells were as described in Sommer C, et al. Preclinical Evaluation of Allogeneic CAR T Cells Targeting BCMA for the Treatment of Multiple Myeloma. Mol Ther. 2019. doi:10.1016/j.ymthe.2019.04.001 and Sommer C, et al. Allogeneic FLT3 CAR T Cells with an Off-Switch Exhibit Potent Activity against AML and Can Be Depleted to Expedite Bone Marrow Recovery. Mol Ther. 2020. doi:10.1016/j.ymthe.2020.06.022. In brief, primary T cells from a healthy donor were transduced at MOI of 5, with lentiviruses expressing the CAR and each of the TGFβR2 chimeric cytokine receptors or controls. FIG. 11B shows the percentage of CAR+ T cells at day 7, 9 and 14 during CAR T cell production. CAR T cells with a constitutive chimeric cytokine receptor as indicated (“TGF-βR2.IL7IL12,” “TGF-βR2.IL2YY,” “IL7IL12,” or “IL2YY”) show higher proliferation and enrichment as compared to BFP alone over the production period. As the result, more EGFRvIII CAR+ T cells were obtained over time as compared to CAR T expressing BFP alone without a chimeric cytokine receptor (FIG. 11C).

FIGS. 11D and 11E show results of assessing STAT 5 phosphorylation in the CAR T cells co-expressing either the various chimeric cytokine receptors or controls. The antibody used for detecting phosphorylated STAT5 was from BD Biosciences (BDB612599). CAR T cells with constitutive chimeric cytokine receptor with an TGF-βR2 ECD, i.e., TGF-βR2.IL7IL12 or TGF-βR2.IL2YY exhibited higher level of STAT5 phosphorylation, even in the absence of TGFβ, as compared to IL2YY and IL7IL12, i.e., constitutive chimeric cytokine receptors without a TGF-βR2 ECD, indicating stronger cytokine signaling with a TGF-βR2 ECD domain. The data suggest that the TGF-βR2 ECD improved the intercellular recruiting domain signaling in CAR T cells.

The data in FIGS. 11F and 11G show that the chimeric cytokine receptors regulated the differentiation of CAR T cells at Day 14 of CAR T production. CD62L and CD45RO staining was conducted using the antibodies from Biolegend (#304822) and BioLegand (#304234), respectively. Notably, CAR T cells expressing the TGF-βR2.IL7IL12 chimeric cytokine receptor largely differentiated into central memory T cells (CD62L^{hi}CD45RO^{hi})—a result likely due to strong IL12Rb signaling, while CAR T cells expressing the TGFbR2.IL2YY or IL2YY chimeric cytokine receptor exhibited an enriched population of stem cell-like (stem) memory T cell (CD62L^{hi}CD45RO^{low}), a desirable T cell phenotype that has been associated with better clinical outcome.

Next, we compared the expression of TGF-βR2 chimeric cytokine receptor to the expression of endogenous TGF-βR2 by measuring total surface ECD by flow cytometry using an anti-human TGF-βR2 polyclonal antibody (R&D Systems, FAB2411A100). The results in FIG. 11H show an MFI of the ECD staining at about 1400-2300 resulting from the endogenous TGF-βR2 in the CAR T cells expressing the 1L7IL12 or IL2YY chimeric cytokine receptor and CAR T cells expressing BFP. CAR T cells expressing a TGF-βR2 chimeric cytokine receptor (TGF-βR2.IL7IL12 and TGF-βR2.IL2YY) showed a 2-3 folds excessive MFI value, indicating a 2-3 folds higher levels of the TGF-βR2 ECD staining over the levels from endogenous TGF-βR2 receptor. FIG. 11I shows the level of SMAD phosphorylation within CAR T cells expressing different chimeric cytokine receptors or controls when exposed to various concentrations of TGFβ. The antibody used for detecting phosphorylated SMAD was from BD Biosciences (BDB562586). The results show that CAR T cells expressing the TGF-βR2.IL7IL12 and TGF-βR2.IL2YY chimeric cytokine receptors exhibited decreased SMAD phosphorylation as compared to the CAR T cells expressing the IL7IL12 and IL2YY chimeric cytokine receptors without the TGF-βR2 ectodomain, respectively.

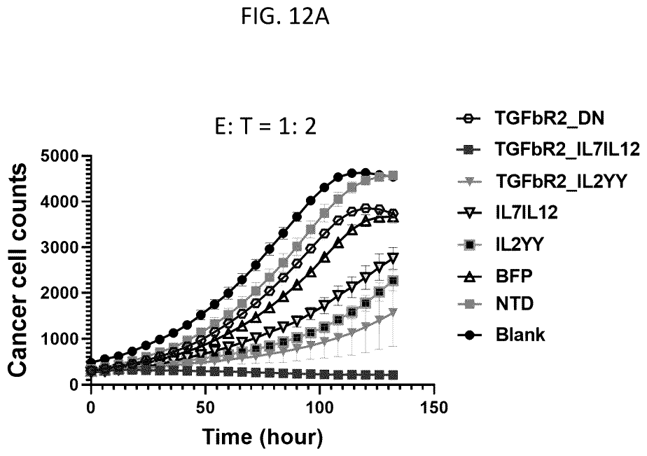

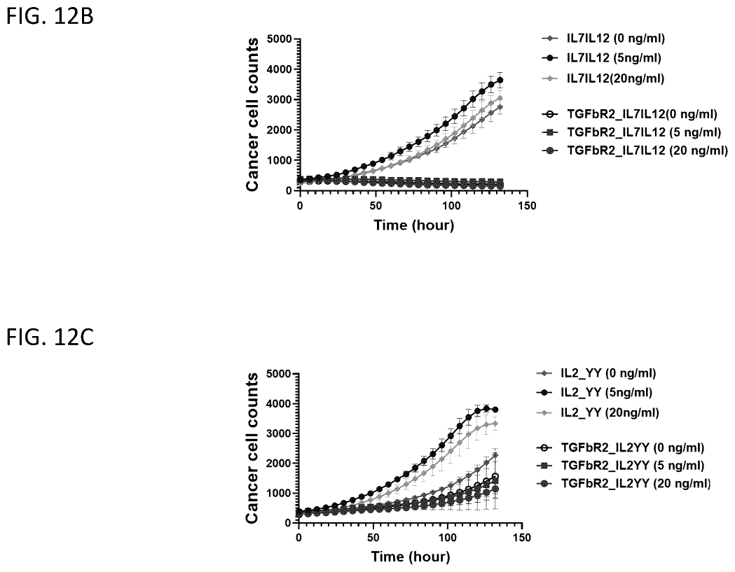

We next evaluated anti-tumor activities of the EGFRvIII CAR T cells expressing different chimeric cytokine receptors or controls against the target cells U87-EGFRvIII cells. In brief, CAR T cells were incubated with 10,000 target cells at E:T ratio of 1:2 in 200 ul of RPMI medium with 10% of FBS, and TGF-β at various concentrations of 0, 5, and 20 ng/ml. After one week of co-culture with target cells, the CAR T cells in 100 ul supernatant were transferred into new target cells (10,000) with the same TGFβ concentrations as the previous week. The cytotoxicity of the CAR T cells in the second week without added TGF-β was assessed and the results are shown in FIG. 12A. In the absence of exogenous TGFβ, CAR T cells expressing TGF-βR2 chimeric cytokine receptors (TGF-βR2.IL7IL12 and TGF-βR2.IL2YY) showed the most persistent cytotoxicity, with most of the target cells killed by the CAR T cells. CAR T cells expressing chimeric cytokine receptors without the TGF-βR2 ECD (IL7IL12 and IL2YY) also exhibited considerable cytotoxicity and inhibited the growth of the U87 cells till the second week. In comparison, CAR T cells with BFP or TGF-βR2.DN lost the activity and failed to inhibit the growth of the U87 cells (FIG. 12A). The data indicate that TGF-βR2 chimeric cytokine receptors enable more persistent cytotoxicity against the target cells. In the presence of TGFβ, the activity of CAR T cells without expressing a chimeric cytokine receptor with a TGFbR2 ECD was inhibited to various levels by TGF-β, likely the effect of signal transduction via the endogenous TGF-β receptors (FIGS. 12B-12C). In contrast, CAR T cells expressing TGF-βR2 chimeric cytokine receptors were resistant to TGF-β inhibition and were able to maintain strong cytotoxicity in conditions up to 20 ng/ml of TGF-β (FIGS. 12B, 12C).

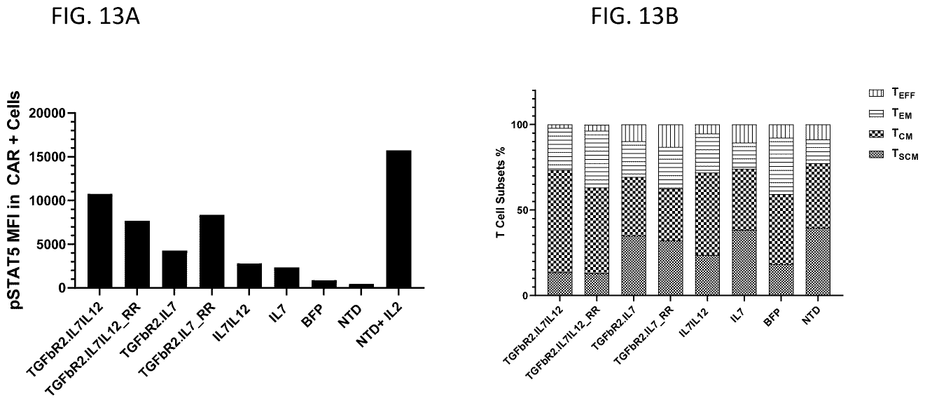

We next evaluated further modified constitutive TGF-βR2 chimeric cytokine receptor in CAR T cells. As shown above constitutive TGFβR2.IL7IL12 chimeric cytokine receptor having the S505N/W515K substitutions in the TPOR/MPLR transmembrane domain and the IL7Ra/IL12Rb recruiting domains (e.g., SEQ ID NO:163) increased STAT5 phosphorylation and led to substantial differentiation of central memory T cells. As the IL12 cytokine signaling has been implicated in the differentiation of memory T cells, we designed the TGF-βR2.IL7 chimeric cytokine receptor that eliminates the IL12 signaling. In addition, we introduced two mutations in the TpoR JAK-binding domain, K553R and K573R, (designated as “RR”, e.g., SEQ ID NOS: 165 and 170), to decrease ubiquitin-induced receptor degradation. See Saur S J, Sangkhae V, Geddis A E, Kaushansky K, Hitchcock I S. Ubiquitination and degradation of the thrombopoietin receptor c-Mpl. Blood. 2010. doi:10.1182/blood-2009-06-227033. CAR T cells expressing the chimeric cytokine receptor were produced and evaluated for STAT5 phosphorylation and T cell differentiation. Similar as before, these further modified TGF-βR2 chimeric cytokine receptors showed higher levels of STAT5 signaling as determined by STAT5 phosphorylation than the chimeric cytokine receptors without the TGF-βR2 ECD domain (both the IL7IL12 and IL7 chimeric cytokine receptor constructs contain the S505N/W515K substitutions) (FIG. 13A). In the assessment of T cell differentiation, CAR T cells expressing chimeric cytokine receptors without the IL12R recruiting domain (TGF-βR2.IL7, TGF-βR2.IL7_RR, and IL7) showed an increased proportion of stem memory T cell than their counterparts with the IL12R recruiting domain (FIG. 13B). These data demonstrate that by selecting different cytokine receptor motifs, we can modulate the TGF-βR2 chimeric cytokine receptors signaling and T cell differentiation.

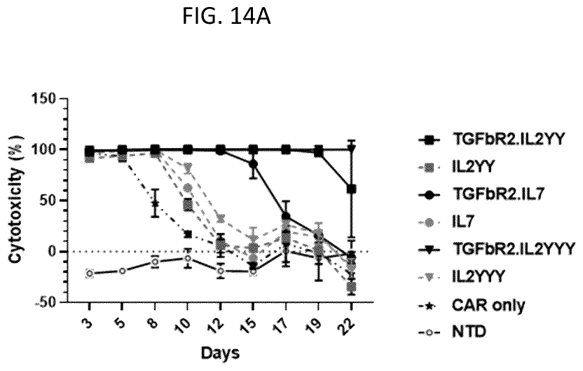

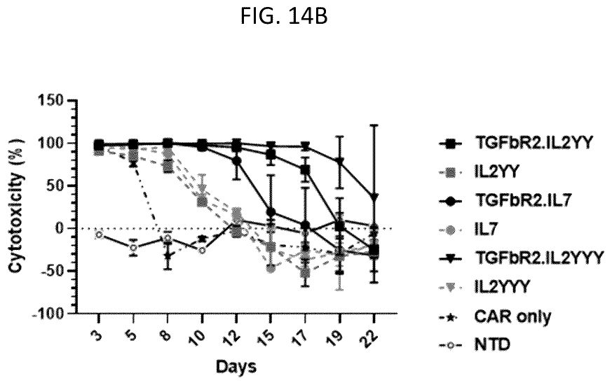

To evaluate how the TGF-βR2 chimeric cytokine receptors affect T cell functions, CAR T cells were evaluated in long-term killing assay. In brief, CAR T cells expressing different chimeric cytokine receptor were mixed with 10,000 U87-EGFRvIII cancer cells at an E:T ratio of 1:1, in 200 ul RPMI medium with 10% FBS, with or without 5 ng/ml of TGFβ. Every two or three days, 100 ul of the supernatant with CAR T cells were transferred onto 10,000 fresh target cells to the final volume of 200 ul RPMI medium with 10% FBS and 5 ng/ml of TGFβ, and the survival of old target cells were quantified. The long-term cytotoxicity of CAR T cells with different TGF-βR2 chimeric cytokine receptors are summarized in FIGS. 14A-B. In the absence of exogenous TGFβ, TGF-βR2 chimeric cytokine receptors with various recruiting domains, for example, TGF-βR2.IL2YY, TGF-βR2.1L7, or TGF-βR2.IL2YYY(IL2Rb(339-379,393-433,518-551)) all conferred more potent and durable cytotoxicity than CAR T cells expressing chimeric cytokine receptors without the TGF-βR2 ECD, confirming the previous observations that the TGF-βR2 ECD domain enhanced the chimeric cytokine receptor signaling (FIG. 14A). In the presence of 5 ng/ml TGFβ, CAR T cells expressing the TGF-βR2 chimeric cytokine receptor maintained more potent and persistent cytotoxicity than CAR T cells with or without expressing a chimeric cytokine receptor without a TGF-βR2 ECD (FIG. 14B). The chimeric cytokine receptors tested all contain the S505N/W515K substitutions, and the TGFbR2.IL2YYY and IL2YYY constructs have the additional H499L substitution in the TOPR/MPLR transmembrane domain.

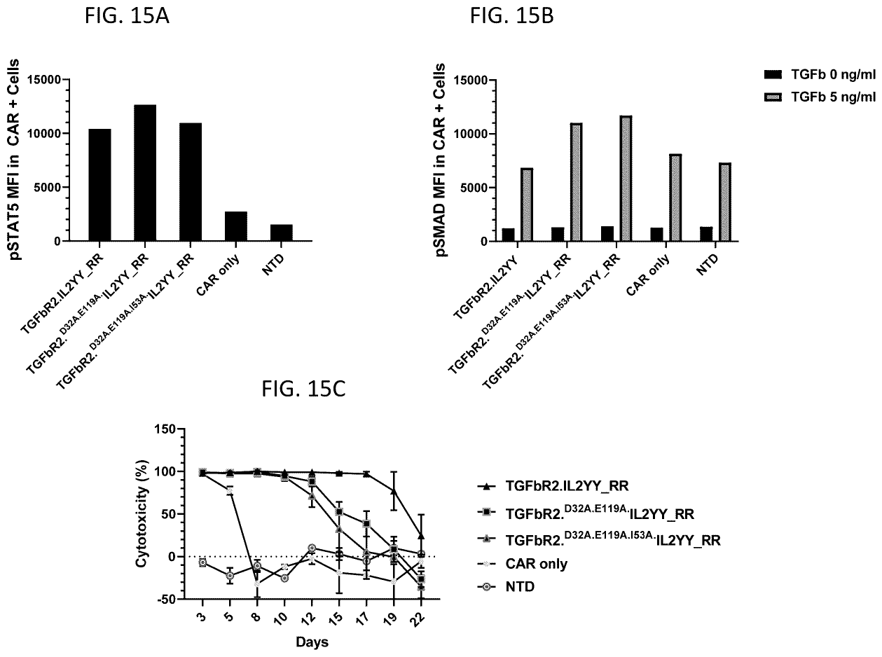

To further assess the inhibition on TGFβ signaling by a TGF-βR2 chimeric cytokine receptor and its influence on the functionality of CAR T cells, we analyzed the TGF-βR2.IL2YY_RR chimeric cytokine receptor (SEQ ID NO: 166), which carries the dimerization mutations in the TM region (S505N, W515K) and the degradation-resistant mutations (K553R and K573R) in the JAK-binding domain. We designed two additional variants bearing mutations in the TGF-βR2 ECD (D32A.E119A and D32A.E119A.I53A) that abolished the receptor's ability to bind the TGFβ ligand. CAR T cells expressing the designated chimeric cytokine receptors were produced and evaluated for cytokine signaling (pSTAT5), TGFβ signaling (pSMAD), and persistency of cytotoxicity against U87-EGFRvIII target cells. FIG. 15A shows the analysis of STAT5 signaling in the CAR T cells, in which all the TGF-βR2 chimeric cytokine receptors led to strong STAT5 phosphorylation in CAR T cells, comparing to the T cells expressing only the CAR. When the CAR T cells were exposed to 5 ng/ml of TGFβ ligand, various levels of SMAD phosphorylation were observed. Comparing to TGF-βR2 chimeric cytokine receptor with the loss-of-binding mutants, TGF-βR2.IL2YY_RR with the wildtype TGF β binding domain exhibited a decreased level of SMAD phosphorylation, indicating that the endogenous TGFβ signaling was successfully inhibited by the TGF-βR2 chimeric cytokine receptor with a wildtype, functional ligand binding domain (FIG. 15B).

The long-term cytotoxicity of CAR T cells expressing different TGF-βR2 chimeric cytokine receptor against cancer cells in the presence of 5 ng/ml TGFβ was shown in FIG. 15C. Comparing to the control CAR T cells, which quickly lost the activity to kill target cells at day 8, CAR T cells with TGF-βR2 chimeric cytokine receptor conferred more durable activity. TGF-βR2 chimeric cytokine receptors with impaired ability to bind to TGFβ (D32A.E119A and D32A.E119A.I53A) also showed a faster decline in the cytotoxicity assay than CAR T cells expressing the TGF-βR2.IL2YY_RR chimeric cytokine receptor with a wildtype TGFβ binding ECD. These data demonstrate that both the cytokine signaling and the ability to inhibit endogenous TGFβ signaling are important for the long-term activity of CAR T cells.

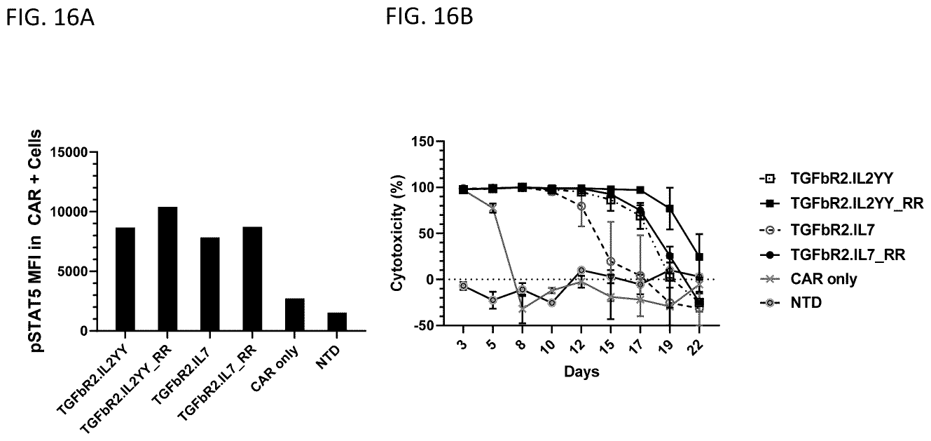

Results in FIGS. 16A-B show that the degradation-resistant mutations, K553R and K573R, can further improve the functionality of the constitutive TGF-βR2 chimeric cytokine receptors. In this experiment, CAR T cells expressing TGF-βR2 chimeric cytokine receptors with or without the degradation-resistant mutations (designated as “RR”) were evaluated for the strength of the cytokine signaling as well as the long-term cytotoxicity assay against U87-EGFRvIII in the medium with 5 ng/ml of TGFβ ligand. The TGF-βR2 chimeric cytokine receptor with the K533R and K573R substitutions consistently display a stronger STAT5 phosphorylation (FIG. 16A) and a more durable capability to kill the target cells (FIG. 16B).

1-57. (canceled)

58. A dominant-negative chimeric cytokine receptor comprising:
a) a binding domain comprising an extracellular portion of a TGF-β receptor, a TGF-β receptor variant or a TGF-β antigen binding domain; and
b) a transmembrane domain;
wherein the receptor does not comprise an intracellular domain.

59. The dominant-negative chimeric cytokine receptor of claim 58, wherein the TGF-β receptor variant comprises one or more mutations to the extracellular portion of a wild type TGF-β receptor sequence.

60. The dominant-negative chimeric cytokine receptor of claim 58, wherein the binding domain comprises the extracellular portion of TGFβR2.

61. The dominant-negative chimeric cytokine receptor of claim 58, wherein the binding domain comprises the amino acid sequence of any one of SEQ ID NOs: 2, 4-20 and 159.

62. The dominant-negative chimeric cytokine receptor of claim 58, wherein the transmembrane domain comprises a transmembrane domain of a protein selected from the group consisting of EpoR, IL6ST, PrlR, GHR, GCSFR and TPOR/MPLR.

63. The dominant-negative chimeric cytokine receptor of claim 58, wherein the receptor inhibits TGF-β signaling.

64. The dominant-negative chimeric cytokine receptor of claim 58, wherein the receptor can be induced by a TGF-β ligand or an anti-TGF βR antibody.

65. The dominant-negative chimeric cytokine receptor of claim 64, wherein the TGF-β ligand is any one of TGFβ-1, TGF-β2, or TGF-β3.

66. A polynucleotide encoding the dominant-negative chimeric cytokine receptor of claim 58.

67. A vector comprising the polynucleotide of claim 66.

68. The vector of claim 67, further comprising a polynucleotide encoding a chimeric antigen receptor (CAR).

69. The vector of claim 68, wherein the CAR is capable of specifically binding to a target selected from the group consisting of BCMA, EGFRvIII, Flt-3, WT1, CD20, CD23, CD30, CD38, CD70, CD33, CD133, MHC-WT1, TSPAN10, MHC-PRAME, Liv1, ADAM10, CHRNA2, LeY, NKG2D, CS1, CD44v6, ROR1, CD19, Claudin-18.2, Claudin-18A2, Claudin18 isoform 2, DLL3, MUC17, MUC3, Ly6G6D, c6orf23, G6D, MEGT1, NG25, E3 ubiquitin-protein ligase, RNF43, ErbB2, CEA, EpCAM, EGFR, CD40, disialoganglioside GD2 and GD3, CLL-1, ductal-epithelial mucin, gp36, TAG-72, a glycosphingolipid, glioma-associated antigen, β-human chorionic gonadotropin, AFP, lectin-reactive AFP, thyroglobulin, RAGE-1, MN-CA IX, RU1, RU2, intestinal carboxyl esterase, mutant hsp70-2, M-CSF, PSA, PAP, NY-ESO-1, LAGA-la, TP53, prostein, PSMA, survivin, PCTA-1, MAGE, ELF2M, neutrophil elastase, ephrin B2, CD22, IGFI-1, IGFI-II, IGFI receptor, mesothelin, 5T4, O1, Nkp30, tumor stromal antigen, the extra domain A (EDA) of fibronectin, the extra domain B (EDB) of fibronectin, the AI domain of tenascin-C (TnC AI), FAP, LRP6, MCSP, MARTI, MUC1, LMP2, Ras mutant, gp100, proteinase 3, bcr-abl, tyrosinase, hTERT, EphA2, ML-TAP, ERG, NA17, PAX3, ALK, Androgen receptor, CD3, CD4, CD8, CD24, CD25, CD34, CD79, CD116, CD117, CD135, CD123, CD138, CTLA-4, B7-1, B7-2, endoglin, MUC16, PSCA, Trop2, CD171, CA9, STEAP1, and VEGFR2.

70. An engineered immune cell comprising the vector of claim 69.

71. The engineered immune cell of claim 70, wherein the cell is allogeneic or autologous.

72. The engineered immune cell of claim 70, wherein the cell is selected from the group consisting of: T-cell, dendritic cell, killer dendritic cell, mast cell, NK-cell, macrophage, monocyte, B-cell and an immune cell derived from a stem cell.

73. The engineered immune cell of claim 70, wherein the immune cell exhibits reduced TGF-βR-mediated signal transduction as compared to an immune cell without expressing the dominant negative chimeric cytokine receptor.

74. The engineered immune cell of claim 73, wherein the cell exhibits reduced TGF-βR mediated signal transduction when engaged with TGF-β ligand or an anti-TGF-βR antibody.

75. A method of preparing an engineered immune cell, the method comprising introducing the polynucleotide of claim 66 into an immune cell.

76. A pharmaceutical composition comprising the engineered immune cells of claim 70.

77. A method of treating a cancer or an autoimmune disorder in a subject, the method comprising administering to the subject a therapeutically effective amount of the pharmaceutical composition of claim 76.
